(19) United States
(12) Patent Application Publication
(10) Pub. No.: US 2013/0164248 A1
(43) Pub. Date: Jun. 27, 2013

(54) COSMETIC COMPOSITION COMPRISING A PARTICULAR SILICON DERIVATIVE AND ONE OR MORE ACRYLIC THICKENING POLYMERS

(71) Applicant: Samira Khenniche, Clichy (FR)

(72) Inventor: Samira Khenniche, Clichy (FR)

(73) Assignee: L'OREAL, Paris (FR)

(21) Appl. No.: 13/821,662

(22) PCT Filed: Oct. 11, 2011
(86) PCT No.: PCT/EP2011/067683

§ 371 Date: Mar. 8, 2013

Related U.S. Application Data
(60) Provisional application No. 61/393,120, filed on Oct. 14, 2010.
(60) Provisional application No. 61/394,016, filed on Oct. 18, 2010.
(60) Provisional application No. 61/394,014, filed on Oct. 18, 2010.
(60) Provisional application No. 61/394,436, filed on Oct. 19, 2010.
(60) Provisional application No. 61/394,433, filed on Oct. 19, 2010.
(60) Provisional application No. 61/394,438, filed on Oct. 19, 2010.
(60) Provisional application No. 61/394,431, filed on Oct. 19, 2010.
(60) Provisional application No. 61/394,823, filed on Oct. 20, 2010.
(60) Provisional application No. 61/394,820, filed on Oct. 20, 2010.

(30) Foreign Application Priority Data
Oct. 12, 2010 (FR) 1058268
Oct. 12, 2010 (FR) 1058269
Oct. 12, 2010 (FR) 1058270
Oct. 12, 2010 (FR) 1058272
Oct. 12, 2010 (FR) 1058273
Oct. 12, 2010 (FR) 1058274
Oct. 12, 2010 (FR) 1058275
Oct. 12, 2010 (FR) 1058276
Oct. 12, 2010 (FR) 1058293

(51) Int. Cl. A61K 8/898; A61Q 5/12

(52) U.S. Cl. CPC A61K 8/898; A61Q 5/12
USPC 424/70.122

(57) ABSTRACT

The invention relates to a cosmetic composition comprising: (i) one or more products (A) of the reaction of: (a) one or more oxirane or oxetane compounds comprising at least two oxirane or oxetane groups; and (b) one or more aminosilanes corresponding to the formula (1a): N(H)(R¹)R²Si(OR³)_{3-a-b-c}(OR⁴)ₐ(R⁵Si(OR⁶)_{d}(R⁷)ₑ)_{b}R⁸_{c} (1a) in which: R¹ is selected from the group constituted by H or a monovalent hydrocarbon-based radical comprising from 1 to 20 carbon atoms; R² is selected from the group constituted by a divalent, linear or branched, hydrocarbon-based radical, comprising from 1 to 60 carbons; R⁴ is a hydrocarbon-based radical containing 3 to 200 carbon atoms; R⁵ is selected from the group constituted by oxygen or a divalent, linear or branched, hydrocarbon-based radical comprising from 1 to 60 carbons; R³, R⁶, R⁷ and R⁸ are each independently selected from the group constituted by monovalent, linear or branched, hydrocarbon-based radicals having from 1 to 200 carbon atoms; the sub-script b is equal to zero or is a positive number and has a value ranging from 0 to 3; the subscript a is equal to zero or is a positive number below 3, the subscripts b and c are equal to zero or are positive and have a value ranging from 0 to 3 on condition that (a+b+c)<3; the subscripts d and e are equal to zero or are positive and have a value ranging from 0 to 3 on condition that (d+e)=3; and (ii) one or more acrylic thickening polymers. The invention also relates to a cosmetic method for treating keratin materials with this composition, and also to the use of this composition for caring for keratin materials.

One subject of the present invention is a cosmetic composition comprising at least one particular silicon compound and at least one particular thickening polymer.

Keratin fibres may suffer attack of diverse origins, for instance mechanical attack (disentangling or brushing), or chemical attack (dyeing or permanent wave). This attack has an impact on the qualities of the fibre and will lead to difficult disentangling at the time of washing the hair, and to non-smooth, dry and uneven surface properties when the hair is dry. The hair is difficult to style and lacks softness.

The conditioning compositions that are currently proposed comprise essentially cationic surfactants, fatty substances, silicones and cationic polymers. They make it possible to facilitate disentangling by softening the keratin fibre and provide sheen, softness, and uniformity to dried hair. However, these effects do not withstand shampooing, and the application of these compositions is essential at each washing step in order to treat and facilitate the disentangling of the hair.

The results obtained are not completely satisfactory both as regards the cosmetic properties themselves and their durability.

There thus exists a need to have available more effective conditioning compositions.

Firstly, the Applicant Company has discovered, surprisingly, that compositions comprising at least one particular thickening polymer and at least one particular silicon compound make it possible to solve the problems touched on above.

More specifically, one subject of the invention is a cosmetic composition comprising:

(i) one or more products (A) of the reaction of:
(a) one or more oxirane or oxetane compounds comprising at least two oxirane or oxetane groups; and
(b) one or more aminosilanes corresponding to the formula (Ia):

N(H)(R¹)R²Si(OR³)_{3-a-b-c}(OR⁴)ₐ(R⁵Si(OR⁶)_{d}(R⁷)ₑ)_{b}R^{a}_{c} (Ia)

in which:
R¹ is selected from the group constituted by H or a monovalent hydrocarbon-based radical comprising from 1 to 20 carbon atoms;
R² is selected from the group constituted by a divalent, linear or branched, hydrocarbon-based radical, comprising from 1 to 60 carbons;
R⁴ is a hydrocarbon-based radical containing 3 to 200 carbon atoms;
R⁵ is selected from the group constituted by an oxygen atom or a divalent, linear or branched, hydrocarbon-based radical, comprising from 1 to 60 carbons;
R³, R⁶, R⁷ and R⁸ are each independently selected from the group constituted by monovalent, linear or branched, hydrocarbon-based radicals having from 1 to 200 carbon atoms;
the subscript b is equal to zero or is a positive number and has a value ranging from 0 to 3;
the subscript a is equal to zero or is a positive number below 3, the subscripts b and c are equal to zero or are positive and have a value ranging from 0 to 3 on condition that (a+b+c)≦3;
the subscripts d and e are equal to zero or are positive and have a value ranging from 0 to 3 on condition that (d+e)=3; and
(ii) one or more acrylic thickening polymers.

Secondly, the Applicant Company has discovered, surprisingly, that compositions comprising at least one particular surfactant and at least one particular silicon compound make it possible to solve the problems touched on above.

More specifically, one subject of the invention is a cosmetic composition comprising:

(i) one or more products (A) of the reaction:
a) of one or more oxirane or oxetane compounds comprising at least two oxirane or oxetane groups; and
b) of one or more aminosilanes corresponding to the formula (Ia):

N(H)(R¹)R²Si(OR³)_{3-a-b-c}(OR⁴)ₐ(R⁵Si(OR⁶)_{d}(R⁷)ₑ)_{b}R⁸_{c} (Ia)

where R¹ is selected from the group constituted by H or a monovalent hydrocarbon-based radical comprising from 1 to 20 carbon atoms;
R² is selected from the group constituted by a divalent, linear or branched, hydrocarbon-based radical, comprising from 1 to 60 carbons;
R⁴ is a hydrocarbon-based radical containing 3 to 200 carbon atoms;
R⁵ is selected from the group constituted by oxygen or a divalent, linear or branched, hydrocarbon-based radical comprising from 1 to 60 carbons;
R³, R⁶, R⁷ and R⁸ are each independently selected from the group constituted by monovalent, linear or branched, hydrocarbon-based radicals having from 1 to 200 carbon atoms;
the subscript b is equal to zero or is a positive number and has a value ranging from 0 to 3;
the subscript a is equal to zero or is a positive number below 3, the subscripts b and c are equal to zero or are positive and have a value ranging from 0 to 3 on condition that (a+b+c)≦3;
the subscripts d and e are equal to zero or are positive and have a value ranging from 0 to 3 on condition that (d+e)=3,
(ii) one or more amphoteric surfactants of betaine type.

Thirdly, the Applicant Company has discovered, surprisingly, that compositions comprising at least one particular silicone and at least one particular silicon compound make it possible to solve the problems touched on above.

More specifically, one subject of the invention is a cosmetic composition comprising:

(i) one or more products (A) of the reaction:
a) of one or more oxirane or oxetane compounds comprising at least two oxirane or oxetane groups; and
b) of one or more aminosilanes corresponding to the formula:

N(H)(R¹)R²Si(OR³)_{3-a-b-c}(OR⁴)ₐ(R⁵Si(OR⁶)_{d}(R⁷)ₑ)_{b}R⁸_{c}

where R¹ is selected from the group constituted by H or a monovalent hydrocarbon-based radical comprising from 1 to 20 carbon atoms;
R² is selected from the group constituted by a divalent, linear or branched, hydrocarbon-based radical, comprising from 1 to 60 carbons;
R⁴ is a hydrocarbon-based radical containing 3 to 200 carbon atoms;
R⁵ is selected from the group constituted by oxygen or a divalent, linear or branched, hydrocarbon-based radical comprising from 1 to 60 carbons;
R³, R⁶, R⁷ and R⁸ are each independently selected from the group constituted by monovalent, linear or branched, hydrocarbon-based radicals having from 1 to 200 carbon atoms;
the subscript b is equal to zero or is a positive number and has a value ranging from 0 to 3;
the subscript a is equal to zero or is a positive number below 3, the subscripts b and c are equal to zero or are positive and have a value ranging from 0 to 3 on condition that (a+b+c)≦3;
the subscripts d and e are equal to zero or are positive and have a value ranging from 0 to 3 on condition that (d+e)=3,
(ii) one or more organomodified silicones different from the compounds (i).

Fourthly, the Applicant Company has discovered, surprisingly, that compositions comprising at least one cationic polymer and at least one particular silicon compound make it possible to solve the problems touched on above.

More specifically, one subject of the invention is a cosmetic composition comprising:

(i) one or more products (A) of the reaction:
a) of one or more oxirane or oxetane compounds comprising at least two oxirane or oxetane groups; and
b) of one or more aminosilanes corresponding to the formula:

N(H)(R¹)R²Si(OR³)_{3-a-b-c}(OR⁴)ₐ(R⁵Si(OR⁸)_{d}(R⁷)ₑ)_{b}R⁸_{c}

where R¹ is selected from the group constituted by H or a monovalent hydrocarbon-based radical comprising from 1 to 20 carbon atoms;
R² is selected from the group constituted by a divalent, linear or branched, hydrocarbon-based radical, comprising from 1 to 60 carbons;
R⁴ is a hydrocarbon-based radical containing 3 to 200 carbon atoms;
R⁵ is selected from the group constituted by oxygen or a divalent, linear or branched, hydrocarbon-based radical comprising from 1 to 60 carbons;
R³, R⁶, R⁷ and R⁸ are each independently selected from the group constituted by monovalent, linear or branched, hydrocarbon-based radicals having from 1 to 200 carbon atoms;
the subscript b is equal to zero or is a positive number and has a value ranging from 0 to 3;
the subscript a is equal to zero or is a positive number below 3, the subscripts b and c are equal to zero or are positive and have a value ranging from 0 to 3 on condition that (a+b+c)≦3;
the subscripts d and e are equal to zero or are positive and have a value ranging from 0 to 3 on condition that (d+e)=3,
(ii) one or more cationic polymers.

Fifthly, the Applicant Company has discovered, surprisingly, that compositions comprising at least one particular surfactant and at least one particular silicon compound make it possible to solve the problems touched on above.

More specifically, one subject of the invention is a cosmetic composition comprising:

(i) one or more products (A) of the reaction:
a) of one or more oxirane or oxetane compounds comprising at least two oxirane or oxetane groups; and
b) of one or more aminosilanes corresponding to the formula:

N(H)(R¹)R²Si(OR³)_{3-a-b-c}(OR⁴)ₐ(R⁵Si(OR⁶)_{d}(R⁷)ₑ)_{b}R⁸_{c}

where R¹ is selected from the group constituted by H or a monovalent hydrocarbon-based radical comprising from 1 to 20 carbon atoms;
R² is selected from the group constituted by a divalent, linear or branched, hydrocarbon-based radical, comprising from 1 to 60 carbons;
R⁴ is a hydrocarbon-based radical containing 3 to 200 carbon atoms;
R⁵ is selected from the group constituted by oxygen or a divalent, linear or branched, hydrocarbon-based radical comprising from 1 to 60 carbons;
R³, R⁶, R⁷ and R⁸ are each independently selected from the group constituted by monovalent, linear or branched, hydrocarbon-based radicals having from 1 to 200 carbon atoms;
the subscript b is equal to zero or is a positive number and has a value ranging from 0 to 3;
the subscript a is equal to zero or is a positive number below 3, the subscripts b and c are equal to zero or are positive and have a value ranging from 0 to 3 on condition that (a+b+c)≦3;
the subscripts d and e are equal to zero or are positive and have a value ranging from 0 to 3 on condition that (d+e)=3,
(ii) one or more cationic surfactants comprising one or more permanent positive charges.

Sixthly, the Applicant Company has discovered, surprisingly, that compositions comprising at least one particular surfactant and at least one particular silicon compound make it possible to solve the problems touched on above.

More specifically, one subject of the invention is a cosmetic composition comprising:

(i) one or more products (A) of the reaction:
a) of one or more oxirane or oxetane compounds comprising at least two oxirane or oxetane groups; and
b) of one or more aminosilanes corresponding to the formula:

N(H)(R¹)R²Si(OR³)_{3-a-b-c}(OR⁴)ₐ(R⁵Si(OR⁶)_{d}(R⁷)ₑ)_{b}R⁸_{c}

where R¹ is selected from the group constituted by H or a monovalent hydrocarbon-based radical comprising from 1 to 20 carbon atoms;
R² is selected from the group constituted by a divalent, linear or branched, hydrocarbon-based radical, comprising from 1 to 60 carbons;
R⁴ is a hydrocarbon-based radical containing 3 to 200 carbon atoms;
R⁵ is selected from the group constituted by oxygen or a divalent, linear or branched, hydrocarbon-based radical comprising from 1 to 60 carbons;
R³, R⁶, R⁷ and R⁸ are each independently selected from the group constituted by monovalent, linear or branched, hydrocarbon-based radicals having from 1 to 200 carbon atoms;
the subscript b is equal to zero or is a positive number and has a value ranging from 0 to 3;
the subscript a is equal to zero or is a positive number below 3, the subscripts b and c are equal to zero or are positive and have a value ranging from 0 to 3 on condition that (a+b+c)≦3;
the subscripts d and e are equal to zero or are positive and have a value ranging from 0 to 3 on condition that (d+e)=3,
(ii) one or more anionic surfactants comprising one or more sulphate or sulphonate groups.

Furthermore, in view of combatting dandruff in the hair or on the scalp, which may generally be accompanied by microbial and/or fungal proliferation, among the antidandruff products that have been proposed are either products that inhibit microbial proliferation or keratolytic products.

However, hair treated with antidandruff agents has a coarse, laden feel. In addition, this hair is relatively difficult to disentangle. Furthermore, the effects are not durable, in particular with respect to shampooing operations.

The objective of the present invention is therefore to propose compositions for effectively combating dandruff while at the same time providing good cosmetic properties, in particular in terms of softness and disentangling of the hair.

The use of conditioning agents, in particular cationic or amphoteric polymers or silicones, to make the hair easier to disentangle and to give it softness and manageability has already been recommended in compositions for washing or caring for keratin materials such as the hair. However, the cosmetic advantages mentioned above are unfortunately also accompanied, on dried hair, by certain cosmetic effects that are considered as being undesirable, namely lankness of the hairstyle (lack of lightness of the hair) and a lack of smoothness (hair not uniform from the root to the tip).

In addition, the use of cationic polymers for this purpose presents various drawbacks. On account of their high affinity for the hair, some of these polymers become deposited in a large amount during repeated use, and give undesirable effects such as an unpleasant, laden feel, stiffening of the hair and an inter-fibre adhesion which affects the styling. These drawbacks are accentuated in the case of fine hair, which lacks liveliness and body.

In summary, it is found that the current cosmetic compositions containing antidandruff agents are not entirely satisfactory.

Thus, following considerable research conducted in this matter, the Applicant Company has now found that by introducing a specific silicon derivative into the compositions, in particular hair compositions based on antidandruff agents, it is possible to increase the deposition of the antidandruff agent, its efficacy and the durability of the effect, while at the same time improving the cosmetic properties of the compositions based on antidandruff agents.

More specifically, one subject of the invention is a cosmetic or dermatological composition comprising:

(i) one or more products (A) of the reaction:
a) of one or more oxirane or oxetane compounds comprising at least two oxirane or oxetane groups; and
b) of one or more aminosilanes corresponding to the formula:

N(H)(R¹)R²Si(OR³)_{3-a-b-c}(OR⁴)ₐ(R⁵Si(OR⁶)_{d}(R⁷)ₑ)_{b}R⁸_{c}

where R¹ is selected from the group constituted by H or a monovalent hydrocarbon-based radical comprising from 1 to 20 carbon atoms;
R² is selected from the group constituted by a divalent, linear or branched, hydrocarbon-based radical, comprising from 1 to 60 carbons;
R⁴ is a hydrocarbon-based radical containing 3 to 200 carbon atoms;
R⁵ is selected from the group constituted by oxygen or a divalent, linear or branched, hydrocarbon-based radical comprising from 1 to 60 carbons;
R³, R⁶, R⁷ and R⁸ are each independently selected from the group constituted by monovalent, linear or branched, hydrocarbon-based radicals having from 1 to 200 carbon atoms;
the subscript b is equal to zero or is a positive number and has a value ranging from 0 to 3;
the subscript a is equal to zero or is a positive number below 3, the subscripts b and c are equal to zero or are positive and have a value ranging from 0 to 3 on condition that (a+b+c)≦3;
the subscripts d and e are equal to zero or are positive and have a value ranging from 0 to 3 on condition that (d+e)=3,
(ii) one or more antidandruff agents selected from:
(1) pyridinethione salts,
(2) 1-hydroxy-2-pyrrolidone derivatives,
(3) 2,2′-dithiobis(pyridine-N-oxide),
(4) trihalocarbamides,
(5) triclosan,
(6) azole compounds such as climbazole, ketoconazole, clotrimazole, econazole, isoconazole and miconazole,
(7) selenium sulphides,
(8) extracts of one or more non-photosynthetic, non-fruiting filamentous bacteria.

Furthermore, the cosmetic compositions for hair shaping and/or form retention of the hairstyle that are the most widely available on the cosmetics market are spray compositions consisting essentially of a solution, usually an alcoholic solution, and of one or more components, known as fixing components, which are generally polymer resins, the role of which is to form welds between the hairs. These fixing components are often formulated as a mixture with various cosmetic adjuvants. These cosmetic compositions are generally packaged either in a pump-dispenser bottle, or in a suitable aerosol container pressurized using a propellant, the aerosol system then contains, on the one hand, a liquid phase (or fluid) and, on the other hand, a propellant.

Once applied to the hair, the liquid phase, containing the fixing components and a suitable solvent, dries enabling the formation of welds necessary for the fixing of the hair by the fixing components. The welds have to be sufficiently rigid to provide the form retention of the hair; however, they must also be sufficiently weak for the user to be able, by combing or by brushing the hair, to destroy them without hurting the scalp or damaging the hair.

The conventional film-forming resins generally used as fixing agent in an alcoholic medium exhibit the disadvantage of conferring mediocre cosmetic properties on the styling composition; in particular, the feel obtained by the use of the compositions based on film-forming resins is not very satisfactory.

To improve the feel of these compositions, it has often been the practice in the prior art to add silicones, however this solution does not make it possible to completely satisfactorily improve the cosmetic properties in the case of compositions containing a polar solvent.

The Applicant Company has found, surprisingly and advantageously, that the use of a combination of a particular silicon compound and of a fixing polymer makes it possible to improve the cosmetic properties of these compositions.

The compositions according to the present invention allow good fixing and good form retention of the hair, i.e. a styling effect that lasts throughout the day, or even for several days, which exhibits good resistance to moisture and is easy to remove by shampooing.

These compositions also make it possible to give the hair good cosmetic properties, especially in terms of feel (soft feel) and disentangling.

It has also been observed that the combination according to the invention allows perfect suspension of solid particles such as flakes, and that the thixotropic nature of this combination allows perfect restitution of the styling cosmetic composition when it is applied using a spray or as an aerosol.

One subject of the invention is a cosmetic composition comprising:
(i) one or more products (A) of the reaction:
a) of one or more oxirane or oxetane compounds comprising at least two oxirane or oxetane groups; and
b) of one or more aminosilanes corresponding to the formula:

N(H)(R¹)R²Si(OR³)_{3-a-b-c}(OR⁴)ₐ(R⁵Si(OR⁶)_{d}(R⁷)ₑ)_{b}R⁸_{c}

where R¹ is selected from the group constituted by H or a monovalent hydrocarbon-based radical comprising from 1 to 20 carbon atoms;
R² is selected from the group constituted by a divalent, linear or branched, hydrocarbon-based radical, comprising from 1 to 60 carbons;
R⁴ is a hydrocarbon-based radical containing 3 to 200 carbon atoms;
R⁵ is selected from the group constituted by oxygen or a divalent, linear or branched, hydrocarbon-based radical comprising from 1 to 60 carbons;
R³, R⁶, R⁷ and R⁸ are each independently selected from the group constituted by monovalent, linear or branched, hydrocarbon-based radicals having from 1 to 200 carbon atoms;
the subscript b is equal to zero or is a positive number and has a value ranging from 0 to 3;
the subscript a is equal to zero or is a positive number below 3, the subscripts b and c are equal to zero or are positive and have a value ranging from 0 to 3 on condition that (a+b+c)≦3;
the subscripts d and e are equal to zero or are positive and have a value ranging from 0 to 3 on condition that (d+e)=3,
(ii) one or more fixing polymers selected from anionic, cationic, amphoteric or nonionic fixing polymers, or mixtures thereof;
(iii) one or more solvents having a dielectric constant greater than 15 and less than 100, at 20° C.

Finally, the present invention also relates to a reducing cosmetic composition, for the first time for an operation for permanently reshaping the hair, comprising at least one reducing agent and at least one particular silicon compound. It also relates to a process for permanently reshaping the hair using said composition.

The technique most commonly used to permanently reshape the hair consists, in a first step, in opening the disulphide bonds of the keratin (cystine) using a composition containing a reducing agent, and then, preferably after having rinsed the hair, in reforming said disulphide bonds, in a second step, by applying to the hair, which has been smoothed or placed under tension beforehand using suitable means such as curlers, an oxidizing composition also known as a “fixative”, so as to give the hair the desired shape. This technique makes it possible either to wave the hair or to straighten, crimp or smooth it.

These common techniques for transforming the shape of the hair, in particular compositions for permanent-waving or smoothing the hair, based on thiol-containing products, lead to satisfactory shaping results, but the use of these products also leads to degradations of the fibre and an impairment of their cosmetic properties. Furthermore, both the shaping properties and the cosmetic properties degrade over the course of successive washing operations.

There is therefore a need for novel cosmetic compositions intended for an operation for permanently reshaping the hair which do not have the drawbacks of the prior art, in particular which degrade the hair and its cosmetic properties less and which give lasting results.

which do not induce any disturbance in the colour uptake during successive hair permanent-waving and hair colouring operations.

Above all, the following are desired:
on wet hair: smoothness, uniformity, a slippery coating that facilitates combing;
on dry hair: smoothness, softness, uniformity, ease of combing, sheen. These properties must withstand several shampooing operations.

The Applicant Company has discovered, surprisingly, that the use, in a reducing composition for permanently reshaping keratin fibres, of one particular silicon compound provides an improvement over the prior art.

One subject of the invention is a reducing cosmetic composition for permanently reshaping keratin fibres, in particular the hair, characterized in that it comprises:

(i) one or more reducing agents,
(ii) one or more products (A) of the reaction:
a) of one or more oxirane or oxetane compounds comprising at least two oxirane or oxetane groups; and
b) of one or more aminosilanes corresponding to the formula:

N(H)(R¹)R²Si(OR³)_{3-a-b-c}(OR⁴)ₐ(R⁵Si(OR⁶)_{d}(R⁷)ₑ)_{b}R⁸_{c}

where R¹ is selected from the group constituted by H or a monovalent hydrocarbon-based radical comprising from 1 to 20 carbon atoms;
R² is selected from the group constituted by a divalent, linear or branched, hydrocarbon-based radical, comprising from 1 to 60 carbons;
R⁴ is a hydrocarbon-based radical containing 3 to 200 carbon atoms;
R⁵ is selected from the group constituted by oxygen or a divalent, linear or branched, hydrocarbon-based radical comprising from 1 to 60 carbons;
R³, R⁶, R⁷ and R⁸ are each independently selected from the group constituted by monovalent, linear or branched, hydrocarbon-based radicals having from 1 to 200 carbon atoms;
the subscript b is equal to zero or is a positive number and has a value ranging from 0 to 3;
the subscript a is equal to zero or is a positive number below 3, the subscripts b and c are equal to zero or are positive and have a value ranging from 0 to 3 on condition that (a+b+c)≦3;
the subscripts d and e are equal to zero or are positive and have a value ranging from 0 to 3 on condition that (d+e)=3.

Such compositions are easily distributed over the hair and are easily removed with water resulting in clean, shiny, smooth and soft hair which is easily disentangled and the good cosmetic properties of which endure over time, even after several shampooing operations.

A further subject of the invention is a method for the nontherapeutic cosmetic treatment of keratin materials, in particular the hair, comprising the application, to said materials, of a composition as described above.

Another subject of the invention is the use of a composition according to the invention for caring for keratin materials, especially keratin fibres and in particular human keratin fibres such as the hair.

The composition according to the invention comprises one or more products (A) of the reaction of:

(a) one or more oxirane or oxetane compounds comprising at least two oxirane or oxetane groups; and
(b) one or more aminosilanes corresponding to the formula (Ia):

N(H)(R¹)R²Si(OR³)_{3-a-b-c}(OR⁴)ₐ(R⁵Si(OR⁶)_{d}(R⁷)ₑ)_{b}R⁸_{c} (Ia)

in which:
R¹ is selected from the group constituted by H or a monovalent hydrocarbon-based radical comprising from 1 to 20 carbon atoms;
R² is selected from the group constituted by a divalent, linear or branched, hydrocarbon-based radical, comprising from 1 to 60 carbons;
R⁴ is a hydrocarbon-based radical containing 3 to 200 carbon atoms;
R⁵ is selected from the group constituted by an oxygen atom or a divalent, linear or branched, hydrocarbon-based radical, comprising from 1 to 60 carbons;
R³, R⁶, R⁷ and R⁸ are each independently selected from the group constituted by monovalent, linear or branched, hydrocarbon-based radicals having from 1 to 200 carbon atoms;
the subscript b is equal to zero or is a positive number and has a value ranging from 0 to 3;
the subscript a is equal to zero or is a positive number below 3, the subscripts b and c are equal to zero or are positive and have a value ranging from 0 to 3 on condition that (a+b+c)≦3;
the subscripts d and e are equal to zero or are positive and have a value ranging from 0 to 3 on condition that (d+e)=3.

In one variant of the invention, the oxirane or oxetane compound(s) is (are) selected from the group constituted by siloxanes, hydrocarbons and polyethers.

More preferably still, the oxirane or oxetane compound is:
either a siloxane corresponding to formula (Ib):

MᵣM^{E}ₕM^{PE}ᵢM^{H}ⱼDₖD^{E}ₗD^{PE}ₘD^{H}ₙTₒT^{E}ₚT^{PE}_{q}T^{H}ᵣQₛ with

### M=R⁹R¹⁰R¹¹SiO_{1/2};

### M^{H}=R¹²R¹³HSiO_{1/2};

M^{PE}=R¹²R¹³(—CH₂CH(R¹⁴)(R¹⁵)ₜO(R¹⁶)ᵤ(C₂H₄O)ᵥ(C₃H₆O)_{w}(C₄H₈O)ₓR¹⁷)SiO_{1/2};

### M^{E}=R¹²R¹³(R^{E})SiO_{1/2};

### D=R¹⁸R¹⁹SiO_{2/2}; and

### D^{H}=R²⁰HSiO_{2/2};

D^{PE}=R²⁰(—CH₂CH(R¹⁴)(R¹⁵)ₜO(R¹⁶)ᵤ(C₂H₄O)ᵥ(C₃H₆O)_{w}(C₄H₈O)ₓR¹⁷)SiO_{2/2};

### D^{E}=R²⁰HSiO_{2/2};

### T=R²¹SiO_{3/2};

### T^{H}=HSiO_{3/2};

T^{PE}=(—CH₂CH(R¹⁴)(R¹⁵)ₜO(R¹⁶)ᵤ(C₂H₄O)ᵥ(C₃H₆O)_{w}(C₄H₈O)ₓR¹⁷)SiO_{3/2};

### T^{E}=R^{E}SiO_{3/2}; and

### Q=SiO_{4/2};

where R⁹, R¹⁰, R¹¹, R¹², R¹³, R¹⁸, R¹⁹, R²⁰ and R²¹ are each independently selected from the group constituted by monovalent, hydrocarbon-based radicals having from 1 to 60 carbon atoms;
R¹⁴ is H or an alkyl group having 1 to 6 carbon atoms; R¹⁵ is a divalent alkyl radical having 1 to 6 carbons; R¹⁶ is selected from the group constituted by divalent radicals constituted by —C₂H₄O—, —C₃H₆O—, and —C₄H₈O—; R¹⁷ is H, a monofunctional hydrocarbon-based radical having 1 to 6 carbons, or acetyl;
R^{E} is independently a monovalent hydrocarbon-based radical containing one or more oxirane or oxetane units having from one to sixty carbon atoms; the subscript f may be equal to zero or be positive on condition that when the subscript f is equal to zero, h must be positive;
the subscript h may be equal to zero or be positive on condition that when h is equal to zero, the subscript f must be positive; and that the sum of the subscripts h, l and p is positive;
the subscript k is equal to zero or is positive and has a value ranging from around 0 to around 1000;
the subscript l is equal to zero or is positive and has a value ranging from around 0 to around 400 on condition that the sum of the subscripts h, l and p is positive;
the subscript o is equal to zero or is positive and has a value ranging from 0 to around 50;
the subscript p is equal to zero or is positive and has a value ranging from 0 to around 30 on condition that the sum of the subscripts h, l and p is positive;
the subscript s is equal to zero or is positive and has a value ranging from 0 to around 20;
the subscript i is equal to zero or is positive and has a value ranging from 0 to around 20;
the subscript m is equal to zero or is positive and has a value ranging from 0 to around 200;
the subscript q is equal to zero or is positive and has a value ranging from 0 to around 30;
the subscript j is equal to zero or is positive and has a value ranging from 0 to around 2;
the subscript n is equal to zero or is positive and has a value ranging from 0 to around 20;
the subscript r is equal to zero or is positive and has a value ranging from 0 to around 30;
the subscript t is equal to zero or one;
the subscript u is equal to zero or one;
the subscript v is equal to zero or is positive and has a value ranging from 0 to around 100 on condition that (v+w+x)>0;
the subscript w is equal to zero or is positive and has a value ranging from 0 to around 100 on condition that (v+w+x)>0;
the subscript x is equal to zero or is positive and has a value ranging from 0 to around 100 on condition that (v+w+x)>0;
or a hydrocarbon corresponding to formula (Ic):

R²²_{y}(R²³)_{z}(R²⁴_{α})(R²⁵)_{β} (Ic)

where R²² and R²⁵ are independently a monovalent hydrocarbon-based radical containing one or more oxirane or oxetane units having from 3 to 12 carbon atoms;
R²³ and R²⁴ are each selected from the group constituted by H or a monovalent, linear or branched, hydrocarbon-based radical having 1 to 200 carbons;
the subscripts y, z, α and β are equal to zero or are positive ranging from zero to four, on condition that (y+β)>2;
or a polyether corresponding to formula (Id):

R²⁶O(R²⁷)_{γ}(C₂H₄O)_{δ}(C₃H₆O)_{∈}(C₄H₈O)_{ξ}²⁸ (Id)

where R²⁶ and R²⁸ are independently a monovalent hydrocarbon-based radical containing one or more oxirane or oxetane units having from 3 to 12 carbon atoms;
R²⁷ is selected from the group constituted by divalent radicals constituted by —C₂H₄O—, —C₃H₆O—, and —C₄H₈O—;
the subscript γ is equal to zero or 1;
the subscript δ is equal to zero or is positive and has a value ranging from 0 to around 100 on condition that (δ+∈+ξ)>0;
the subscript E is equal to zero or is positive and has a value ranging from 0 to around 100 on condition that (δ+∈+ξ)>0;
the subscript ξ is equal to zero or is positive and has a value ranging from 0 to around 100 on condition that (δ+∈+ξ)>0.

The synthesis of the product (A) may, besides an oxirane or oxetane compound and an aminosilane, also use a compound corresponding to formula (Ie):

R²⁹(R³⁰)_{κ}Si(OR³¹)_{3-η-θ}(R³²)_{η}(OR³³)_{θ} (Ie)

where R²⁹ is a monovalent hydrocarbon-based radical containing one or more oxirane or oxetane units having from 3 to 12 carbon atoms;
R³⁰ is a divalent hydrocarbon-based radical constituted of 1 to 60 carbons and the subscript κ has a value of zero or 1; R³¹ and R³² are independently selected from the group constituted by monovalent, linear or branched, hydrocarbon-based radicals having from 1 to 60 carbon atoms;
the subscript η is equal to zero or is positive and has a value ranging from 0 to 3;
the subscript θ is greater than 0 and less than or equal to 3, on condition that 3-η-θ is greater than or equal to zero;
R³³ is a hydrocarbon-based radical containing from 3 to 200 carbon atoms.

As used in this text, the expression “hydrocarbon-based radical” is understood to mean hydrocarbon-based radicals that may optionally be substituted with heteroatoms, in particular nitrogen, oxygen and sulphur, and that may optionally contain cyclic structures such as oxirane and oxetane groups.

During the reaction of oxirane or oxetane compounds with amino compounds, the molar ratio of the oxirane or epoxy groups to the amino groups preferably ranges from around 1 to around 4, more preferably it is greater than around 1.1 and less than around 3.9, and very preferably greater than around 1.2 and less than around 3.8. R¹ is preferably a monovalent hydrocarbon-based radical having 1 to around 10 carbon atoms or hydrogen, more preferably having 1 to around 5 carbon atoms or hydrogen, very preferably R¹ is H. R² is preferably a monovalent hydrocarbon-based radical having 1 to around 10 carbon atoms, more preferably having 2 to around 8 carbon atoms, and very preferably having 3 to around 5 carbon atoms. R⁴ is preferably a monovalent hydrocarbon-based radical having 3 to around 10 carbon atoms, more preferably having 3 to around 8 carbon atoms, very preferably having 3 to around 5 carbon atoms. R³, R⁶, R⁷, and R⁸ are each preferably a monovalent hydrocarbon-based radical having 1 to around 20 carbon atoms, more preferably having 1 to around 15 carbon atoms, very preferably having 2 to around 8 carbon atoms. The subscript a is in the range extending from 0 to around 3, preferably from around 1 to around 3, more preferably from around 2 to around 3, very preferably from 0 to around 1. The subscript b is in the range extending from 0 to around 25, more preferably from 0 to around 15 and very preferably 3. The subscript c is in the range extending from 0 to around 3, more preferably from 0 to around 2, very preferably from 0 to around 1. R⁹, R¹⁰, R¹¹, R¹², R¹³, R¹⁸, R¹⁹, R²⁰ and R²¹ are each preferably a monovalent hydrocarbon-based radical having 1 to around 4 carbon atoms, more preferably having 1 to around 3 carbon atoms, and very preferably having 1 carbon atom. The subscripts f, l, m, n, s are each in the range extending from 0 to around 200, more preferably from 0 to around 100, and very preferably from 0 to around 50. The subscript k is in the range extending from 0 to around 500, more preferably from 5 to around 250, and very preferably from 5 to around 150. The subscripts v, w and x are each in the range extending from 0 to around 50, more preferably from 0 to around 35, and very preferably from 0 to around 25. R²³ and R²⁴ are each preferably a monovalent hydrocarbon-based radical ranging from 5 to around 1000 carbon atoms, more preferably from 10 to around 500, and very preferably from 10 to around 300. The subscripts δ, ∈, ξ are in the range extending from 0 to around 50, more preferably from 0 to around 30, and very preferably from 0 to around 15. R³¹ and R³² are each preferably a monovalent hydrocarbon-based radical having 1 to around 10 carbon atoms, more preferably having 1 to around 8 carbon atoms, and very preferably having 1 to around 4 carbon atoms. R³³ are each preferably a monovalent hydrocarbon-based radical having 3 to around 100 carbon atoms, more preferably having 3 to around 50 carbon atoms, very preferably having 3 to around 10 carbon atoms.

Additional materials c) may be added to the initial mixture of synthetic precursors. These additional materials may be reactive or non-reactive. The distinctive feature of the present invention is that the reaction product is obtained from the reaction of at least the components listed as disclosed. Non-reactive components may be added to the reaction mixture as diluents or to impart additional properties unrelated to the properties of the composition prepared as reaction product. Thus, for example, finely divided solid materials such as pigments may be dispersed in the reaction mixture, before, during or after the reaction, in order to produce a reaction product composition that comprises, in addition, the non-reactive component, for example a pigment. Additional reactive components may also be added; such components may react with the initial reactants or they may react with the reaction product; the expression “reaction product” is intended to include these possibilities, and also to include the addition of non-reactive components.

Optionally, the reaction of component a) with component b) and optionally with compound c) may be carried out in the presence of a primary or secondary amine which may or may not possess a reactive alkoxysilane unit. The result will be a reaction product of a), b), and of the primary or secondary amine. Examples of these primary amines are: methylamine, ethylamine, propylamine, ethanolamine, isopropylamine, butylamine, isobutylamine, hexylamine, dodecylamine, oleylamine, aniline, aminopropyltrimethylsilane, aminopropyltriethylsilane, aminomorpholine, aminopropyldiethylamine, benzylamine, naphthylamine, 3-amino-9-ethylcarbazole, 1-aminoheptaphlorohexane, 2,2,3,3,4,4,5,5,6,6,7,7,8,8,8-pentadecafluoro-1-octanamine, and the like. Examples of secondary amines are: methylethylamine, methylhexylamine, methyloctadecylamine, diethanolamine, dibenzylamine, dihexylamine, dicyclohexylamine, piperidine, pyrrolidine, phthalimide, and the like. Polymeric amines may also be used.

The product (A) of the reaction of a), an oxirane or oxetane compound having two or more oxirane or oxetane groups per molecule, and of b), an aminosilane, results in a polymer that contains alkoxysilane functional units covalently bound to the polymer chain.

The products (A) may be used in the compositions of the invention in solubilized form or in the form of a dispersion.

One of the variants of the invention consists in using the products (A) in the form of emulsions or microemulsions.

The synthesis of the compounds (A) is especially described in patent application WO 2009/061360.

The reaction product(s) (A) may be present in the cosmetic composition according to the invention in a content ranging from 0.01% to 20%, in particular from 0.05 to 15%, preferably from 0.5 to 15%, relative to the total weight of the cosmetic composition.

The composition according to the invention may therefore comprise one or more acrylic thickening polymers.

The expression “acrylic polymer” is understood, for the purposes of the present invention, to mean a polymer that results from the polymerization of one or more monomers, including at least one or more monomers of the following structure (II):

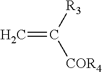

R₃ denoting a hydrogen atom or a linear or branched C₁-C₄ alkyl radical;
R₄ denoting a hydrogen atom, a linear or branched C₁-C₄ alkyl radical, an NR₅R₆ radical, or a linear or branched C₁-C₃₀ alkoxy radical, optionally substituted with one or more hydroxyl radicals or with a quaternary ammonium radical;
R₅ and R₆ denote a hydrogen atom or an optionally oxyalkylenated C₁-C₃₀ alkyl radical, the alkyl radical possibly comprising a sulphonic group.

Preferably, R₃ denotes a hydrogen atom or a methyl radical.

The expression “thickening polymer” is understood, for the purposes of the present invention, to mean a polymer having, in solution or in dispersion containing 1% by weight of active material in water or in ethanol at 25° C., a viscosity greater than 0.2 poise at a shear rate of 1 s⁻¹. The viscosity can be measured with a HAAKE RS600 viscometer from THERMO ELECTRON. This viscometer is a controlled-stress viscometer with cone-plate geometry (for example having a diameter of 60 mm).

The thickening polymers are especially selected from:

(a) acrylic associative thickeners;
(b) crosslinked acrylic acid homopolymers;
(c) crosslinked copolymers of (meth)acrylic acid and of (C₁-C₆)alkyl acrylate;
(d) nonionic homopolymers and copolymers containing ethylenically unsaturated monomers of ester and/or amide type;
(e) ammonium acrylate homopolymers or copolymers of ammonium acrylate and of acrylamide;
(f) (meth)acrylamido(C₁-C₄)alkylsulphonic acid homopolymers and copolymers;
(g) crosslinked methacryloyl(C₁-C₄)alkyltri(C₁-C₄)alkylammonium homopolymers and copolymers.

The expression “associative thickener” is understood according to the invention to mean an amphiphilic thickener comprising both hydrophilic units and hydrophobic units, in particular comprising at least one C₈-C₃₀ fatty chain and at least one hydrophilic unit.

(a) Acrylic associative thickeners that may be used according to the invention are acrylic associative polymers selected from:
(i) nonionic amphiphilic polymers comprising at least one fatty chain and at least one hydrophilic unit;
(ii) anionic amphiphilic polymers comprising at least one hydrophilic unit and at least one fatty-chain unit;
(iii) cationic amphiphilic polymers comprising at least one hydrophilic unit and at least one fatty-chain unit;
(iv) amphoteric amphiphilic polymers comprising at least one hydrophilic unit and at least one fatty-chain unit; the fatty chains containing from 10 to 30 carbon atoms.
(i) The acrylic nonionic amphiphilic polymers comprising at least one fatty chain and at least one hydrophilic unit are preferably selected from:
(1) copolymers of C₁-C₆ alkyl methacrylates or acrylates and of amphiphilic monomers comprising at least one fatty chain (for example oxyethylenated (C₈-C₂₂)alkyl acrylates), for instance the oxyethylenated methyl methacrylate/stearyl acrylate copolymer sold by the company Goldschmidt under the name Antil 208;
(2) copolymers of hydrophilic methacrylates or acrylates and of hydrophobic monomers comprising at least one fatty chain (for example (C₈-C₂₂)alkyl (meth)acrylates), for instance polyethylene glycol methacrylate/lauryl methacrylate copolymer.
(ii) The acrylic anionic amphiphilic polymers can be selected from those comprising at least one hydrophilic unit of unsaturated olefinic carboxylic acid type, and at least one hydrophobic unit of (C₁₀-C₃₀)alkyl ester of an unsaturated carboxylic acid type. They are preferably selected from those in which the hydrophilic unit of unsaturated olefinic carboxylic acid type corresponds to the monomer of formula (III) below:

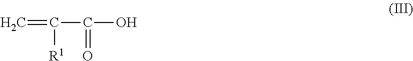

in which formula R¹ denotes H or CH₃ or C₂H₅, i.e. acrylic acid, methacrylic acid or ethacrylic acid units, and the hydrophobic unit of which, of (C₁₀-C₃₀)alkyl ester of an unsaturated carboxylic acid type, corresponds to the monomer of formula (IV) below:

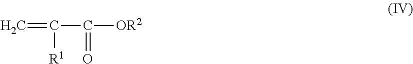

in which formula R¹ denotes H or CH₃ or C₂H₅ (i.e. acrylate, methacrylate or ethacrylate units) and preferably H (acrylate units) or CH₃ (methacrylate units), R² denoting a C₁₀-C₃₀ and preferably C₁₂-C₂₂ alkyl radical.
(C₁₀-C₃₀) Alkyl esters of unsaturated carboxylic acids according to the invention include, for example, lauryl acrylate, stearyl acrylate, decyl acrylate, isodecyl acrylate and dodecyl acrylate, and the corresponding methacrylates, lauryl methacrylate, stearyl methacrylate, decyl methacrylate, isodecyl methacrylate and dodecyl methacrylate.

Anionic amphiphilic polymers of this type are disclosed and prepared, for example, according to the U.S. Pat. No. 3,915,921 and U.S. Pat. No. 4,509,949.

The anionic amphiphilic polymers that can be used in the context of the present invention may more particularly denote polymers formed from a mixture of monomers comprising:

(i) acrylic acid and one or more esters of formula (V) below:

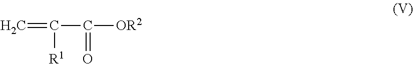

in which R¹ denotes H or CH₃, R² denoting an alkyl radical having from 12 to 22 carbon atoms, and a crosslinking agent, such as, for example, those constituted of from 95% to 60% by weight of acrylic acid (hydrophilic unit), 4% to 40% by weight of C₁₀-C₃₀ alkyl acrylate (hydrophobic unit), and 0 to 6% by weight of crosslinking polymerizable monomer, or 98% to 96% by weight of acrylic acid (hydrophilic unit), 1% to 4% by weight of C₁₀-C₃₀ alkyl acrylate (hydrophobic unit) and 0.1% to 0.6% by weight of crosslinking polymerizable monomer,
(ii) essentially acrylic acid and lauryl methacrylate, such as the product formed from 66% by weight of acrylic acid and 34% by weight of lauryl methacrylate.

Said crosslinking agent is a monomer containing a

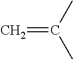

group with at least one other polymerizable group whose unsaturated bonds are not conjugated relative to one another. Mention may be made in particular of polyallyl ethers such as, in particular, polyallyl sucrose and polyallyl pentaerythritol.

Among said polymers above, the ones most particularly preferred according to the present invention are the products sold by the company Goodrich under the trade names Pemulen TR1, Pemulen TR2, Carbopol 1382, and more preferably still Pemulen TR1, and the product sold by the company S.E.P.C. under the name Coatex SX.

As anionic amphiphilic fatty-chain polymers, mention may also be made of the copolymer of methacrylic acid, methyl acrylate and dimethyl-meta-isopropenylbenzyl isocyanate of ethoxylated alcohol sold under the name Viscophobe DB 1000 by the company Amerchol.

Other fatty-chain anionic amphiphilic polymers that may be mentioned include those comprising at least one acrylic monomer containing sulphonic group(s), in free or partially or totally neutralized form and comprising at least one hydrophobic portion.

The hydrophobic portion present in the polymers of the invention preferably contains from 8 to 22 carbon atoms, more preferably still from 8 to 18 carbon atoms and more particularly from 12 to 18 carbon atoms.

Preferably, the sulphonic polymers in accordance with the invention are partially or totally neutralized with an inorganic base (sodium hydroxide, potassium hydroxide or aqueous ammonia) or an organic base such as mono-, di- or triethanolamine, an aminomethylpropanediol, N-methylglucamine, basic amino acids, for instance arginine and lysine, and mixtures of these compounds.

The sulphonic amphiphilic polymers in accordance with the invention generally have a number-average molecular weight ranging from 1000 to 20 000 000 g/mol, preferably ranging from 20 000 to 5 000 000 and more preferably still from 100 000 to 1 500 000 g/mol.

The sulphonic amphiphilic polymers according to the invention may or may not be crosslinked. Crosslinked amphiphilic polymers are preferably selected.

When they are crosslinked, the crosslinking agents may be selected from polyolefinically unsaturated compounds commonly used for the crosslinking of polymers obtained by free-radical polymerization. Mention may be made, for example, of divinylbenzene, diallyl ether, dipropylene glycol diallyl ether, polyglycol diallyl ethers, triethylene glycol divinyl ether, hydroquinone diallyl ether, ethylene glycol di(meth)acrylate or tetraethylene glycol di(meth)acrylate, trimethylolpropane triacrylate, methylenebisacrylamide, methylenebismethacrylamide, triallylamine, triallyl cyanurate, diallyl maleate, tetraallylethylenediamine, tetraallyloxyethane, trimethylolpropane diallyl ether, allyl (meth)acrylate, allyl ethers of alcohols of the sugar series, or other allyl or vinyl ethers of polyfunctional alcohols, and also allyl esters of phosphoric and/or vinylphosphonic acid derivatives, or mixtures of these compounds.

Methylenebisacrylamide, allyl methacrylate or trimethylolpropane triacrylate (TMPTA) will be used more particularly. The degree of crosslinking will generally range from 0.01 mol % to 10 mol % and more particularly from 0.2 mol % to 2 mol % relative to the polymer.

The acrylic monomers containing sulphonic group(s) are selected especially from (meth)acrylamido(C₁-C₂₂)alkylsulphonic acids and N—(C₁-C₂₂)alkyl(meth)acrylamido(C₁-C₂₂)alkylsulphonic acids, for instance undecylacrylamidomethanesulphonic acid, and also partially or totally neutralized forms thereof.

Use will more preferably be made of (meth)acrylamido(C₁-C₂₂)alkylsulphonic acids, such as, for example, acrylamidomethanesulphonic acid, acrylamidoethanesulphonic acid, acrylamidopropanesulphonic acid, 2-acrylamido-2-methylpropanesulphonic acid, methacrylamido-2-methylpropanesulphonic acid, 2-acrylamido-n-butanesulphonic acid, 2-acrylamido-2,4,4-trimethylpentanesulphonic acid, 2-methacrylamidododecylsulphonic acid, 2-acrylamido-2,6-dimethyl-3-heptanesulphonic acid, and also partially or totally neutralized forms thereof.

2-Acrylamido-2-methylpropanesulphonic acid (AMPS), and also partially or totally neutralized forms thereof, will more particularly be used.

The amphiphilic polymers in accordance with the invention may be selected especially from random amphiphilic AMPS polymers modified by reaction with a C₆-C₂₂ n-monoalkylamine or di-n-alkylamine, and such as those described in Patent Application WO 00/31154. These polymers may also contain other ethylenically unsaturated hydrophilic monomers selected, for example, from (meth)acrylic acids, β-substituted alkyl derivatives thereof or esters thereof obtained with monoalcohols or mono- or polyalkylene glycols, (meth)acrylamides, vinylpyrrolidone, maleic anhydride, itaconic acid or maleic acid, or mixtures of these compounds.

The preferred polymers of the invention are selected from amphiphilic copolymers of AMPS and of at least one ethylenically unsaturated hydrophobic monomer comprising at least one hydrophobic portion containing from 8 to 50 carbon atoms, more preferably from 8 to 22 carbon atoms, more preferably still from 8 to 18 carbon atoms and more particularly 12 to 18 carbon atoms.

These same copolymers may also contain one or more ethylenically unsaturated monomers not comprising a fatty chain, such as (meth)acrylic acids, β-substituted alkyl derivatives thereof or esters thereof obtained with monoalcohols or mono- or polyalkylene glycols, (meth)acrylamides, vinylpyrrolidone, maleic anhydride, itaconic acid or maleic acid, or mixtures of these compounds.

These copolymers are described especially in Patent Application EP-A-750 899, U.S. Pat. No. 5,089,578 and in the following publications by Yotaro Morishima:
Self-assembling amphiphilic polyelectrolytes and their nanostructures, Chinese Journal of Polymer Science, Vol. 18, No. 40, (2000), 323-336;
Micelle formation of random copolymers of sodium 2-(acrylamido)-2-methylpropanesulphonate and a nonionic surfactant macromonomer in water as studied by fluorescence and dynamic light scattering, Macromolecules, 2000, Vol. 33, No. 10-3694-3704;
Solution properties of micelle networks formed by nonionic moieties covalently bound to a polyelectrolyte: salt effects on rheological behavior—Langmuir, 2000, Vol. 16, No. 12, 5324-5332;
Stimuli responsive amphiphilic copolymers of sodium 2-(acrylamido)-2-methylpropanesulphonate and associative macromonomers, Polym. Preprint, Div. Polym. Chem. 1999, 40(2), 220-221.

The ethylenically unsaturated hydrophobic monomers of these particular copolymers are preferably selected from the acrylates or acrylamides of formula (VI) below:

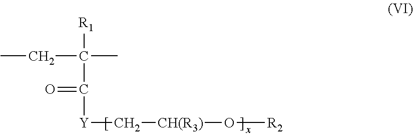

in which R₁ and R₃, which may be identical or different, denote a hydrogen atom or a linear or branched C₁-C₆ alkyl radical (preferably methyl); Y denotes O or NH; R₂ denotes a hydrophobic hydrocarbon-based radical containing at least from 8 to 50 carbon atoms, more preferably from 8 to 22 carbon atoms, more preferably still from 6 to 18 carbon atoms and more particularly from 12 to 18 carbon atoms; x denotes a number of moles of alkylene oxide and ranges from 0 to 100.

The R₂ radical is preferably selected from linear C₆-C₁₈ alkyl radicals (for example n-hexyl, n-octyl, n-decyl, n-hexadecyl or n-dodecyl radicals); branched C₆-C₁₈ alkyl radicals; cyclic C₆-C₁₈ alkyl radicals (for example cyclododecane (C₁₂) or adamantane (C₁₀) radicals); C₆-C₁₈ alkylperfluorinated radicals (for example the group of formula —(CH₂)₂—(CF₂)₉—CF₃); the cholesteryl (C₂₇) radical or a cholesterol ester residue, such as the cholesteryl oxyhexanoate group; or polycyclic aromatic groups, such as naphthalene or pyrene. Among these radicals, the ones that are more particularly preferred are linear alkyl radicals and more particularly the n-dodecyl radical.

According to one particularly preferred form of the invention, the monomer of formula (VI) comprises at least one alkylene oxide unit (x≧1) and preferably a polyoxyalkylenated chain. The polyoxyalkylenated chain is preferably constituted of ethylene oxide units and/or of propylene oxide units and more particularly still constituted of ethylene oxide units. The number of oxyalkylene units generally ranges from 3 to 100, more preferably from 3 to 50 and more preferably still from 7 to 25.

Among these polymers, mention may be made of:
copolymers, which may or may not be crosslinked and which may or may not be neutralized, comprising from 15 to 60% by weight of AMPS units and from 40 to 85% by weight of (C₈-C₁₆)alkyl(meth)acrylamide units or of (C₈-C₁₆)alkyl (meth)acrylate units, with respect to the polymer, such as those described in Application EP-A-750 899;
terpolymers comprising from 10 mol % to 90 mol % of acrylamide units, from 0.1 mol % to 10 mol % of AMPS units and from 5 mol % to 80 mol % of n-(C₆-C₁₈)alkylacrylamide units, such as those described in U.S. Pat. No. 5,089,578.

Mention may also be made of copolymers of totally neutralized AMPS and of dodecyl methacrylate, and also crosslinked and non-crosslinked copolymers of AMPS and of n-dodecylmethacrylamide, such as those described in the Morishima articles mentioned above.

Mention will be made more particularly of the copolymers constituted of 2-acrylamido-2-methylpropanesulphonic acid (AMPS) units of formula (VII) below:

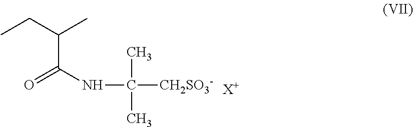

in which X⁺is a proton, an alkali metal cation, an alkaline-earth metal cation or the ammonium ion,
and units of formula (VIII) below:

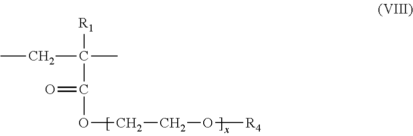

in which x denotes an integer varying from 3 to 100, preferably from 5 to 80, and more preferably from 7 to 25; R₁ has the same meaning as that indicated above in formula (I) and R₄ denotes a linear or branched C₆-C₂₂ and more preferably C₁₀-C₂₂ alkyl.

The polymers that are particularly preferred are those for which x=25, R₁ denotes methyl and R₄ represents n-dodecyl; they are described in the Morishima articles mentioned above.

The polymers for which X⁺denotes sodium or ammonium are more particularly preferred.

(iii) The cationic amphiphilic polymers used in the present invention are preferably selected from polyacrylates containing amine side groups.

The polyacrylates containing quaternized or non-quaternized amine side groups contain, for example, hydrophobic groups of the type of steareth-20 (polyoxyethylenated (20) stearyl alcohol) or (C₁₀-C₃₀)alkyl PEG-20 itaconate.

Examples of polyacrylates containing amino side chains that may be mentioned are the polymers 8781-124B or 9492-103 or Structure Plus from the company National Starch.

(iv) As amphoteric amphiphilic polymers containing at least one fatty chain, mention may be made of methacrylamidopropyltrimethylammonium chloride/acrylic acid/C₁₀-C₃₀ alkyl methacrylate copolymers, the alkyl radical preferably being a stearyl radical.
(b) Mention may be made, among crosslinked acrylic acid homopolymers, of those crosslinked by an allyl ether of an alcohol from the sugar series, such as, for example, the products sold under the names Carbopol 980, 981, 954, 2984 and 5984 by the company Goodrich or the products sold under the names Synthalen M and Synthalen K by the company 3 VSA.
c) Mention may be made, among crosslinked copolymers of (meth)acrylic acid and of C₁-C₆ alkyl acrylate, of the product sold under the name Viscoatex 538C by the company Coatex, which is a crosslinked copolymer of methacrylic acid and of ethyl acrylate as an aqueous dispersion containing 38% active material, or the product sold under the name Aculyn 33 by the company Rohm & Haas, which is a crosslinked copolymer of acrylic acid and of ethyl acrylate as an aqueous dispersion containing 28% active material. Mention may be made more particularly of the crosslinked methacrylic acid/ethyl acrylate copolymer in the form of an aqueous 30% dispersion manufactured and sold under the name Carbopol Aqua SF-1 by the company Noveon.
(d) Mention may be made, among nonionic homopolymers or copolymers comprising ethylenically unsaturated monomers of ester and/or amide type, of the products sold under the names of: Cyanamer P250 by the company Cytec (polyacrylamide); PMMA MBX-8C by the company US Cosmetics (methyl methacrylate/ethylene glycol dimethacrylate copolymer); Acryloid B66 by the company Rohm & Haas (butyl methacrylate/methyl methacrylate copolymer); BPA 500 by the company Kobo (polymethyl methacrylate).
(e) Mention may be made, among ammonium acrylate homopolymers, of the product sold under the name Microsap PAS 5193 by the company Hoechst. Mention may be made, among the copolymers of ammonium acrylate and of acrylamide, of the product sold under the name Bozepol C Nouveau or the product PAS 5193 sold by the company Hoechst (they are described and prepared in documents FR-2 416 723, U.S. Pat. No. 2,798,053 and U.S. Pat. No. 2,923,692).
(f) Poly(meth)acrylamido(C₁-C₄)alkylsulphonic acids.

According to the present invention, the poly(meth)acrylamido(C₁-C₄)alkylsulphonic acid(s) is (are) preferably crosslinked.

Even more particularly, they are partially or totally neutralized.

These are water-soluble or water-swellable polymers.

Among these polymers, mention may especially be made of:
polyacrylamidomethanesulphonic acid,
polyacrylamidoethanesulphonic acid,
polyacrylamidopropanesulphonic acid,
poly-2-acrylamido-2-methylpropanesulphonic acid,
poly-2-methacrylamido-2-methylpropanesulphonic acid,
poly-2-acrylamido-n-butanesulphonic acid.

Polymers of this type and especially crosslinked and partially or totally neutralized poly(2-acrylamido-2-methylpropanesulphonic acid)s are known, described and prepared in document DE-196 25 810.

They are generally characterized by the fact that they comprise, randomly distributed:

a) from 90% to 99.9% by weight of units of formula (IX) below:

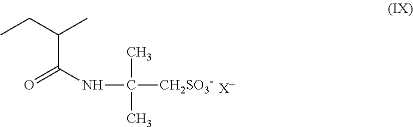

in which X⁺denotes a cation or a mixture of cations, including H⁺,
b) from 0.01% to 10% by weight of at least one crosslinking unit containing at least two olefinic double bonds,
the weight proportions being defined relative to the total weight of the polymer;
X⁺represents a cation or a mixture of cations selected in particular from a proton, an alkali metal cation, a cation equivalent to that of an alkaline-earth metal, or an ammonium ion.

The crosslinked and neutralized poly(2-acrylamido-2-methylpropanesulphonic acid) preferably comprises from 98% to 99.5% by weight of units of formula (IX) and from 0.5% to 2% by weight of crosslinking units.

The crosslinking units containing at least two olefinic double bonds are selected, for example, from dipropylene glycol diallyl ether, polyglycol diallyl ethers, triethylene glycol divinyl ether, hydroquinone diallyl ether, tetraallyloxyethane or other polyfunctional alcohol allyl or vinyl ethers, tetraethylene glycol diacrylate, triallylamine, trimethylolpropane diallyl ether, methylenebisacrylamide or divinylbenzene.

The crosslinking units containing at least two olefinic double bonds are even more particularly selected from those corresponding to the general formula (X) below:

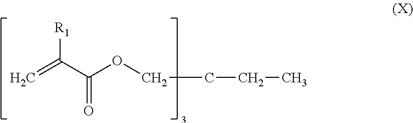

in which R₁ denotes a hydrogen atom or a C₁-C₄ alkyl and more particularly methyl (trimethylolpropane triacrylate).

The crosslinked and partially or totally neutralized poly(2-acrylamido-2-methyl-propanesulphonic acids) are generally known under the names “Ammonium polyacrylamido-2-methylpropanesulphonate” or “Ammonium polyacryldimethyl-tauramide” (INCI name).

A product that is particularly preferred according to the invention is the one sold by the company Clariant under the trade name Hostacerin AMPS; this is a crosslinked poly(2-acrylamido-2-methylpropanesulphonic acid) partially neutralized with aqueous ammonia.

(g) Crosslinked polymers of methacryloyloxy(C₁-C₄)alkyltri(C₁-C₄)alkylammonium salts, such as the polymers obtained by homopolymerization of dimethylaminoethyl methacrylate quaternized with methyl chloride, or by copolymerization of acrylamide with dimethylaminoethyl methacrylate quaternized with methyl chloride, the homopolymerization or copolymerization being followed by crosslinking with an olefinically unsaturated compound, more particularly methylenebisacrylamide. A crosslinked acrylamide/methacryloyloxyethyltrimethyl-ammonium chloride copolymer (20/80 by weight) in the form of a dispersion containing 50% by weight of said copolymer in mineral oil can be used more particularly. This dispersion is sold under the name Salcare® SC 92 by the company Ciba. Use may also be made of a crosslinked homopolymer of methacryloyloxyethyltrimethylammonium chloride containing approximately 50% by weight of the homopolymer in mineral oil or in a liquid ester. These dispersions are sold under the names Salcare® SC 95 and Salcare® SC 96 by the company Ciba.

According to the invention, the acrylic thickening polymer(s) may represent from 0.001% to 20% by weight, preferably from 0.01% to 10% by weight and more particularly from 0.1% to 5% by weight relative to the total weight of the composition.

As indicated previously, the cosmetic composition according to the invention may comprise one or more amphoteric surfactants of betaine type. The amphoteric surfactant(s) of betaine type may especially be of formula (I):

R₁—(CONH)ₓ-A₁-N⁺(R₂)(R₃)-A₂-Z (I)

with
x denoting 0 or 1,
A₁ and A₂ denoting, independently of one another, a linear or branched C₁-C₁₀ alkylene radical optionally substituted with a hydroxyl radical,
R₁ denoting a linear or branched C₆-C₃₀ alkyl or alkenyl radical,
R₂ and R₃ denoting, independently of one another, a linear or branched C₁-C₄ alkyl radical,
Z denoting a CO₂⁻ group or an SO₃⁻group.

Preferably, R₂ and R₃ denote a methyl radical.

The amphoteric surfactant(s) of betaine type used in the cosmetic composition according to the present invention may especially be (C₈₋₂₀)alkylbetaines, (C₈-C₂₀)alkylsulphobetaines, (C₈₋₂₀ alkyl)amido(C₂₋₈ alkyl)betaines or (C₈₋₂₀ alkyl)amido(C₆₋₈ alkyl)sulphobetaines.

Among the amphoteric surfactants mentioned above that are preferably used are (C₈₋₂₀ alkyl)betaines and (C₈₋₂₀ alkyl)amido(C₂₋₈ alkyl)betaines, and mixtures thereof.

More particularly, the amphoteric surfactants of betaine type ii) are selected from cocobetaine and cocamidopropylbetaine.

The amphoteric surfactant(s) of betaine type ii) may be present in a content ranging from 0.01% to 20% by weight, preferably in a content ranging from 0.1% to 15% by weight and more preferably still in a content ranging from 0.5% to 10% by weight, and better still 1% to 8% by weight relative to the total weight of the cosmetic composition according to the invention.

The product(s) (i)/amphoteric surfactant of betaine type ii) weight ratio ranges from 0.01 to 100, more preferably still from 0.1 to 50, better still from 1 to 10.

The composition according to the invention may comprise organomodified silicones.

The silicones are polyorganosiloxanes (or polysiloxanes) that may be in the form of aqueous solutions, i.e. dissolved, or optionally in the form of dispersions or microdispersions, or of aqueous emulsions. The polyorganosiloxanes may also be in the form of oils, waxes, resins or gums.

Organopolysiloxanes are defined in greater detail in Walter Noll's “Chemistry and Technology of Silicones”, (1968), Academic Press.

The organomodified silicones present in the composition according to the invention are silicones as defined above and comprising in their structure one or more organofunctional groups attached via a hydrocarbon-based radical.

Among the organomodified silicones, mention may be made of polyorganosiloxanes comprising:
polyethyleneoxy and/or polypropyleneoxy groups optionally comprising C₆-C₂₄ alkyl groups, such as the oxyethylenated and oxypropylenated poly(methyllauryl/methylsiloxane) sold under the name Dow Corning 5200 Formulation Aid by the company Dow Corning (INCI: Lauryl PEG/PPG-18/18 methicone), the products known as dimethicone copolyol sold by the company Dow Corning under the name DC 1248 or the oils Silwet L 722, L 7500, L 77 and L 711 from the company Union Carbide or the (C₁₋₂)alkylmethicone copolyol sold by the company Dow Corning under the name Q2 5200;
thiol groups, such as the products sold under the names GP 72 A and GP 71 from Genesee;
alkoxylated groups, such as the product sold under the name Silicone Copolymer F755 by SWS Silicones and Abil Wax 2428, 2434 and 2440 by the company Goldschmidt;
hydroxylated groups, such as the polyorganosiloxanes containing a hydroxyalkyl function, described in French patent application FR-A-85/16334;
acyloxyalkyl groups, for instance the polyorganosiloxanes described in U.S. Pat. No. 4,957,732;
anionic groups of the phosphate or carboxylic type, for instance in the products described in patent EP 186 507 from the company Chisso Corporation, or of the alkylcarboxylic type, such as those present in the product X-22-3701 E from the company Shin-Etsu; 2-hydroxyalkyl sulphonate; 2-hydroxyalkyl thiosulphate such as the products sold by the company Goldschmidt under the names Abil S201 and Abil S255.

Among the organomodified silicones, mention may also be made of amino silicones.

The term “amino silicone” means any polyaminosiloxane, i.e. any polysiloxane comprising at least one primary, secondary or tertiary amine function or a quaternary ammonium group.

Preferably, the amino silicone(s) used in the cosmetic composition according to the present invention are selected from:

(a) the compounds corresponding to formula (II) below:

(R¹)ₐ(T)₃₋ₐSi[OSi(T)₂]ₙ-[OSi(T)_{b}(R¹)_{2-b}]ₘ—OSi(T)₃₋ₐ-(R¹)ₐ (II)

in which:
T is a hydrogen atom or a phenyl, hydroxyl (—OH) or C₁-C₈ alkyl radical, and preferably methyl, or a C₁-C₈ alkoxy, preferably methoxy,
a denotes the number 0 or an integer from 1 to 3, and preferably 0,
b denotes 0 or 1, and in particular 1,
m and n are numbers such that the sum (n+m) can range especially from 1 to 2000 and in particular from 50 to 150, it being possible for n to denote a number from 0 to 1999 and in particular from 49 to 149, and for m to denote a number from 1 to 2000 and in particular from 1 to 10;
R¹ is a monovalent radical of formula —C_{q}H_{2q}L in which q is a number from 2 to 8 and L is an optionally quaternized amino group selected from the following groups:

### —N(R²)—CH₂—CH₂—N(R²)₂;

### —N(R²)₂; —N⁺(R²)₃Q⁻;

### —N⁺(R²)(H)₂Q⁻;

### —N⁺(R²)₂HQ⁻;

### —N(R²)—CH₂—CH₂—N⁺(R²)(H)₂Q⁻,

in which R² denotes a hydrogen atom, a phenyl, a benzyl or a saturated monovalent hydrocarbon-based radical, for example a C₁-C₂₀ alkyl radical, and Q⁻ represents a halide ion, for instance fluoride, chloride, bromide or iodide.

In particular, the amino silicones corresponding to the definition of formula (II) are selected from the compounds corresponding to formula (III) below:

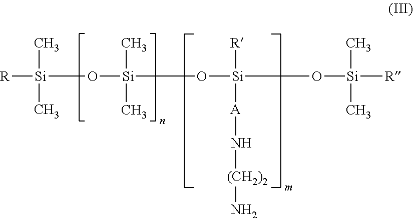

in which R, R′ and R″, which may be identical or different, denote a C₁-C₄ alkyl radical, preferably CH₃; a C₁-C₄ alkoxy radical, preferably methoxy; or OH; A represents a linear or branched, C₃-C₈ and preferably C₃-C₆ alkylene radical; m and n are integers dependent on the molecular weight and whose sum is between 1 and 2000.

According to a first possibility, R, R′, R″, which may be identical or different, represent a C₁-C₄ alkyl or hydroxyl radical, A represents a C₃ alkylene radical and m and n are such that the weight-average molecular weight of the compound is between 5000 and 500 000 approximately. Compounds of this type are referred to in the CTFA dictionary as “amodimethicones”.

According to a second possibility, R, R′ and R″, which may be identical or different, represent a C₁-C₄ alkoxy or hydroxyl radical, at least one of the radicals R or R″ is an alkoxy radical and A represents a C₃ alkylene radical. The hydroxy/alkoxy molar ratio is preferably between 0.2/1 and 0.4/1 and advantageously equal to 0.3/1. Moreover, m and n are such that the weight-average molecular weight of the compound is between 2000 and 10⁶. More particularly, n is between 0 and 999 and m is between 1 and 1000, the sum of n and m being between 1 and 1000.

In this category of compounds, mention may be made, inter alia, of the product Belsil® ADM 652 sold by Wacker.

According to a third possibility, R and R″, which are different, represent a C₁-C₄ alkoxy or hydroxyl radical, at least one of the radicals R or R″ is an alkoxy radical, R′ represents a methyl radical and A represents a C₃ alkylene radical. The hydroxy/alkoxy molar ratio is preferably between 1/0.8 and 1/1.1 and advantageously equal to 1/0.95. Moreover, m and n are such that the weight-average molecular weight of the compound is between 2000 and 200 000. More particularly, n is between 0 and 999 and m is between 1 and 1000, the sum of n and m being between 1 and 1000.

More particularly, mention may be made of the product Fluid WR® 1300 sold by Wacker.

According to a fourth possibility, R and R″ represent a hydroxyl radical, R′ represents a methyl radical and A is a C₄-C₈ and preferably C₄ alkylene radical.

Moreover, m and n are such that the weight-average molecular weight of the compound is between 2000 and 10⁶. More particularly, n is between 0 and 1999 and m is between 1 and 2000, the sum of n and m being between 1 and 2000.

A product of this type is especially sold under the name DC 28299 by Dow Corning.

Note that the molecular weight of these silicones is determined by gel permeation chromatography (ambient temperature, polystyrene standard; p styragem columns; eluent THF; flow rate 1 mm/m; 200 μl of a solution containing 0.5% by weight of silicone in THF are injected, and detection is performed using a refractometer and a UV meter).

A product corresponding to the definition of formula (I) is in particular the polymer known in the CTFA dictionary (7th edition, 1997) as “trimethylsilylamodimethicone”, corresponding to formula (IV) below:

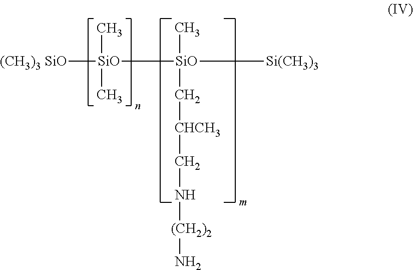

in which n and m have the meanings given above in accordance with formula (II) or (III).

Such compounds are described, for example, in EP 0 095 238; a compound of formula (IV) is sold, for example, under the name Q2-8220 by the company OSI.

(b) the compounds corresponding to formula (V) below:

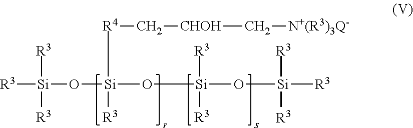

in which:
R³ represents a C₁-C₁₈ monovalent hydrocarbon-based radical, and in particular a C₁-C₁₈ alkyl or C₂-C₁₈ alkenyl radical, for example methyl;
R⁴ represents a divalent hydrocarbon-based radical, especially a C₁-C₁₈ alkylene radical or a divalent C₁-C₁₈, and for example C₁-C₈, alkylenoxy radical;
Q⁻ is a halide ion, in particular chloride;
r represents a mean statistical value from 2 to 20 and in particular from 2 to 8;
s represents a mean statistical value from 20 to 200 and in particular from 20 to 50.

Such compounds are described more particularly in U.S. Pat. No. 4,185,087.

A compound falling within this class is the product sold by the company Union Carbide under the name Ucar Silicone ALE 56.

(c) the quaternary ammonium silicones of formula (VI):

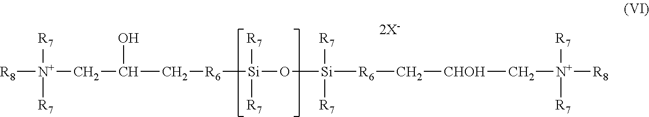

in which:
R₇, which may be identical or different, represent a monovalent hydrocarbon-based radical containing from 1 to 18 carbon atoms, and in particular a C₁-C₁₈ alkyl radical, a C₂-C₁₈ alkenyl radical or a ring comprising 5 or 6 carbon atoms, for example methyl;
R₆ represents a divalent hydrocarbon-based radical, especially a C₁-C₁₈ alkylene radical or a divalent C₁-C₁₈, and for example C₁-C₈, alkylenoxy radical linked to the Si via an SiC bond;
R₈, which may be identical or different, represent a hydrogen atom, a monovalent hydrocarbon-based radical containing from 1 to 18 carbon atoms, and in particular a C₁-C₁₈ alkyl radical, a C₂-C₁₈ alkenyl radical or a radical —R₆—NHCOR₇;
X⁻ is an anion such as a halide ion, especially chloride, or an organic acid salt (acetate, etc.);
r represents a mean statistical value from 2 to 200 and in particular from 5 to 100;

These silicones are described, for example, in patent application EP-A 0 530 974. Mention may be made, as the compound of formula (VI), of the product referenced in the CTFA dictionary (1997 edition) under the name Quaternium 80 as sold by the company Evonik Goldschmidt under the names ABIL QUAT 3272 or 3474.

d) the amino silicones of formula (VII) below:

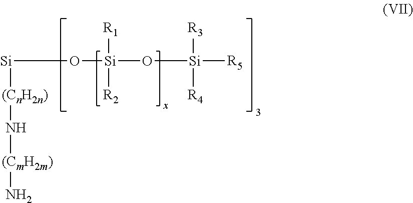

in which:
R₁, R₂, R₃ and R₄, which may be identical or different, denote a C₁-C₄ alkyl radical or a phenyl group,
R₅ denotes a C₁-C₄ alkyl radical or a hydroxyl group,
n is an integer ranging from 1 to 5,
m is an integer ranging from 1 to 5,
   and in which x is selected such that the amine number is between 0.01 and 1 meq/g.

The amino silicone(s) that are particularly preferred are polysiloxanes containing amine groups, such as the compounds of formula (III) or of formula (IV), and even more particularly the silicones containing quaternary ammonium groups of formula (VI).

When these compounds are used, one particularly advantageous embodiment involves their combined use with cationic and/or nonionic surfactants.

Use may be made, for example, of the product sold under the name Cationic Emulsion DC 939 by the company Dow Corning, which comprises, besides amodimethicone, a cationic surfactant which is trimethylcetylammonium chloride and a nonionic surfactant of formula: C₁₃H₂₇—(OC₂H₄)₁₂—OH, known under the CTFA name “trideceth-12”.

Another commercial product that may be used according to the invention is the product sold under the name Dow Corning Q2 7224 by the company Dow Corning, comprising, in combination, the trimethylsilyl amodimethicone of formula (C) described above, a nonionic surfactant of formula: C₈H₁₇—C₆H₄—(OCH₂CH₂)₄₀OH, known under the CTFA name “octoxynol-40”, a second nonionic surfactant of formula: C₁₂H₂₅—(OCH₂—CH₂)₆—OH, known under the CTFA name “isolaureth-6”, and propylene glycol.

Preferably, the organomodified silicones of the invention are amino silicones. The organomodified silicone(s) may be present in a content ranging from 0.01% to 15% by weight, preferably ranging from 0.1% to 10% by weight and more preferentially ranging from 0.2% to 5% by weight, with respect to the total weight of the composition of the invention.

The product(s) (i)/organomodified silicones ii) weight ratio ranges from 0.1 to 50, more preferably still from 1 to 25, better still from 1 to 10.

The composition according to the invention may comprise one or more cationic polymers.

The term “cationic polymer” means a polymer that is positively charged when it is contained in the composition according to the invention. This polymer may bear one or more positive permanent charges or may contain one or more cationizable functions in the composition according to the invention.

The cationic polymer(s) that may be used as conditioning agents according to the present invention are preferably selected from polymers comprising primary, secondary, tertiary and/or quaternary amine groups forming part of the polymer chain or directly attached thereto, and having a molecular weight of between 500 and about 5 000 000 and preferably between 1000 and 3 000 000.

Among the cationic polymers that may be mentioned more particularly are polymers of the polyamine, polyaminoamide and polyquaternary ammonium type.

These are known products. They are described, for example, in French patents 2 505 348 and 2 542 997.

Among these polymers, mention may be made of:

(1) homopolymers or copolymers that are derived from acrylic or methacrylic esters or amides and comprise at least one of the units of the following formulae:

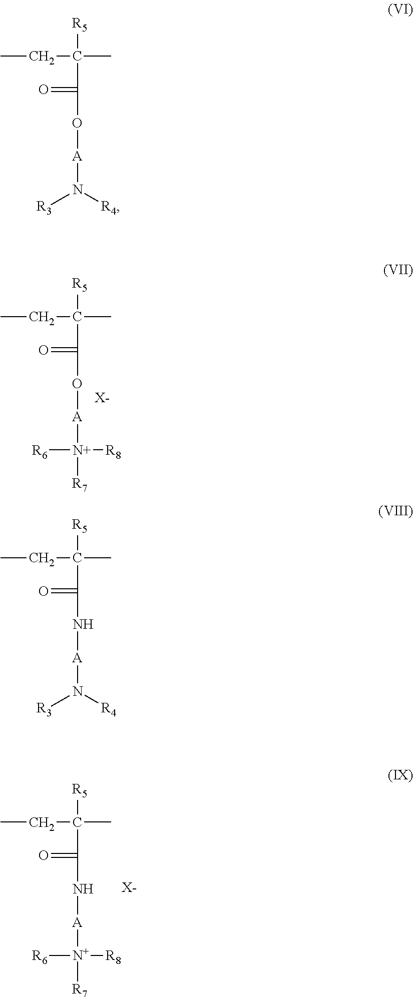

in which:
R₃ and R₄, which may be identical or different, represent hydrogen or an alkyl group containing from 1 to 6 carbon atoms and preferably methyl or ethyl;
R₅, which may be identical or different, denote a hydrogen atom or a CH₃ radical;
A, which may be identical or different, represent a linear or branched alkyl group of 1 to 6 carbon atoms, preferably 2 or 3 carbon atoms, or a hydroxyalkyl group of 1 to 4 carbon atoms;
R₆, R₇ and R₈, which may be identical or different, represent an alkyl group containing from 1 to 18 carbon atoms or a benzyl radical and preferably an alkyl group containing from 1 to 6 carbon atoms;
X denotes an anion derived from a mineral or organic acid, such as a methosulphate anion or a halide such as chloride or bromide.

The copolymers of family (1) can also contain one or more units derived from comonomers that may be selected from the family of acrylamides, methacrylamides, diacetone acrylamides, acrylamides and methacrylamides substituted on the nitrogen with lower (C₁-C₄) alkyls, acrylic or methacrylic acids or esters thereof, vinyllactams such as vinylpyrrolidone or vinylcaprolactam, and vinyl esters.

Thus, among these copolymers of the family (1), mention may be made of:
copolymers of acrylamide and of dimethylaminoethyl methacrylate quaternized with dimethyl sulphate or with a dimethyl halide, such as the product sold under the name Hercofloc by the company Hercules,
the copolymers of acrylamide and of methacryloyloxyethyltrimethylammonium chloride described, for example, in patent application EP-A-080 976 and sold under the name Bina Quat P 100 by the company Ciba Geigy,
the copolymer of acrylamide and of methacryloyloxyethyltrimethylammonium methosulphate sold under the name Reten by the company Hercules,
quaternized or non-quaternized vinylpyrrolidone/dialkylaminoalkyl acrylate or methacrylate copolymers, such as the products sold under the name Gafquat by the company ISP, such as, for example, Gafquat 734 or Gafquat 755, or alternatively the products known as Copolymer 845, 958 and 937. These polymers are described in detail in French Patents 2 077 143 and 2 393 573,
dimethylaminoethyl methacrylate/vinylcaprolactam/vinylpyrrolidone terpolymers, such as the product sold under the name Gaffix VC 713 by the company ISP,
vinylpyrrolidone/methacrylamidopropyldimethylamine copolymers sold in particular under the name Styleze CC 10 by ISP,
quaternized vinylpyrrolidone/dimethylaminopropylmethacrylamide copolymers such as the product sold under the name Gafquat HS 100 by the company ISP, and
crosslinked polymers of methacryloyloxy(C₁-C₄)alkyltri(C₁-C₄)alkylammonium salts, such as the polymers obtained by homopolymerization of dimethylaminoethyl methacrylate quaternized with methyl chloride, or by copolymerization of acrylamide with dimethylaminoethyl methacrylate quaternized with methyl chloride, the homopolymerization or copolymerization being followed by crosslinking with an olefinically unsaturated compound, more particularly methylenebisacrylamide. A crosslinked acrylamide/methacryloyloxyethyl-trimethyl-ammonium chloride copolymer (20/80 by weight) in the form of a dispersion containing 50% by weight of said copolymer in mineral oil can be used more particularly. This dispersion is sold under the name Salcare® SC 92 by the company Ciba. A crosslinked methacryloyloxyethyltrimethylammonium chloride homopolymer, for example as a dispersion in mineral oil or in a liquid ester, can also be used. These dispersions are sold under the names Salcare® SC 95 and Salcare® SC 96 by the company Ciba.
   (2) Polymers formed from piperazinyl units and divalent alkylene or hydroxyalkylene radicals containing straight or branched chains, optionally interrupted with oxygen, sulphur or nitrogen atoms or with aromatic or heterocyclic rings, and also the oxidation and/or quaternization products of these polymers. Such polymers are especially described in French Patents 2 162 025 and 2 280 361.
   (3) Water-soluble polyaminoamides prepared in particular by polycondensation of an acidic compound with a polyamine; these polyaminoamides can be crosslinked with an epihalohydrin, a diepoxide, a dianhydride, an unsaturated dianhydride, a bisunsaturated derivative, a bis-halohydrin, a bisazetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyaminoamide; these polyaminoamides can be alkylated or, if they comprise one or more tertiary amine functions, they can be quaternized. Such polymers are especially described in French Patents 2 252 840 and 2 368 508.
   (4) polyaminoamide derivatives resulting from the condensation of polyalkylene polyamines with polycarboxylic acids followed by alkylation with difunctional agents. Mention may be made, for example, of adipic acid/dialkylaminohydroxy-alkyldialkylenetriamine polymers in which the alkyl radical comprises from 1 to 4 carbon atoms and preferably denotes methyl, ethyl or propyl. Such polymers are especially described in French patent 1 583 363.

Among these derivatives, mention may be made more particularly of the adipic acid/dimethylaminohydroxypropyl/diethylenetriamine polymers sold under the name Cartaretine F, F4 or F8 by the company Sandoz.

(5) The polymers obtained by reaction of a polyalkylene polyamine containing two primary amine groups and at least one secondary amine group with a dicarboxylic acid selected from diglycolic acid and saturated aliphatic dicarboxylic acids having from 3 to 8 carbon atoms. The molar ratio between the polyalkylene polyamine and the dicarboxylic acid is between 0.8:1 and 1.4:1; the polyaminoamide resulting therefrom is reacted with epichlorohydrin in a molar ratio of epichlorohydrin relative to the secondary amine group of the polyaminoamide of between 0.5:1 and 1.8:1. Such polymers are described in particular in U.S. Pat. Nos. 3,227,615 and 2,961,347.

Polymers of this type are sold in particular under the name Hercosett 57 by the company Hercules Inc. or alternatively under the name PD 170 or Delsette 101 by the company Hercules in the case of the adipic acid/epoxypropyl/diethylenetriamine copolymer.

(6) Cyclopolymers of alkyldiallylamine or of dialkyldiallylammonium, such as the homopolymers or copolymers containing, as main constituent of the chain, units corresponding to formula (X) or (XI):

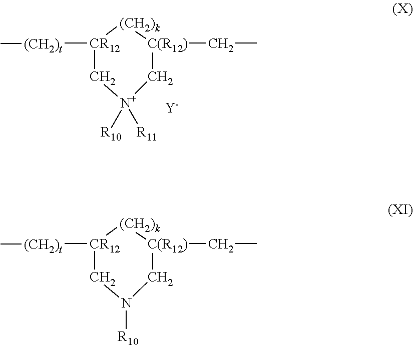

in which formulae k and t are equal to 0 or 1, the sum k+t being equal to 1; R₁₂ denotes a hydrogen atom or a methyl radical; R₁₀ and R₁₁, independently of one another, denote an alkyl group having from 1 to 6 carbon atoms, a hydroxyalkyl group in which the alkyl group has preferably 1 to 5 carbon atoms, a lower (C₁-C₄) amidoalkyl group, or else R₁₀ and R₁₁ may, together with the nitrogen atom to which they are attached, denote heterocyclic groups, such as piperidyl or morpholinyl; r is an anion such as bromide, chloride, acetate, borate, citrate, tartrate, bisulphate, bisulphite, sulphate or phosphate. These polymers are especially described in French Patent 2 080 759 and in its Certificate of Addition 2 190 406.
R₁₀ and R₁₁, independently of one another, preferably denote an alkyl group having from 1 to 4 carbon atoms.

Among the polymers defined above, mention may be made more particularly of the dimethyldiallylammonium chloride homopolymer sold under the name Merquat 100 by the company Nalco (and its homologues of low weight-average molecular weights) and the copolymers of diallyldimethylammonium chloride and of acrylamide, sold under the name Merquat 550.

(7) The quaternary diammonium polymer containing repeating units corresponding to the formula:

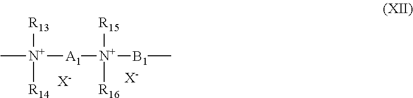

in which formula (XII):
R₁₃, R₁₄, R₁₅ and R₁₆, which may be identical or different, represent aliphatic, alicyclic or arylaliphatic radicals containing from 1 to 20 carbon atoms, or lower hydroxyalkylaliphatic radicals, or else R₁₃, R₁₄, R₁₅ and R₁₆, together or separately, constitute, with the nitrogen atoms to which they are attached, heterocycles optionally containing a second heteroatom other than the nitrogen, or else R₁₃, R₁₄, R₁₅ and R₁₆ represent a linear or branched C₁-C₆ alkyl radical which is substituted by a nitrile, ester, acyl, amide or CO—O—R₁₇-D or —CO—NH—R₁₇-D group in which R₁₇ is an alkylene and D is a quaternary ammonium group;
A₁ and B₁ represent polymethylene groups containing from 2 to 20 carbon atoms, which may be linear or branched, saturated or unsaturated, and which may contain, linked to or intercalated in the main chain, one or more aromatic rings or one or more oxygen or sulphur atoms or sulphoxide, sulphone, disulphide, amino, alkylamino, hydroxyl, quaternary ammonium, ureido, amide or ester groups, and
X⁻ denotes an anion derived from a mineral or organic acid;
A₁, R₁₃ and R₁₅ may form, with the two nitrogen atoms to which they are attached, a piperazine ring; moreover, if A₁ denotes a saturated or unsaturated, linear or branched alkylene or hydroxyalkylene radical, B₁ may also denote a group (CH₂)ₙ—CO-D—OC—(CH₂)ₚ—
   n and p are integers ranging from 2 to 20 approximately
in which D denotes:
a) a glycol residue of formula: where Z denotes a linear or branched hydrocarbon-based radical or a group corresponding to one of the following formulae:

—(CH₂—CH₂—O)x—CH₂—CH₂—

—[CH₂—CH(CH₃)—O]y-CH₂—CH(CH₃)—
where x and y denote an integer from 1 to 4, representing a defined and unique degree of polymerization or any number from 1 to 4 representing an average degree of polymerization;
b) a bis-secondary diamine residue such as a piperazine derivative;
c) a bis-primary diamine residue of formula: —NH—Y—NH—, where Y denotes a linear or branched hydrocarbon-based radical, or alternatively the divalent radical

—CH₂—CH₂—S—S—CH₂—CH₂—;
d) a ureylene group of formula: —NH—CO—NH—.
Preferably, X⁻ is an anion such as chloride or bromide.
These polymers generally have a number-average molecular weight of between 1000 and 100 000.
Polymers of this type are described in particular in French Patents 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

Use may be made more particularly of polymers that are composed of repeating units corresponding to the formula:

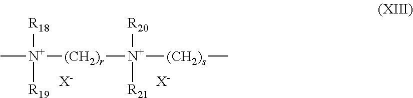

in which R₁₈, R₁₉, R₂₀ and R₂₁, which may be identical or different, denote an alkyl or hydroxyalkyl radical having from about 1 to 4 carbon atoms, r and s are integers varying from 2 to 20 approximately, and X⁻ is an anion derived from a mineral or organic acid.

One particularly preferred compound of formula (XIII) is that for which R₁₈, R₁₉, R₂₀ and R₂₁ represent a methyl radical and r=3, s=6 and X=Cl, which is called Hexadimethrine chloride according to INCI nomenclature (CTFA).

(8) Polyquaternary ammonium polymers composed of units of formula (XIV):

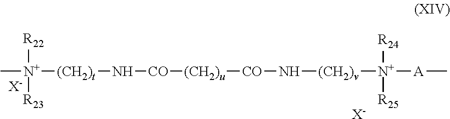

in which formula:
R₂₂, R₂₃, R₂₄ and R₂₅, which may be identical or different, represent a hydrogen atom or a methyl, ethyl, propyl, β-hydroxyethyl, β-hydroxypropyl or —CH₂CH₂(OCH₂CH₂)pOH radical,
in which p is equal to 0 or to an integer between 1 and 6, with the proviso that R₂₂, R₂₃, R₂₄ and R₂₅ do not simultaneously represent a hydrogen atom,
t and u, which are identical or different, are integers between 1 and 6,
v is equal to 0 or to an integer between 1 and 34,
X⁻ denotes an anion such as a halide,
A denotes a radical of a dihalide or represents preferably —CH₂—CH₂—O—CH₂—CH₂—.

Such compounds are described especially in patent application EP-A-122 324.

Among these, mention may be made, for example, of the products Mirapol® A 15, Mirapol® AD1, Mirapol® AZ1 and Mirapol® 175, sold by the company Miranol.

(9) Quaternary polymers of vinylpyrrolidone and of vinylimidazole, for instance the products sold under the names Luviquat® FC 905, FC 550 and FC 370 by the company BASF.
(10) Cationic polysaccharides, especially cationic celluloses and galactomannan gums.

Among cationic polysaccharides, mention may be made more particularly of cellulose ether derivatives comprising quaternary ammonium groups, cationic cellulose copolymers or cellulose derivatives grafted with a water-soluble quaternary ammonium monomer and cationic galactomannan gums.

The cellulose ether derivatives comprising quaternary ammonium groups described in French Patent 1 492 597. These polymers are also defined in the CTFA dictionary as quaternary ammoniums of hydroxyethyl cellulose that have reacted with an epoxide substituted with a trimethylammonium group.

Cationic cellulose copolymers or cellulose derivatives grafted with a water-soluble monomer of quaternary ammonium are described especially in U.S. Pat. No. 4,131,576, such as hydroxyalkyl celluloses, for instance hydroxymethyl-, hydroxyethyl- or hydroxypropylcelluloses grafted, in particular, with a methacryloylethyltrimethylammonium, methacrylamidopropyltrimethylammonium or dimethyldiallylammonium salt.

The cationic galactomannan gums are described more particularly in U.S. Pat. Nos. 3,589,578 and 4,031,307, in particular guar gums containing cationic trialkylammonium groups. Guar gums modified with a salt (e.g. chloride) of 2,3-epoxypropyltrimethylammonium are used, for example.

Other cationic polymers that may be used in the context of the invention are cationic proteins or cationic protein hydrolysates, polyalkyleneimines, in particular polyethyleneimines, polymers containing vinylpyridine or vinylpyridinium units, condensates of polyamines and of epichlorohydrin, quaternary polyureylenes and chitin derivatives.

The cationic proteins or protein hydrolysates are, in particular, chemically modified polypeptides bearing quaternary ammonium groups at the end of the chain, or grafted thereon. Their molecular weight may vary, for example, from 1500 to 10 000 and in particular from 2000 to 5000 approximately. Among these compounds, mention may be made especially of:
collagen hydrolysates carrying triethylammonium groups, such as the products sold under the name Quat-Pro E by the company Maybrook and referred to in the CTFA dictionary as Triethonium Hydrolyzed Collagen Ethosulphate;
collagen hydrolysates bearing trimethylammonium chloride and trimethylstearylammonium chloride groups, which are sold under the name Quat-Pro S by the company Maybrook and are referred to in the CTFA dictionary as Steartrimonium Hydrolyzed Collagen;
animal protein hydrolysates which bear trimethylbenzylammonium groups, such as the products sold under the name Crotein BTA by the company Croda and referred to in the CTFA dictionary as Benzyltrimonium hydrolyzed animal protein;
protein hydrolysates bearing quaternary ammonium groups on the polypeptide chain, said ammonium groups containing at least one alkyl radical having from 1 to 18 carbon atoms.

Among these protein hydrolysates, mention may be made, inter alia, of:
Croquat L, in which the quaternary ammonium groups contain a C12 alkyl group;
Croquat M, in which the quaternary ammonium groups contain C10-C18 alkyl groups;
Croquat S, in which the quaternary ammonium groups contain a C18 alkyl group;
Crotein Q, in which the quaternary ammonium groups contain at least one alkyl group having from 1 to 18 carbon atoms.

These various products are sold by the company Croda.

Other quaternized proteins or hydrolysates are, for example, those corresponding to the formula (XV):

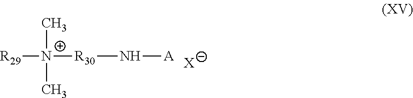

in which X⁻ is an anion of an organic or inorganic acid, A denotes a protein residue derived from collagen protein hydrolysates, R₂₉ denotes a lipophilic group containing up to 30 carbon atoms, R₃₀ represents an alkylene group having 1 to 6 carbon atoms. Mention may be made, for example, of the products sold by the company Inolex, under the name Lexein QX 3000, referred to, in the CTFA dictionary, as Cocotrimonium Collagen Hydrolysate.

Mention may also be made of quaternized plant proteins such as wheat, corn or soybean proteins: quaternized wheat proteins that may be mentioned include those sold by the company Croda under the names Hydrotriticum WQ or QM, which in the CTFA dictionary are called Cocodimonium Hydrolysed wheat protein, or Hydrotriticum QL, which in the CTFA dictionary is called Laurdimonium hydrolysed wheat protein, or else Hydrotriticum QS, which in the CTFA dictionary is called Steardimonium hydrolysed wheat protein.

Among all the cationic polymers that can be used in the context of the present invention, it is preferred to use cationic cyclopolymers, in particular the dimethyldiallylammonium chloride homopolymers or copolymers sold under the names Merquat 100, Merquat 550 and Merquat S by the company Nalco, and quaternary vinylpyrrolidone and vinylimidazole polymers, cationic polysaccharides, more particularly guar gums modified by 2,3-epoxypropyltrimethylammonium chloride that are sold, for example, under the name Jaguar C13S by the company RHODIA or derivatives of cellulose ethers comprising quaternary ammonium groups such as the cellulose ethers sold, for example, under the name Ucare Polymer JR400LT by the company Amerchol (Dow Chemical), and mixtures thereof.

The cationic polymer or polymers ii) can be present in a content ranging from 0.005% to 15% by weight, preferably ranging from 0.01% to 10% by weight and more preferentially ranging from 0.1% to 5% by weight, with respect to the total weight of the composition of the invention.

The product(s) (i)/cationic polymer ii) weight ratio preferably ranges from 0.1 to 50, more preferably still from 1 to 25, better still from 10 to 25.

The composition according to the invention may comprise one or more particular cationic surfactants.

Generally, a cationic surfactant can carry one or more permanent positive charges or can comprise one or more functional groups which can form cations in the composition according to the invention. The cationic surfactants of the invention are those carrying one or more permanent positive charges, that is to say one or more quaternized nitrogen atoms.

Mention may in particular be made, as example of cationic surfactants which can be used in the cosmetic composition according to the invention, of:
quaternary ammonium salts, such as the quaternary ammonium salts which have the general formula (II) below:

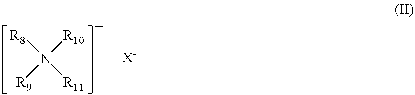

in which the radicals R₈ to R₁₁, which may be identical or different, represent a linear or branched aliphatic radical comprising from 1 to 30 carbon atoms or an aromatic radical such as aryl or alkylaryl, at least one of the radicals R₈ to R₁₁ comprising from 8 to 30 carbon atoms and preferably from 12 to 24 carbon atoms. The aliphatic radicals may comprise heteroatoms especially such as oxygen, nitrogen, sulphur and halogens.

The aliphatic radicals are for example selected from alkyl, alkoxy, polyoxy(C₂-C₆)alkylene, alkylamide, (C₁₂-C₂₂)alkylamido(C₂-C₆)alkyl, (C₁₂-C₂₂)alkyl acetate and hydroxyalkyl radicals containing approximately from 1 to 30 carbon atoms; X⁻ is an anion selected from the group of halides, phosphates, acetates, lactates, (C₂-C₆)alkyl sulphates, alkyl sulphonates or alkylaryl sulphonates;
quaternary ammonium salts of imidazoline, for instance those of formula (III) below:

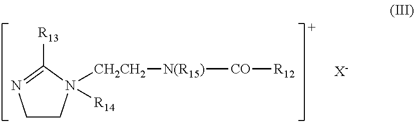

in which R₁₂ represents an alkenyl or alkyl radical containing from 8 to 30 carbon atoms, for example tallow fatty acid derivatives, R₁₃ represents a hydrogen atom, a C₁-C₄ alkyl radical or an alkenyl or alkyl radical containing from 8 to 30 carbon atoms, R₁₄ represents a C₁-C₄ alkyl radical, R₁₅ represents a hydrogen atom or a C₁-C₄ alkyl radical, X⁻ is an anion selected from the group of halides, phosphates, acetates, lactates, alkyl sulphates, alkyl sulphonates or alkylaryl sulphonates. Preferably, R₁₂ and R₁₃ denote a mixture of alkenyl or alkyl radicals comprising from 12 to 21 carbon atoms, for example fatty acid derivatives of tallow, R₁₄ denotes a methyl radical and R₁₅ denotes a hydrogen atom. Such a product is sold, for example, under the name Rewoquat® W 75 by the company Rewo;

Among the quaternary ammonium salts of formula (II), those that are preferred are, on the one hand, tetraalkylammonium chlorides, for instance dialkyldimethyl-ammonium or alkyltrimethylammonium chlorides in which the alkyl radical contains approximately from 12 to 22 carbon atoms, in particular behenyltrimethyl-ammonium, distearyldimethylammonium, cetyltrimethylammonium or benzyl-dimethylstearylammonium chlorides, or else, on the other hand, distearoylethyl hydroxyethylmethylammonium methosulphate, dipalmitoylethylhydroxyethyl-ammonium methosulphate or distearoylethylhydroxyethylammonium methosulphate, or else, finally, palmitylamidopropyltrimethylammonium chloride or stearamidopropyldimethyl(myristyl acetate)ammonium chloride sold under the name Ceraphyl® 70 by the company Van Dyk.
quaternary di- or triammonium salts in particular of formula (IV):

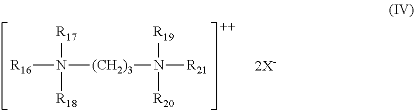

in which R₁₆ denotes an alkyl radical containing approximately from 16 to 30 carbon atoms, which is optionally hydroxylated and/or interrupted with one or more oxygen atoms, R₁₇ is selected from hydrogen or an alkyl radical containing from 1 to 4 carbon atoms or a group (R₁₆ₐ)(R₁₇ₐ)(R₁₈ₐ)N⁺(CH₂)₃⁻, R₁₆ₐ, R₁₇ₐ, R₁₈ₐ, R₁₈, R₁₉, R₂₀ and R₂₁, which may be identical or different, are selected from hydrogen or an alkyl radical containing from 1 to 4 carbon atoms, and X⁻ is an anion selected from the group of halides, acetates, phosphates, nitrates and (C₁-C₄)alkyl sulphates. Such compounds are, for example, Finquat CT-P available from the company Finetex (Quaternium 89), and Finquat CT, available from the company Finetex (Quaternium 75),
quaternary ammonium salts containing at least one ester function, such as those of formula (V) below:

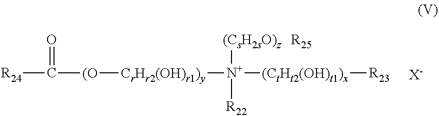

in which:
R₂₂ is selected from C₁-C₆ alkyl radicals and C₁-C₆ hydroxyalkyl or dihydroxyalkyl radicals;
R₂₃ is selected from:
the

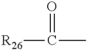

radicals R₂₇, which are linear or branched, saturated or unsaturated C₁-C₂₂ hydrocarbon-based radicals,
a hydrogen atom,
   R₂₅ is selected from:
the

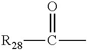

radicals R₂₉, which are linear or branched, saturated or unsaturated C₁-C₆ hydrocarbon-based radicals,
a hydrogen atom,
   R₂₄, R₂₆ and R₂₈, which may be identical or different, are selected from linear or branched, saturated or unsaturated C₇-C₂₁ hydrocarbon-based radicals;
   r, s and t, which may be identical or different, are integers ranging from 2 to 6;
   r1 and t1, which may be identical or different, are equal to 0 or 1;
   r2+r1=2r and t1+t2=2t
   y is an integer ranging from 1 to 10;
   x and z, which may be identical or different, are integers ranging from 0 to 10;
   X⁻ is a simple or complex, organic or inorganic anion; with the proviso that the sum x+y+z is from 1 to 15, that when x is 0, then R₂₃ denotes R₂₇ and that when z is 0, then R₂₅ denotes R₂₉.

The alkyl radicals R₂₂ may be linear or branched, and more particularly linear.

Preferably, R₂₂ denotes a methyl, ethyl, hydroxyethyl or dihydroxypropyl radical, and more particularly a methyl or ethyl radical.

Advantageously, the sum x+y+z is from 1 to 10.

When R₂₃ is a hydrocarbon-based radical R₂₇, it may be long and may contain from 12 to 22 carbon atoms, or may be short and may contain from 1 to 3 carbon atoms.

When R₂₅ is a hydrocarbon-based radical R₂₉, it preferably contains 1 to 3 carbon atoms.

Advantageously, R₂₄, R₂₆ and R₂₈, which may be identical or different, are selected from linear or branched, saturated or unsaturated C₁₁-C₂₁ hydrocarbon-based radicals, and more particularly from linear or branched, saturated or unsaturated C₁₁-C₂₁ alkyl and alkenyl radicals.

Preferably, x and z, which may be identical or different, are equal to 0 or 1.

Advantageously, y is equal to 1.

Preferably, r, s and t, which may be identical or different, are equal to 2 or 3, and even more particularly are equal to 2.

The anion X⁻ is preferably a halide (chloride, bromide or iodide) or an alkyl sulphate, more particularly methyl sulphate. However, use may be made of methanesulphonate, phosphate, nitrate, tosylate, an anion derived from an organic acid, such as acetate or lactate, or any other anion compatible with the ammonium containing an ester function.

The anion X⁻ is even more particularly chloride or methyl sulphate.

Use is made more particularly in the composition according to the invention of the ammonium salts of formula (V) in which:
R₂₂ denotes a methyl or ethyl radical,
x and y are equal to 1;
z is equal to 0 or 1;
r, s and t are equal to 2;
R₂₃ is selected from:
the

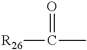

methyl, ethyl or C₁₄-C₂₂ hydrocarbon-based radicals,
a hydrogen atom;
R₂₅ is selected from:
the

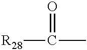

a hydrogen atom;
R₂₄, R₂₆ and R₂₈, which may be identical or different, are selected from linear or branched, saturated or unsaturated C₁₃-C₁₇ hydrocarbon-based radicals, and preferably from linear or branched, saturated or unsaturated C₁₃-C₁₇ alkyl and alkenyl radicals.

The hydrocarbon-based radicals are advantageously linear.

Mention may be made, for example, of the compounds of formula (V) such as diacyloxyethyldimethylammonium, diacyloxyethylhydroxyethylmethylammonium, monoacyloxyethyldihydroxyethylmethylammonium triacyloxyethylmethylammonium or monoacyloxyethylhydroxyethyldimethylammonium salts (chloride or methyl sulphate, in particular), and mixtures thereof. The acyl radicals preferably contain 14 to 18 carbon atoms and are obtained more particularly from a plant oil such as palm oil or sunflower oil. When the compound contains several acyl radicals, these radicals may be identical or different.

These products are obtained, for example, by direct esterification of triethanolamine, triisopropanolamine, alkyldiethanolamine or alkyldiisopropanolamine, which are optionally oxyalkylenated, with fatty acids or with fatty acid mixtures of plant or animal origin, or by transesterification of the methyl esters thereof. This esterification is followed by a quaternization by means of an alkylating agent such as an alkyl (preferably methyl or ethyl)halide, a dialkyl (preferably methyl or ethyl)sulphate, methyl methanesulphonate, methyl para-toluenesulphonate, glycol chlorohydrin or glycerol chlorohydrin.

Such compounds are, for example, sold under the names Dehyquart® by Henkel, Stepanquat® by Stepan, Noxamium® by Ceca or Rewoquat® WE 18 by Rewo-Witco.

The composition according to the invention preferably contains a mixture of quaternary ammonium salts of mono-, di- and triesters with a weight majority of diester salts.

Examples of mixtures of ammonium salts that may be used include the mixture containing 15% to 30% by weight of acyloxyethyldihydroxyethylmethylammonium methyl sulphate, 45% to 60% of diacyloxyethylhydroxyethylmethylammonium methyl sulphate and 15% to 30% of triacyloxyethylmethylammonium methyl sulphate, the acyl radicals containing from 14 to 18 carbon atoms and being derived from palm oil that is optionally partially hydrogenated.

It is also possible to use the ammonium salts containing at least one ester function that are described in U.S. Pat. No. 4,874,554 and U.S. Pat. No. 4,137,180.

Use may especially be made of behenoylhydroxypropyltrimethylammonium chloride sold by KAO under the name Quartamin BTC 131.

Preferably, the ammonium salts containing at least one ester function contain two ester functions.

Among the quaternary ammonium salts of formula (I), those that are preferred are, on the one hand, tetraalkylammonium chlorides, for instance dialkyldimethyl-ammonium or alkyltrimethylammonium chlorides in which the alkyl radical contains approximately from 12 to 22 carbon atoms, in particular behenyltrimethyl-ammonium, distearyldimethylammonium, cetyltrimethylammonium or benzyl-dimethylstearylammonium chlorides, or else, on the other hand, distearoylethyl-hydroxyethylmethylammonium methosulphate, dipalmitoylethylhydroxyethyl-ammonium methosulphate or distearoylethylhydroxyethylammonium methosulphate, or else, finally, palmitylamidopropyltrimethylammonium chloride or stearamidopropyldimethyl(myristyl acetate)ammonium chloride sold under the name Ceraphyl® 70 by the company Van Dyk.

Among all of the cationic surfactants that may be present in the composition according to the invention, it is preferred to select cetyltrimethylammonium, behenyltrimethylammonium, dipalmitoylethylhydroxyethylmethylammonium, distearoylethylhydroxyethylmethylammonium and methyl(C₉-C₁₉)alkyl(C₁₀-C₂₀-alkylamidoethylimidazolium salts, stearamidopropyldimethylammonium salt, and mixtures thereof.

The cationic surfactant(s) ii) can be present in a content ranging from 0.01% to 15% by weight, preferably ranging from 0.1% to 10% by weight and more preferentially ranging from 0.2% to 5% by weight, with respect to the total weight of the composition of the invention.

The product(s) (i)/cationic surfactant ii) weight ratio ranges from 0.1 to 50, more preferably still from 1 to 25, better still from 1 to 10.

The composition according to the present invention may comprise one or more sulphate or sulphonate surfactants (ii).

The sulphate or sulphonate anionic surfactant(s) (ii) used in the compositions according to the invention are anionic surfactants comprising one or more sulphate functions (—OSO₃H or —OSO₃⁻) and/or one or more sulphonate functions (—SO₃H or —SO₃⁻).

When the sulphate or sulphonate anionic surfactants (ii) that may be used, alone or as mixtures, in the context of the present invention are in the form of salt(s), they may be selected from alkali metal salts, especially sodium or potassium salts and preferably sodium salts, ammonium salts, amine salts, amino alcohol salts or alkaline-earth metal salts such as magnesium salts, alkyl sulphates, alkylamido sulphates, alkyl ether sulphates, alkylamido ether sulphates, alkylaryl ether sulphates, alkyl ether sulphosuccinates, acyl isethionates, methyl acyl taurates; the alkyl or acyl radical of all these various compounds preferably containing from 8 to 24 carbon atoms, and the aryl radical preferably denoting a phenyl or benzyl group.

The average number of ethylene oxide or propylene oxide groups may especially range from 2 to 50 and more particularly from 2 to 10, better still from 2 to 5.

An anionic surfactant selected from sodium, triethanolamine, magnesium or ammonium (C₁₂-C₁₄)alkyl sulphates, oxyethylenated sodium, ammonium or magnesium (C₁₂-C₁₄)alkyl ether sulphates, sodium cocoyl isethionate and methyl acyl taurates is preferably used.

Preferably the sulphate or sulphonate anionic surfactants (ii) are selected from sodium (C₁₂-C₁₄)alkyl ether sulphates, in particular sodium lauryl ether sulphate, more preferably still those comprising from 2 to 3 mol of ethylene oxide.

The sulphate or sulphonate anionic surfactants (ii) are generally present in a content ranging from 0.1% to 50% by weight, preferably in a content ranging from 3% to 30% by weight and more preferentially in a content ranging from 5% to 20% by weight relative to the total weight of the cosmetic composition.

Preferably, the product(s) (i)/anionic surfactant ii) weight ratio ranges from 0.05 to 10, more preferably still from 0.1 to 5, better still from 0.25 to 1.

The composition according to the invention may comprise antidandruff agents, which may be selected from:

1) pyridinethione salts, especially the calcium, magnesium, barium, strontium, zinc, cadmium, tin and zirconium salts. The zinc salt of pyridinethione is particularly preferred.

The zinc salt of pyridinethione is sold especially under the name Omadine zinc by the company Arch Personal Care;

2) 1-hydroxy-2-pyrrolidone derivatives represented especially by formula (IV);

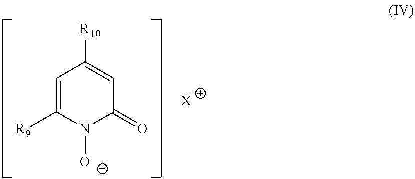

in which R₉ represents an alkyl group containing from 1 to 17 carbon atoms, an alkenyl group containing from 2 to 17 carbon atoms, a cycloalkyl group containing from 5 to 8 carbon atoms, a bicycloalkyl group containing from 7 to 9 carbon atoms; a cycloalkyl (-alkyl) group, an aryl group, an aralkyl group with an alkyl containing from 1 to 4 carbon atoms, an arylalkenyl group with an alkenyl containing from 2 to 4 carbon atoms, aryloxyalkyl or arylmercaptoalkyl with an alkyl containing from 1 to 4 carbon atoms, a furylalkenyl group with an alkenyl or a furyl containing from 2 to 4 carbon atoms, an alkoxy group containing from 1 to 4 carbon atoms, a nitro group, a cyano group or a halogen atom;
R₁₀ represents a hydrogen atom, a C₁-C₄ alkyl group, a C₂-C₄ alkenyl group, a halogen atom, a phenyl group, a benzyl group; Y represents an organic base, an alkali metal or alkaline-earth metal ion or an ammonium ion.

Examples of compounds of formula (IV) include 1-hydroxy-4-methyl-2-pyridone, 1-hydroxy-6-methyl-2-pyridone, 1-hydroxy-4,6-dimethyl-2-pyridone, 1-hydroxy-4-methyl-6-(2,4,4-trimethylpentyl)-2-pyridone, 1-hydroxy-4-methyl-6-cyclohexyl-2-pyridone, 1-hydroxy-4-methyl-6-(methylcyclohexyl)-2-pyridone, 1-hydroxy-4-methyl-6-(2-bicyclo[2,2,1]heptyl)-2-pyridone, 1-hydroxy-4-methyl-6-(4-methylphenyl)-2-pyridone, 1-hydroxy-4-methyl-6-[1-(4-nitrophenoxy)butyl]-2-pyridone, 1-hydroxy-4-methyl-6-(4-cyanophenoxymethyl)-2-pyridone, 1-hydroxy-4-methyl-6-(phenylsul-phonyl-methyl)-2-pyridone and 1-hydroxy-4-methyl-6-(4-bromobenzyl)-2-pyridone.

The compounds of formula (IV) may be used in the form of salts with organic or inorganic bases.

Examples of organic bases are especially alkanolamines of low molecular weight such as ethanolamine, diethanolamine, N-ethylethanolamine, triethanolamine, diethylaminoethanol and 2-amino-2-methylpropanediol; non-volatile bases such as ethylenediamine, hexamethylenediamine, cyclohexylamine, benzylamine and N-methylpiperazine; quaternary ammonium hydroxides, e.g. trimethylbenzyl hydroxide; guanidine and derivatives thereof, and particularly alkyl derivatives thereof. Examples of inorganic bases are especially salts of alkali metals, e.g. sodium or potassium; ammonium salts, salts of an alkaline-earth metal, e.g. magnesium or calcium; salts of di-, tri- or tetravalent cationic metals, e.g. zinc, aluminium or zirconium. Alkanolamines, ethylenediamine and inorganic bases such as alkali metal salts are preferred.

A compound of formula (IV) that is particularly preferred is the one for which:

R₉ denotes the radical

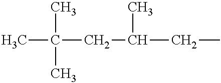

R₁₀ denotes methyl
and X+ denotes N+H₃CH₂CH₂OH

This compound is sold, for example, under the name Octopyrox (1-hydroxy-4-methyl-6-(2,4,4-trimethylpentyl)-2-pyridone, monoethanolamine salt) by the company Hoechst.

3) 2,2′-dithiobis(pyridine N-oxide) of formula (V):

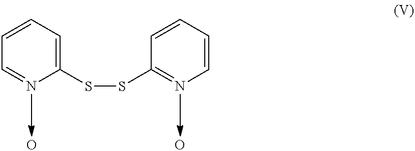 [SMILES: c1ccc(SSc2ccccn2)nc1 ; O ; O]

The compounds of formula (V) may be introduced into the compositions in the form of inorganic salts. An example of an inorganic salt is magnesium sulphate.

4) the trihalocarbamides especially of formula (VI) below:

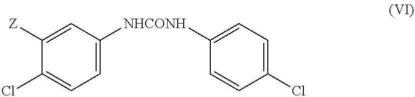

in which Z represents a halogen atom such as chlorine or a C₁-C₄ trihaloalkyl group such as CF₃.
5) triclosan, represented by formula (VII):

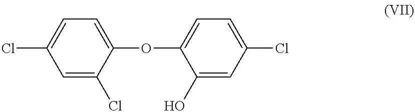 [SMILES: Oc1cc(Cl)ccc1Oc1ccc(Cl)cc1Cl]

6) azole compounds such as climbazole, ketoconazole, clotrimazole, econazole, isoconazole and miconazole.
7) selenium sulphides, in particular those of formula SₓSe₈₋ₓ, x ranging from 1 to 7.
8) extracts of one or more non-photosynthetic, non-fruiting filamentous bacteria. The bacterial extracts that may be used according to the invention will be selected from non-photosynthetic, non-fruiting filamentous bacteria as defined according to the classification in Bergey's Manual of Systemic Bacteriology, volume 3, section 23, 9th edition 1989.

Among the bacteria that may be used, mention will be made more particularly of bacteria belonging to the order Beggiatoales, and especially bacteria belonging to the genus Beggiotoa, for instance various strains of Beggiotoa alba. According to the definition, B. alba corresponds to the former names Beggiotoa arachnoidea, B. gigantea, B. leptomiformis, B. minima and B. mirabilis of Bergey's manual, 8th edition. Mention may moreover be made of bacteria belonging to the genus Vitreoscilla, which is known to be close to and often difficult to distinguish from the genus Beggiatoa. The bacteria that have just been defined, and several of which have been described, generally have an aquatic habitat, and may be found especially in spring water sources.

Among the bacteria that may be used, mention may be made, for example, of Vitreoscilla beggiatoides (ATCC 43181) and Beggiatoa alba (ATCC33555).

Preferentially, according to the invention, the use of the extract of Vitreoscilla filiformis, in particular the strain ATCC 15551, metabolites thereof and fractions thereof, are claimed.

Use will preferentially be made of the culture described in patent application WO 94/02158.

The term “non-photosynthetic, non-fruiting filamentous bacteria” means not only the culture supernatant but also the biomass obtained after culturing said bacteria, the envelopes or envelope fractions, or the extracts of the biomass obtained by treating this biomass.

To prepare the extract according to the invention, said bacteria can be cultured and then separated from the biomass obtained, for example by filtration, centrifugation, coagulation and/or lyophilization.

The extracts that may be used may especially be prepared according to the process described in patent application WO-A-93/00741. Thus, after culturing, the bacteria are concentrated by centrifugation. The biomass obtained is autoclaved.

This biomass may be lyophilized to constitute what is known as the lyophilized extract. Any lyophilization method known to those skilled in the art may be used to prepare this extract.

The supernatant fraction of this biomass may also be filtered in a sterile container to remove the particles in suspension.

The terms “envelopes” and “envelope fractions” refer herein to the bacterial wall and possibly the subjacent membranes.

Preferably, the antidandruff agents of the invention contain one or more sulphur atoms in their structure.

Zinc Omadine, (1-hydroxy-4-methyl-6-(2,4,4-trimethylpentyl)-2-pyridone and salts thereof and selenium sulphides are particularly preferred.

The antidandruff agent(s) ii) can be present in a content ranging from 0.001% to 10% by weight, preferably ranging from 0.1% to 5% by weight and more preferentially ranging from 0.2% to 2% by weight, with respect to the total weight of the composition of the invention.

Preferably, the product(s) (i)/antidandruff agent ii) weight ratio ranges from 0.1 to 50, more preferably still from 1 to 50, better still from 1 to 30.

For the purposes of the present invention, the term “fixing polymer” means any polymer that is capable, by application to the hair, of giving a shape to a head of hair or of holding the hair in an already acquired shape.

All the anionic, cationic, amphoteric and nonionic fixing polymers and mixtures thereof used in the art may be used in the compositions according to the present application.

The anionic fixing polymers generally used are polymers comprising groups derived from carboxylic acid, sulphonic acid or phosphoric acid and have a number-average molecular weight between around 500 and 5 000 000.

The carboxylic groups are provided by unsaturated carboxylic monoacid or diacid monomers such as those corresponding to the formula:

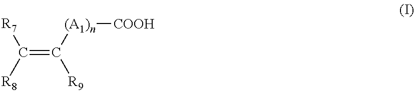

in which n is an integer from 0 to 10, A₁ denotes a methylene group optionally joined to the carbon atom of the unsaturated group or to the adjacent methylene group when n is greater than 1, via a heteroatom such as oxygen or sulphur, R₇ denotes a hydrogen atom or a phenyl or benzyl group, R₈ denotes a hydrogen atom, a lower alkyl or carboxyl group, R₉ denotes a hydrogen atom, a lower alkyl group, or a —CH₂—COOH, phenyl or benzyl group.

In the abovementioned formula, a lower alkyl group preferably denotes a group having 1 to 4 carbon atoms and in particular methyl and ethyl groups.

The anionic fixing polymers containing carboxylic groups that are preferred according to the invention are:

A) acrylic or methacrylic acid homopolymers or copolymers or salts thereof and in particular the products sold under the names Versicol® E or K by the company Allied Colloid and Ultrahold® by the company BASF, copolymers of acrylic acid and of acrylamide sold in the form of their sodium salts under the names Reten 421, 423 or 425 by the company Hercules, the sodium salts of polyhydroxycarboxylic acids;
B) copolymers of acrylic or methacrylic acid with a monoethylenic monomer such as ethylene, styrene, vinyl esters and acrylic or methacrylic acid esters, optionally grafted onto a polyalkylene glycol such as polyethylene glycol and optionally crosslinked. Such polymers are described in particular in French patent 1 222 944 and German patent application 2 330 956, the copolymers of this type comprising an optionally N-alkylated and/or hydroxyalkylated acrylamide unit in their chain as described in particular in Luxembourg patent applications 75370 and 75371 or sold under the name Quadramer by the company American Cyanamid. Mention may also be made of copolymers of acrylic acid and of C₁-C₄ alkyl methacrylate and terpolymers of vinylpyrrolidone, of acrylic acid and of methacrylate of C₁-C₂₀ alkyl, for example lauryl methacrylate, such as the product sold by the company ISP under the name Acrylidone LM and methacrylic acid/ethyl acrylate/tert-butyl acrylate terpolymers such as the product sold under the name Luvimer 100 P by the company BASF;

Mention may also be made of methacrylic acid/acrylic acid/ethyl acrylate/methyl methacrylate copolymers as an aqueous dispersion, sold under the name Amerhold® DR 25 by the company Amerchol;

C) crotonic acid copolymers, such as those comprising vinyl acetate or propionate units in their chain and optionally other monomers such as allyl esters or methallyl esters, vinyl ether or vinyl ester of a linear or branched saturated carboxylic acid with a long hydrocarbon-based chain, such as those containing at least 5 carbon atoms, it being possible for these polymers optionally to be grafted or crosslinked, or alternatively another vinyl, allyl or methallyl ester monomer of an α- or β-cyclic carboxylic acid. Such polymers are described, inter alia, in French patents 1 222 944, 1 580 545, 2 265 782, 2 265 781, 1 564 110 and 2 439 798. Commercial products that come under this category are the resins 28-29-30, 26-13-14 and 28-13-10 sold by the company National Starch;
D) copolymers of C₄-C₈ monounsaturated carboxylic acids or anhydrides selected from:
copolymers comprising (i) one or more maleic, fumaric or itaconic acids or anhydrides and (ii) at least one monomer selected from vinyl esters, vinyl ethers, vinyl halides, phenylvinyl derivatives, acrylic acid and its esters, the anhydride functions of these copolymers optionally being monoesterified or monoamidated. Such polymers are described, in particular, in U.S. Pat. Nos. 2,047,398, 2,723,248 and 2,102,113, and GB patent 839 805. Commercial products are especially those sold under the names Gantrez® AN or ES by the company ISP;
copolymers comprising (i) one or more maleic, citraconic or itaconic anhydride units and (ii) one or more monomers selected from allyl or methallyl esters optionally comprising one or more acrylamide, methacrylamide, α-olefin, acrylic or methacrylic ester, acrylic or methacrylic acid or vinylpyrrolidone groups in their chain,
   the anhydride functions of these copolymers optionally being monoesterified or monoamidated.

These polymers are described, for example, in French patents 2 350 384 and 2 357 241 by the Applicant;

E) polyacrylamides comprising carboxylate groups.

The homopolymers and copolymers comprising sulphonic groups are polymers comprising vinylsulphonic, styrenesulphonic, naphthalene-sulphonic or acrylamidoalkylsulphonic units.

These polymers may especially be selected from:
polyvinylsulphonic acid salts having a molecular weight of approximately between 1000 and 100 000, and also the copolymers with an unsaturated comonomer such as acrylic or methacrylic acids and their esters, and also acrylamide or its derivatives, vinyl ethers and vinylpyrrolidone;
polystyrenesulphonic acid salts such as the sodium salts that are sold for example under the names Flexae 500 and Flexae 130 by National Starch. These compounds are described in patent FR 2 198 719;
polyacrylamidesulphonic acid salts, such as those mentioned in U.S. Pat. No. 4,128,631 and more particularly polyacrylamidoethylpropanesulphonic acid sold under the name Cosmedia Polymer HSP 1180 by Henkel.

As another anionic fixing polymer that can be used according to the invention, mention may be made of the branched block anionic polymer sold under the name Fixate G-100 by the company Noveon.

According to the invention, the anionic fixing polymers are preferably selected from acrylic acid copolymers such as the acrylic acid/ethyl acrylate/N-tert-butylacrylamide terpolymers sold in particular under the name Ultrahold® Strong by the company BASF, copolymers derived from crotonic acid, such as vinyl acetate/vinyl tert-butylbenzoate/crotonic acid terpolymers and the crotonic acid/vinyl acetate/vinyl neododecanoate terpolymers sold especially under the name Resin 28-29-30 by the company National Starch, polymers derived from maleic, fumaric or itaconic acids or anhydrides with vinyl esters, vinyl ethers, vinyl halides, phenylvinyl derivatives and acrylic acid and esters thereof, such as the methyl vinyl ether/monoesterified maleic anhydride copolymers sold, for example, under the name Gantrez® by the company ISP, the copolymers of methacrylic acid and of methyl methacrylate sold under the name Eudragit® L by the company Rohm Pharma, the copolymers of methacrylic acid and of ethyl acrylate sold under the name Luvimer® MAEX or MAE by the company BASF, the vinyl acetate/crotonic acid copolymers sold under the name Luviset CA 66 by the company BASF, the vinyl acetate/crotonic acid copolymers grafted with polyethylene glycol sold under the name Aristoflex® A by the company BASF, and the polymer sold under the name Fixate G-100 by the company Noveon.

Among the anionic fixing polymers mentioned above, it is more particularly preferred in the context of the present invention to use the methyl vinyl ether/monoesterified maleic anhydride copolymers sold under the name Gantrez® ES 425 by the company ISP, the acrylic acid/ethyl acrylate/N-tert-butylacrylamide terpolymers sold under the name Ultrahold® Strong by the company BASF, the copolymers of methacrylic acid and of methyl methacrylate sold under the name Eudragit® L by the company Rohm Pharma, the vinyl acetate/vinyl tert-butylbenzoate/crotonic acid terpolymers and the crotonic acid/vinyl acetate/vinyl neododecanoate terpolymers sold under the name Resin 28-29-30 by the company National Starch, the copolymers of methacrylic acid and of ethyl acrylate sold under the name Luvimer® MAEX or MAE by the company BASF, the vinylpyrrolidone/acrylic acid/lauryl methacrylate terpolymers sold under the name Acrylidone® LM by the company ISP and the polymer sold under the name Fixate G-100 by the company Noveon.

The cationic fixing film-forming polymers that can be used according to the present invention are preferably selected from polymers comprising primary, secondary, tertiary and/or quaternary amine groups forming part of the polymer chain or directly attached thereto, and having a molecular weight of between 500 and about 5 000 000 and preferably between 1000 and 3 000 000.

Among these polymers, mention may be made more particularly of the following cationic polymers:
(1) homopolymers or copolymers derived from acrylic or methacrylic esters or amides and comprising at least one of the units of the following formulae:

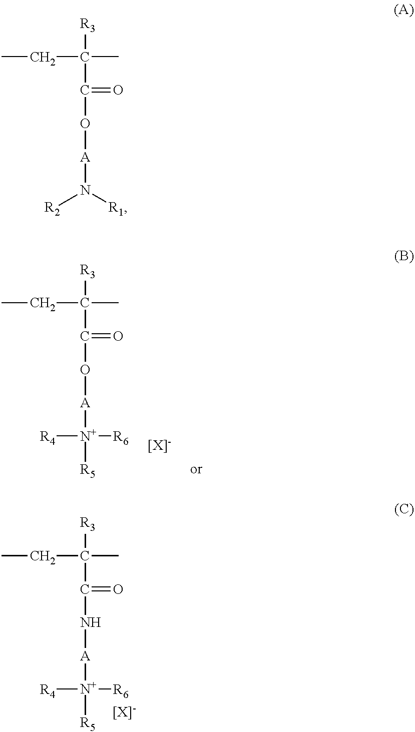

in which:
R₃ denotes a hydrogen atom or a CH₃ radical;
A is a linear or branched alkyl group comprising 1 to 6 carbon atoms or a hydroxyalkyl group comprising 1 to 4 carbon atoms;
R₄, R₅ and R₆, which may be identical or different, represent an alkyl group having from 1 to 18 carbon atoms or a benzyl radical;
R₁ and R₂, which may be identical or different, each represent a hydrogen atom or an alkyl group having from 1 to 6 carbon atoms;
X denotes a methosulphate anion or a halide such as chloride or bromide.

The copolymers of the family (1) also contain one or more units derived from comonomers that may be selected from the family of acrylamides, methacrylamides, diacetone acrylamides, acrylamides and methacrylamides substituted on the nitrogen with lower (C₁-C₄) alkyl groups, groups derived from acrylic or methacrylic acids or esters thereof, vinyllactams such as vinylpyrrolidone or vinylcaprolactam, and vinyl esters.

Thus, among these copolymers of the family (1), mention may be made of:
copolymers of acrylamide and of dimethylaminoethyl methacrylate quaternized with dimethyl sulphate or with a dimethyl halide, such as that sold under the name Hercofloc® by the company Hercules,
copolymers of acrylamide and of methacryloyloxyethyltrimethylammonium chloride, described, for example, in Patent Application EP-A-080 976 and sold under the name Bina Quat P 100 by the company Ciba Geigy,
the copolymer of acrylamide and of methacryloyloxyethyltrimethylammonium methosulphate such as that sold under the name Reten by the company Hercules,
quaternized or non-quaternized vinylpyrrolidone/dialkylaminoalkyl acrylate or methacrylate copolymers, such as the products sold under the name Gafquat® by the company ISP, such as, for example, Gafquat® 734 or Gafquat® 755, or alternatively the products known as Copolymer® 845, 958 and 937. These polymers are described in detail in French Patents 2 077 143 and 2 393 573,
fatty-chain polymers containing a vinylpyrrolidone unit, such as the products sold under the name Styleze W20 and Styleze W10 by the company ISP,
dimethylaminoethyl methacrylate/vinylcaprolactam/vinylpyrrolidone terpolymers, such as the product sold under the name Gaffix VC 713 by the company ISP, and
quaternized vinylpyrrolidone/dimethylaminopropylmethacrylamide copolymers, such as the products sold under the name Gafquat® HS 100 by the company ISP;
(2) cationic polysaccharides, preferably containing quaternary ammonium, such as those described in U.S. Pat. Nos. 3,589,578 and 4,031,307, such as guar gums containing trialkylammonium cationic groups; Such products are sold in particular under the trade names Jaguar C13S, Jaguar C 15 and Jaguar C 17 by the company Meyhall;
(3) quaternary copolymers of vinylpyrrolidone and of vinylimidazole;
(4) chitosans or salts thereof; the salts that can be used are, in particular, chitosan acetate, lactate, glutamate, gluconate or pyrrolidonecarboxylate. Among these compounds, mention may be made of chitosan having a degree of deacetylation of 90.5% by weight, sold under the name Kytan Brut Standard by the company Aber Technologies, and chitosan pyrrolidonecarboxylate sold under the name Kytamer® PC by the company Amerchol;
(5) cationic cellulose derivatives such as copolymers of cellulose or of cellulose derivatives grafted with a water-soluble monomer comprising a quaternary ammonium, and disclosed in particular in U.S. Pat. No. 4,131,576, such as hydroxyalkylcelluloses, for instance hydroxymethyl-, hydroxyethyl- or hydroxypropylcelluloses grafted in particular with a methacryloyloxyethyltrimethylammonium, methacrylamidopropyltrimethylammonium or dimethyldiallylammonium salt.

The products sold corresponding to this definition are, more particularly, the products sold under the name Celquat L 200 and Celquat H 100 by the company National Starch.

The amphoteric fixing polymers that can be used in accordance with the invention can be selected from polymers comprising units B and C distributed randomly in the polymer chain, where B denotes a unit derived from a monomer comprising at least one basic nitrogen atom and C denotes a unit derived from an acid monomer comprising one or more carboxylic or sulphonic groups, or alternatively B and C can denote groups derived from carboxybetaine or sulphobetaine zwitterionic monomers;

B and C can also denote a cationic polymer chain comprising primary, secondary, tertiary or quaternary amine groups, in which at least one of the amine groups bears a carboxylic or sulphonic group connected via a hydrocarbon group or alternatively B and C form part of a chain of a polymer containing an α,β-dicarboxylic ethylene unit in which one of the carboxylic groups has been made to react with a polyamine comprising one or more primary or secondary amine groups.

The amphoteric fixing polymers corresponding to the definition given above that are more particularly preferred are selected from the following polymers:

(1) copolymers having acidic vinyl units and basic vinyl units, such as those resulting from the copolymerization of a monomer derived from a vinyl compound bearing a carboxylic group such as, more particularly, acrylic acid, methacrylic acid, maleic acid, α-chloroacrylic acid, and a basic monomer derived from a substituted vinyl compound containing at least one basic atom, such as, more particularly, dialkylaminoalkyl methacrylate and acrylate, dialkylaminoalkylmethacrylamides and acrylamides. Such compounds are described in U.S. Pat. No. 3,836,537.
(2) Polymers comprising units derived from:
a) at least one monomer selected from acrylamides and methacrylamides substituted on the nitrogen atom by an alkyl group,
b) at least one acidic comonomer containing one or more reactive carboxylic groups, and
c) at least one basic comonomer such as esters containing primary, secondary, tertiary and quaternary amine substituents of acrylic and methacrylic acids and the product of quaternization of dimethylaminoethyl methacrylate with dimethyl or diethyl sulphate.

The N-substituted acrylamides or methacrylamides that are more particularly preferred according to the invention are compounds in which the alkyl groups contain from 2 to 12 carbon atoms and more particularly N-ethylacrylamide, N-tert-butylacrylamide, N-tert-octylacrylamide, N-octylacrylamide, N-decylacrylamide, N-dodecylacrylamide and the corresponding methacrylamides.

The acidic comonomers are selected more particularly from acrylic acid, methacrylic acid, crotonic acid, itaconic acid, maleic acid and fumaric acid and alkyl monoesters, having 1 to 4 carbon atoms, of maleic or fumaric acids or anhydrides.

The preferred basic comonomers are aminoethyl, butylaminoethyl, N,N′-dimethylaminoethyl and N-tert-butylaminoethyl methacrylates.

Use is made particularly of the copolymers whose CTFA (4th edition, 1991) name is octylacrylamide/acrylates/butylaminoethyl methacrylate copolymer, such as the products sold under the name Amphomer® or Lovocryl® 47 by the company National Starch.
(3) Crosslinked and acylated polyaminoamides partially or totally deriving from polyaminoamides of general formula:

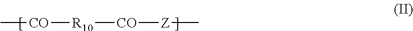

in which R₁₀ represents a divalent group derived from a saturated dicarboxylic acid, a mono- or dicarboxylic aliphatic acid containing an ethylenic double bond, an ester of a lower alkanol, having 1 to 6 carbon atoms, of these acids, or a group derived from the addition of any one of said acids to a bis(primary) or bis(secondary) amine, and Z denotes a group derived from a bis(primary), mono- or bis(secondary) polyalkylene-polyamine and preferably represents:
a) in proportions of from 60 to 100 mol %, the group:

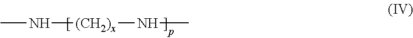

where x=2 and p=2 or 3, or alternatively x=3 and p=2 this group being derived from diethylenetriamine, from triethylenetetramine or from dipropylenetriamine;
b) in proportions of from 0 to 40 mol %, the group (IV) above in which x=2 and p=1 and which is derived from ethylenediamine, or the group derived from piperazine:

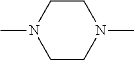

c) in proportions of from 0 to 20 mol %, the —NH—(CH₂)₆—NH— group being derived from hexamethylenediamine,
   these polyaminoamides being crosslinked by addition reaction of a difunctional crosslinking agent selected from epihalohydrins, diepoxides, dianhydrides and bis-unsaturated derivatives, using from 0.025 to 0.35 mol of crosslinking agent per amine group of the polyaminoamide and acylated by the action of acrylic acid, chloroacetic acid or an alkane sultone, or salts thereof.

The saturated carboxylic acids are preferably selected from acids having 6 to 10 carbon atoms, such as adipic acid, 2,2,4-trimethyladipic acid and 2,4,4-trimethyladipic acid, terephthalic acid, acids containing an ethylenic double bond such as, for example, acrylic acid, methacrylic acid and itaconic acid.

The alkane sultones used in the acylation are preferably propane sultone or butane sultone; the salts of the acylating agents are preferably the sodium or potassium salts.

(4) Polymers comprising zwitterionic units of formula:

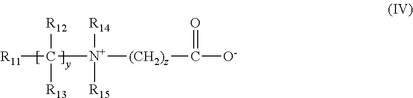

in which R₁₁ denotes a polymerizable unsaturated group such as an acrylate, methacrylate, acrylamide or methacrylamide group, y and z represent an integer from 1 to 3, R₁₂ and R₁₃ represent a hydrogen atom, a methyl, ethyl or propyl group, R₁₄ and R₁₅ represent a hydrogen atom or an alkyl group such that the sum of the carbon atoms in R₁₄ and R₁₅ does not exceed 10.

The polymers comprising such units can also comprise units derived from non-zwitterionic monomers such as dimethyl- or diethylaminoethyl acrylate or methacrylate or alkyl acrylates or methacrylates, acrylamides or methacrylamides or vinyl acetate.

By way of example, mention may be made of the copolymers of methyl methacrylate/methyl dimethylcarboxymethylammonioethyl methacrylate such as the product sold under the name Diaformer Z301 by the company Sandoz.
(5) Polymers derived from chitosan comprising monomer units corresponding to the following formulae:

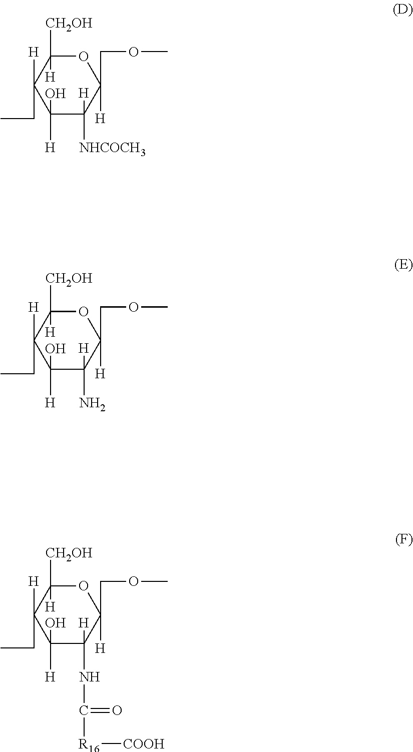

the unit (D) being present in proportions of between 0 and 30%, the unit (E) in proportions of between 5% and 50% and the unit (F) in proportions of between 30% and 90%, it being understood that, in this unit (F), R₁₆ represents a group of formula:

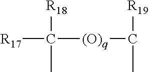

in which, if q=0, R₁₇, R₁₈ and R₁₉, which are identical or different, each represent a hydrogen atom, a methyl, hydroxyl, acetoxy or amino residue, a monoalkylamine residue or a dialkylamine residue that are optionally interrupted by one or more nitrogen atoms and/or optionally substituted by one or more amine, hydroxyl, carboxyl, alkylthio or sulphonic groups, an alkylthio residue in which the alkyl group bears an amino residue, at least one of the groups R₁₇, R₁₈ and R₁₉ being, in this case, a hydrogen atom;
or, if q=1, R₁₇, R₁₈ and R₁₉ each represent a hydrogen atom, as well as the salts formed by these compounds with bases or acids.
(6) Polymers corresponding to the general formula (V) are described, for example, in French patent 1 400 366:

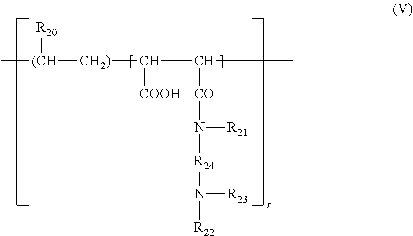

in which R₂₀ represents a hydrogen atom, a CH₃O, CH₃CH₂O or phenyl group, R₂₁ denotes a hydrogen atom or a lower alkyl group such as methyl or ethyl, R₂₂ denotes a hydrogen atom or a C₁-C₆ lower alkyl group such as methyl or ethyl, R₂₃ denotes a C₁-C₆ lower alkyl group such as methyl or ethyl or a group corresponding to the formula: —R₂₄—N(R₂₂)₂, R₂₄ representing a —CH₂—CH₂—, —CH₂—CH₂—CH₂— or —CH₂—CH(CH₃)— group, and R₂₂ having the above-mentioned meanings.
(7) Polymers derived from the N-carboxyalkylation of chitosan, such as N-carboxymethylchitosan or N-carboxybutylchitosan sold under the name “Evalsan” by the company Jan Dekker.
(8) Amphoteric polymers of the type -D-X-D-X selected from:
a) Polymers obtained by the action of chloroacetic acid or sodium chloroacetate on compounds comprising at least one unit of formula:

-D-X-D-X-D- (VI)

where D denotes a group

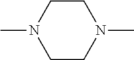

and X denotes the symbol E or E′, E or E′, which may be identical or different, denote a divalent group that is an alkylene group with a straight or branched chain containing up to 7 carbon atoms in the main chain, which is unsubstituted or substituted by hydroxyl groups and which can comprise, in addition to the oxygen, nitrogen and sulphur atoms, 1 to 3 aromatic and/or heterocyclic rings; the oxygen, nitrogen and sulphur atoms being present in the form of ether, thioether, sulphoxide, sulphone, sulphonium, alkylamine or alkenylamine groups, hydroxyl, benzylamine, amine oxide, quaternary ammonium, amide, imide, alcohol, ester and/or urethane groups.
b) Polymers of formula:

-D-X-D-X- (VI′)

where D denotes a group

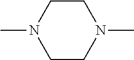

and X denotes the symbol E or E′ and at least once E′; E having the meaning given above and E′ is a divalent group that is an alkylene group with a straight or branched chain having up to 7 carbon atoms in the main chain, which is unsubstituted or substituted with one or more hydroxyl groups and containing one or more nitrogen atoms, the nitrogen atom being substituted with an alkyl chain that is optionally interrupted by an oxygen atom and necessarily comprising one or more carboxyl functions or one or more hydroxyl functions and betainized by reaction with chloroacetic acid or sodium chloroacetate.
(9) (C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkylaminoalkanol. These copolymers can also comprise other vinyl comonomers such as vinylcaprolactam.

Among the amphoteric fixing polymers mentioned above, the ones that are most particularly preferred according to the invention are those of family (3), such as the copolymers whose CTFA name is octylacrylamide/acrylates/butylaminoethyl methacrylate copolymer, such as the products sold under the names Amphomer®, Amphomer® LV 71 or Lovocryl® 47 by the company National Starch and those of family (4) such as the methyl methacrylate/methyl dimethylcarboxymethylammonioethyl methacrylate copolymers, sold, for example, under the name Diaformer Z301 by the company Sandoz.

The nonionic fixing polymers that may be used according to the present invention are selected, for example, from:
polyalkyloxazolines;
vinyl acetate homopolymers;
vinyl acetate copolymers, for instance copolymers of vinyl acetate and of acrylic ester; copolymers of vinyl acetate and of ethylene, or copolymers of vinyl acetate and of maleic ester, for example of dibutyl maleate;
homopolymers and copolymers of acrylic esters, for instance copolymers of alkyl acrylates and of alkyl methacrylates, such as the products sold by the company Rohm & Haas under the names Primal® AC-261 K and Eudragie NE 30 D, by the company BASF under the name 8845, or by the company Hoechst under the name Appretae N9212;
acrylonitrile copolymers and copolymers of a nonionic monomer selected, for example, from butadiene and alkyl (meth)acrylates; mention may be made of the products sold under the name CJ 0601 B by the company Rohm & Haas;
styrene homopolymers;
styrene copolymers, for instance copolymers of styrene and of alkyl (meth)acrylate, such as the products Mowilite LDM 6911, Mowilite DM 611 and Mowilite LDM 6070 sold by the company Hoechst, and the products Rhodopas® SD 215 and Rhodopas® DS 910 sold by the company Rhône-Poulenc; copolymers of styrene, of alkyl methacrylate and of alkyl acrylate; copolymers of styrene and of butadiene; or copolymers of styrene, of butadiene and of vinylpyridine;
polyamides;
vinyllactam homopolymers such as vinylpyrrolidone homopolymers and such as the polyvinylcaprolactam sold under the name Luviskol® Plus by the company BASF; and
vinyllactam copolymers such as a poly(vinylpyrrolidone/vinyllactam) copolymer sold under the trade name Luvitec® VPC 55K65W by the company BASF, poly(vinylpyrrolidone/vinyl acetate) copolymers, such as those sold under the name PVPVA® S630L by the company ISP, Luviskol® VA 73, VA 64, VA 55, VA 37 and VA 28 by the company BASF; and poly(vinylpyrrolidone/vinyl acetate/vinyl propionate) terpolymers, for instance the product sold under the name Luviskol® VAP 343 by the company BASF.

The alkyl groups of the nonionic polymers mentioned above preferably have from 1 to 6 carbon atoms.

According to the invention, it is also possible to use fixing polymers of grafted silicone type comprising a polysiloxane portion and a portion consisting of a non-silicone organic chain, one of the two portions constituting the main chain of the polymer, and the other being grafted onto said main chain.

These polymers are described, for example, in patent applications EP-A-0 412 704, EP-A-0 412 707, EP-A-0 640 105 and WO 95/00578, EP-A-0 582 152 and WO 93/23009 and U.S. Pat. No. 4,693,935, U.S. Pat. No. 4,728,571 and U.S. Pat. No. 4,972,037.

These polymers may be amphoteric, anionic or nonionic, and are preferably anionic or nonionic.

Such polymers are, for example, copolymers that can be obtained by free radical polymerization from the monomer mixture formed from:

a) 50 to 90% by weight of tert-butyl acrylate;
b) 0 to 40% by weight of acrylic acid;
c) 5 to 40% by weight of a silicone macromer of formula:

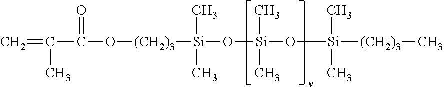

in which v is a number ranging from 5 to 700, the weight percentages being calculated relative to the total weight of the monomers.

Other examples of grafted silicone polymers are, in particular, polydimethylsiloxanes (PDMSs) onto which are grafted, via a thiopropylene-type connecting chain, mixed polymer units of the poly(meth)acrylic acid type and of the polyalkyl (meth)acrylate type and polydimethylsiloxanes (PDMSs) onto which are grafted, via a thiopropylene-type connecting chain, polymer units of the polyisobutyl (meth)acrylate type.

Another type of silicone fixing polymer that may be mentioned is the product Luviflex® Silk sold by the company BASF.

As fixing polymers it is also possible to use functionalized or non-functionalized, cationic, nonionic, anionic or amphoteric, silicone or non-silicone polyurethanes, or mixtures thereof.

The polyurethanes particularly intended by the present invention are those disclosed in patent applications EP 0 751 162, EP 0 637 600, EP 0 648 485 and FR 2 743 297, of which the Applicant is the Proprietor, and patent applications EP 0 656 021 and WO 94/03510 from the company BASF and EP 0 619 111 from the company National Starch.

As polyurethanes that are particularly suitable for the present invention, mention may be made of the products sold under the names Luviset Pur® and Luviset® Si Pur by the company BASF.

In one preferred variant, the fixing polymers are nonionic or anionic.

In another preferred variant, the fixing polymers are soluble in the composition of the invention.

The concentration of fixing polymer(s) used in the compositions according to the present invention may range from 0.1 to 20%, preferably from 0.5 to 10% by weight relative to the total weight of the composition.

The composition according to the invention may comprise one or more solvents having a dielectric constant of greater than 15 at 20° C.

Such solvents are considered to be polar solvents.

Preferably, the solvent or solvents are selected from isopropanol, ethanol, acetone, water or mixtures thereof.

More preferably still, the solvent or solvents are selected from solvents having a dielectric constant of greater than 25 at 20° C. The preferred solvents are water, ethanol or mixtures thereof.

The concentration of solvent(s) having a dielectric constant of greater than 15 at 20° C. used in the compositions according to the present invention may range from 0.1 to 99.8% by weight, preferably from 15 to 99% by weight, better still from 50 to 95% by weight relative to the total weight of the composition.

The composition according to the invention may comprise one or more reducing agents. Preferably, the reducing agent or agents are selected from sulphites and reducing agents of formula (I):

(H)_{q}X(R)ᵣ (I)

in which X represents P, S or SO₂, q is equal to 0 or 1, r is equal to 1 or 2 or 3 and R is a linear or branched, saturated or unsaturated C₁-C₂₀ hydrocarbon-based radical optionally interrupted by a heteroatom, and optionally comprising substituents selected from hydroxyl, halogen, amino, carboxyl, ((C₁-C₃₀)alkoxy)carbonyl, amido, ((C₁-C₃₀)alkyl)aminocarbonyl and ((C₁-C₃₀)acyl)amino groups, a mono- or di((C₁-C₃₀)alkyl)amino group or a mono- or dihydroxy((C₁-C₃₀)alkyl)amino group,
or a salt thereof in combination with a base.

In particular, mention may be made of sodium or ammonium sulphites, thioglycolic acid or thiolactic acid and their ester and amide derivatives, especially glycerol monothioglycolate, cysteamine and its C₁-C₄ acyl derivatives such as N-acetylcysteamine or N-propionylcysteamine, cysteine, N-acetylcysteine, thiomalic acid, panthetein, 2,3-dimercaptosuccinic acid, N-(mercaptoalkyl)-ω-hydroxyalkylamides such as those described in patent application EP-A-354 835, N-mono- or N,N-dialkylmercapto-4-butyramides such as those described in patent application EP-A-368 763, aminomercaptoalkylamides such as those described in patent application EP-A-432 000, derivatives of N-(mercaptoalkyl)succinamic acids and N-(mercaptoalkyl)succinimides such as those described in patent application EP-A-465 342, alkylaminomercaptoalkylamides such as those described in patent application EP-A-514 282, the azeotropic mixture of 2-hydroxypropyl thioglycolate and (2-hydroxy-1-methyl)ethyl thioglycolate described in patent application FR-A-2 679 448, mercaptoalkylaminoamides such as those described in patent application FR-A-2 692 481, the N-mercaptoalkylalkanediamides described in patent application EP-A-653 202, and the derivatives of formamidine sulphinic acid. Where appropriate, these compounds may be used in the form of salts.

Generally, the reducing agent or agents represent from 0.05 to 30%, preferably from 1 to 20%, by weight of the total weight of the composition.

The pH of the composition according to the invention is preferably between 4 and 11, and more particularly between 6 and 10.

The pH is generally obtained using an alkaline agent such as, for example, aqueous ammonia, monoethanolamine, diethanolamine, triethanolamine, 1,3-propanediamine, an alkali metal or ammonium carbonate or bicarbonate, an organic carbonate such as guanidine carbonate, an alkali metal hydroxide, or using an acidifying agent such as, for example, hydrochloric acid, acetic acid, lactic acid, oxalic acid or boric acid, or else using standard buffers such as, for example, borate buffers, phosphate buffers or TRIS (based on tris(hydroxymethyl)aminomethane) buffers.

The composition according to the invention may comprise water, one or more organic solvents or a mixture of water and one or more organic solvents, the organic solvents preferably being selected from C₁-C₄ lower alcohols such as ethanol, isopropanol, tert-butanol or n-butanol; polyols such as glycerol, propylene glycol and polyethylene glycols; and mixtures thereof.

In the composition according to the invention, the water may be present in a content ranging from 10% to 95% by weight and preferably ranging from 20% to 95% by weight relative to the total weight of the composition.

The composition according to the invention may also comprise one or more additional thickeners that are different from the acrylic thickening polymers as described above.

The additional thickener(s) may be selected from sodium chloride, fatty acid amides obtained from a C₁₀-C₃₀ carboxylic acid (coconut acid monoisopropanolamide, diethanolamide or monoethanolamide, oxyethylenated alkyl ether carboxylic acid monoethanolamide), nonionic cellulosic thickeners (hydroxyethyl cellulose, hydroxypropyl cellulose, carboxymethyl cellulose), guar gum and nonionic derivatives thereof (hydroxypropyl guar), gums of microbial origin (xanthan gum, scleroglucan gum) and associative polymers that are different from the acrylic thickening polymers.

The associative polymer(s) that can be used according to the invention are water-soluble polymers which in an aqueous medium are capable of reversible association with one another or with other molecules.

Their chemical structure comprises hydrophilic zones, and hydrophobic zones characterized by at least one fatty chain containing preferably from 10 to 30 carbon atoms.

The associative polymer(s) that may be used according to the invention and that are different from the acrylic thickening polymers may be of anionic, cationic, amphoteric or nonionic type, such as the polymers sold under the names Elfacos T210 or T212 by the company Akzo.

Among all the additional thickeners mentioned, the thickener(s) is (are) preferably selected from fatty acid amides obtained from a C₁₀-C₃₀ carboxylic acid.

When they are present, the cosmetic composition comprises preferably from 0.1% to 20% by weight, and better still from 0.2% to 10% by weight, of additional thickener(s), relative to the total weight of the composition.

The composition according to the invention may also comprise one or more conditioning agents.

According to the present invention, the term “conditioning agent” denotes any compound that can improve the cosmetic properties of the hair, in particular the softness, disentangling, feel and static electricity.

The conditioning agent is preferably selected from the group comprising cationic polymers, cationic surfactants, silicones such as organosiloxanes, linear or branched C₈-C₃₀ hydrocarbons, linear or branched C₈-C₃₀ fatty alcohols, esters of C₈-C₃₀ fatty acid and C₁-C₃₀ alcohol, and especially esters of C₈-C₃₀ fatty acid and C₈-C₃₀ fatty alcohol, esters of C₁-C₇, acid or diacid and C₈-C₃₀ fatty alcohol, ceramides or ceramide analogues, and mixtures of these compounds.

The term “cationic polymer” means a polymer that is positively charged when it is contained in the composition according to the invention. This polymer may bear one or more positive permanent charges or may contain one or more cationizable functions in the composition according to the invention.

The cationic polymer(s) that may be used as conditioning agents according to the present invention are preferably selected from polymers comprising primary, secondary, tertiary and/or quaternary amine groups forming part of the polymer chain or directly attached thereto, and having a molecular weight of between 500 and about 5 000 000 and preferably between 1000 and 3 000 000.

Among the cationic polymers that may be mentioned more particularly are polymers of the polyamine, polyaminoamide and polyquaternary ammonium type. These are known products. They are described, for example, in French Patents 2 505 348 and 2 542 997.

Among these polymers, mention may be made of:

(1) homopolymers or copolymers derived from acrylic or methacrylic esters or amides and comprising at least one of the units of the following formulae:

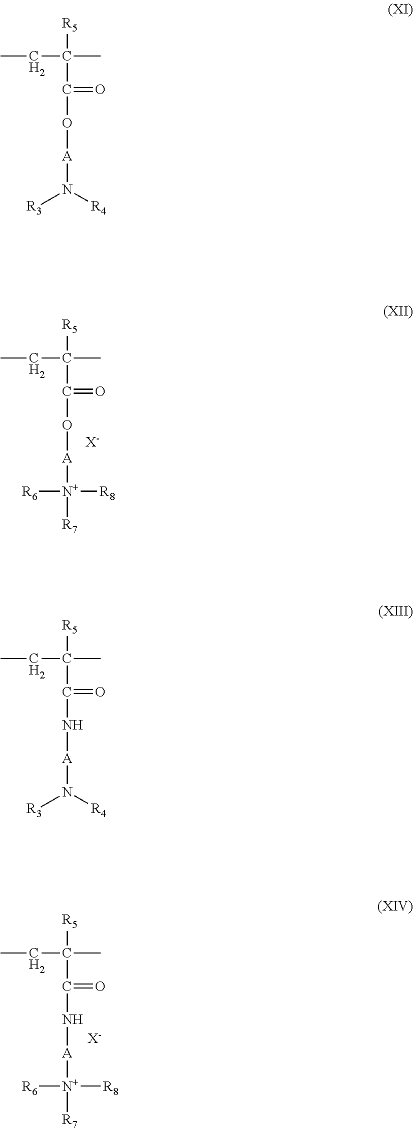

in which:
R₃ and R₄, which are identical or different, represent a hydrogen atom or an alkyl group having from 1 to 6 carbon atoms, and preferably methyl or ethyl;
R₅, which are identical or different, denote a hydrogen atom or a CH₃ group; A, which are identical or different, represent a linear or branched alkyl group having from 1 to 6 carbon atoms, preferably 2 or 3 carbon atoms, or a hydroxyalkyl group having from 1 to 4 carbon atoms;
R₆, R₇, R₈, which are identical or different, represent an alkyl group having from 1 to 18 carbon atoms or a benzyl group, and preferably an alkyl group having from 1 to 6 carbon atoms;
X⁻ denotes an anion derived from an organic or inorganic acid, such as a methosulphate anion, or a halide such as chloride or bromide.

The copolymers of the family (1) may further contain one or more units deriving from comonomers which may be selected from the family of acrylamides, methacrylamides, diacetone acrylamides, acrylamides and methacrylamides substituted on the nitrogen by lower alkyls (C₁-C₄), acrylic acids or methacrylic acids or esters thereof, vinyllactams such as vinylpyrrolidone or vinylcaprolactam, and vinyl esters.

Thus, among these copolymers of the family (1), mention may be made of:
copolymers of acrylamide and of dimethylaminoethyl methacrylate quaternized with dimethyl sulphate or with a dimethyl halide, such as that sold under the name Hercofloc by the company Hercules,
copolymers of acrylamide and of methacryloyloxyethyltrimethylammonium chloride, described, for example, in Patent Application EP-A-080 976 and sold under the name Bina Quat P 100 by the company Ciba Geigy,
the copolymer of acrylamide and of methacryloyloxyethyltrimethylammonium methosulphate sold under the name Reten by the company Hercules,
quaternized or non-quaternized vinylpyrrolidone/dialkylaminoalkyl acrylate or methacrylate copolymers, such as the products sold under the name Gafquat by the company ISP, such as, for example, Gafquat 734 or Gafquat 755, or alternatively the products known as Copolymer 845, 958 and 937. These polymers are described in detail in French Patents 2 077 143 and 2 393 573,
dimethylaminoethyl methacrylate/vinylcaprolactam/vinylpyrrolidone terpolymers, such as the product sold under the name Gaffix VC 713 by the company ISP,
vinylpyrrolidone/methacrylamidopropyldimethylamine copolymers sold in particular under the name Styleze CC 10 by the company ISP,
quaternized vinylpyrrolidone/dimethylaminopropyl methacrylamide copolymers, such as the product sold under the name Gafquat HS 100 by the company ISP, and
the crosslinked polymers of methacryloyloxy(C₁-C₄)alkyl tri(C₁-C₄)alkylammonium salts, such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with an olefinically unsaturated compound, more particularly methylenebisacrylamide. A crosslinked acrylamide/methacryloyloxyethyltrimethyl-ammonium chloride copolymer (20/80 by weight) in the form of a dispersion containing 50% by weight of said copolymer in mineral oil can be used more particularly. This dispersion is sold under the name Salcare® SC 92 by the company Ciba. A crosslinked methacryloyloxyethyltrimethylammonium chloride homopolymer, for example as a dispersion in mineral oil or in a liquid ester, can also be used. These dispersions are sold under the names Salcare® SC 95 and Salcare® SC 96 by the company Ciba.
   (2) Polymers composed of piperazinyl units and of divalent alkylene or hydroxyalkylene groups containing straight or branched chains, optionally interrupted by oxygen, sulphur or nitrogen atoms or by aromatic or heterocyclic rings, and also the oxidation and/or quaternization products of these polymers. Such polymers are especially described in French Patents 2 162 025 and 2 280 361.
   (3) Water-soluble polyaminoamides prepared in particular by polycondensation of an acidic compound with a polyamine; these polyaminoamides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyaminoamide; these polyaminoamides can be alkylated or, if they comprise one or more tertiary amine functions, they can be quaternized. Such polymers are especially described in French Patents 2 252 840 and 2 368 508.
   (4) Polyaminoamide derivatives resulting from the condensation of polyalkylene polyamines with polycarboxylic acids followed by alkylation with difunctional agents. Mention may be made, for example, of adipic acid/dialkylamino-hydroxyalkyldialkylenetriamine polymers in which the alkyl groups contain from 1 to 4 carbon atoms and preferably denote a methyl, ethyl or propyl group, and the alkylene groups contain from 1 to 4 carbon atoms and preferably denote the ethylene group. Such polymers are especially described in French Patent 1 583 363.

Among these derivatives, mention may be made more particularly of the adipic acid/dimethylaminohydroxypropyl/diethylenetriamine polymers sold under the name Cartaretine F, F4 or F8 by the company Sandoz.

(5) The polymers obtained by reaction of a polyalkylene polyamine containing two primary amine groups and at least one secondary amine group with a dicarboxylic acid selected from diglycolic acid and saturated aliphatic dicarboxylic acids having from 3 to 8 carbon atoms. The molar ratio between the polyalkylene polyamine and the dicarboxylic acid is between 0.8:1 and 1.4:1; the polyaminoamide resulting therefrom is reacted with epichlorohydrin in a molar ratio of epichlorohydrin relative to the secondary amine group of the polyaminoamide of between 0.5:1 and 1.8:1. Such polymers are described in particular in U.S. Pat. Nos. 3,227,615 and 2,961,347. Polymers of this type are sold in particular under the name Hercosett 57 by the company Hercules Inc. or alternatively under the name PD 170 or Delsette 101 by the company Hercules in the case of the adipic acid/epoxypropyl/diethylenetriamine copolymer.
(6) Cyclopolymers of alkyldiallylamine or of dialkyldiallylammonium, such as the homopolymers or copolymers containing, as main constituent of the chain, units corresponding to formula (XV) or (XVI):

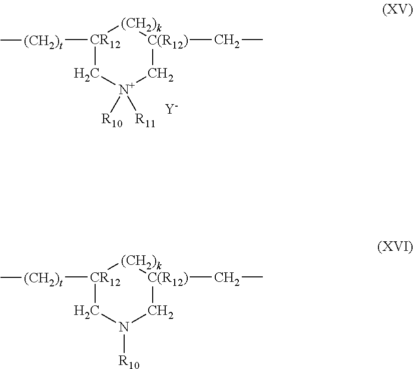

in which formulae: k and t are equal to 0 or 1, the sum k+t being equal to 1; R₁₂ denotes a hydrogen atom or a methyl group; R₁₀ and R₁₁, independently of one another, denote an alkyl group having from 1 to 6 carbon atoms, a hydroxyalkyl group in which the alkyl group has preferably 1 to 5 carbon atoms, a lower (C₁-C₄) amidoalkyl group, or else R₁₀ and R₁₁ may, together with the nitrogen atom to which they are attached, denote heterocyclic groups, such as piperidyl or morpholinyl; Y⁻ is an anion such as bromide, chloride, acetate, borate, citrate, tartrate, bisulphate, bisulphite, sulphate or phosphate. These polymers are especially described in French Patent 2 080 759 and in its Certificate of Addition 2 190 406.
R₁₀ and R₁₁, independently of one another, denote preferably an alkyl group having from 1 to 4 carbon atoms.

Among the polymers defined above, mention may be made more particularly of the dimethyldiallylammonium chloride homopolymer sold under the name Merquat 100 by the company Nalco (and its homologues of low weight-average molecular weights) and the copolymers of diallyldimethylammonium chloride and of acrylamide, sold under the name Merquat 550.

(7) The quaternary diammonium polymer containing repeating units corresponding to the formula (XVII):

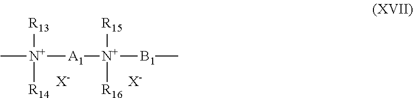

in which formula (XVII):
R₁₃, R₁₄, R₁₅ and R₁₆, which may be identical or different, represent aliphatic, alicyclic or arylaliphatic groups containing from 1 to 20 carbon atoms or lower (C₁-C₄)hydroxyalkylaliphatic groups, or alternatively R₁₃, R₁₄, R₁₅ and R₁₆, together or separately, constitute, with the nitrogen atoms to which they are attached, heterocycles optionally containing a second heteroatom other than nitrogen, or alternatively R₁₃, R₁₄, R₁₅ and R₁₆ represent a linear or branched C₁-C₆ alkyl group substituted with a nitrile, ester, acyl or amide group or a —CO—O—R₁₇-D or —CO—NH—R₁₇-D group where R₁₇ is an alkylene having from 1 to 10 carbon atoms and D is a quaternary ammonium group;
A₁ and B₁ represent polymethylene groups containing from 2 to 20 carbon atoms, which may be linear or branched and saturated or unsaturated and may contain, joined to or intercalated in the main chain, one or more aromatic rings, or one or more oxygen or sulphur atoms or sulphoxide, sulphone, disulphide, amino, alkylamino, hydroxyl, quaternary ammonium, ureido, amide or ester groups, and
X⁻ denotes an anion derived from an inorganic or organic acid;
A₁, R₁₃ and R₁₅ may, with the two nitrogen atoms to which they are attached, form a piperazine ring; moreover, if A₁ denotes a linear or branched, saturated or unsaturated alkylene or hydroxyalkylene group, B₁ may also denote a group

—(CH₂)ₙ—CO-D-OC—(CH₂)ₚ—

in which:
n and p are integers ranging from 2 to 20 approximately,
D denotes:
a) a glycol residue of formula: where Z denotes a linear or branched hydrocarbon-based group or a group corresponding to one of the following formulae:

—(CH₂—CH₂—O)ₓ—CH₂—CH₂—

—[CH₂—CH(CH₃)—O]_{y}—CH₂—CH(CH₃)—

where x and y denote an integer from 1 to 4, representing a defined and unique degree of polymerization or any number from 1 to 4 representing an average degree of polymerization;
b) a bis-secondary diamine residue such as a piperazine derivative;
c) a bis-primary diamine residue of formula: —NH—Y—NH—, where Y denotes a linear or branched hydrocarbon-based group, or else the divalent group —CH₂—CH₂—S—S—CH₂—CH₂—;
d) a ureylene group of formula: —NH—CO—NH—.

Preferably, X⁻ is an anion such as chloride or bromide.

These polymers generally have a number-average molecular weight of between 1000 and 100 000.

Polymers of this type are described in particular in French patents 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945,and 4,027,020.

Use may more particularly be made of the polymers which are composed of repeating units corresponding to the formula (XVIII):

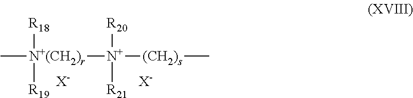

in which: R₁₈, R₁₀, R₂₀ and R₂₁, which are identical or different, denote an alkyl or hydroxyalkyl group having from 1 to 4 carbon atoms approximately, r and s are integers ranging from 2 to 20 approximately, and X⁻ is an anion derived from an inorganic or organic acid.

One particularly preferred compound of formula (XVIII) is that for which R₁₈, R₁₀, R₂₀ and R₂₁ represent a methyl group and r=3, s=6 and X=Cl, which is called Hexadimethrine chloride according to INCI nomenclature (CTFA).

(8) Polyquaternary ammonium polymers composed of units of formula (XIX):

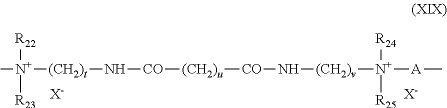

in which formula:
R₂₂, R₂₃, R₂₄ and R₂₅, which are identical or different, represent a hydrogen atom or a methyl, ethyl, propyl, β-hydroxyethyl, β-hydroxypropyl or —CH₂CH₂(OCH₂CH₂)ₚOH group, where p is equal to 0 or to an integer between 1 and 6, with the proviso that R₂₂, R₂₃, R₂₄ and R₂₅ do not simultaneously represent a hydrogen atom,
t and u, which are identical or different, are integers between 1 and 6,
v is equal to 0 or to an integer between 1 and 34,
X⁻ denotes an anion such as a halide,
A denotes a dihalide group or preferably represents —CH₂—CH₂—O—CH₂—CH₂—.

Such compounds are described especially in Patent Application EP-A-122 324.

Among these, mention may be made, for example, of the products Mirapol® A 15, Mirapol® AD1, Mirapol® AZ1 and Mirapol® 175, sold by the company Miranol.

(9) Quaternary polymers of vinylpyrrolidone and of vinylimidazole, for instance the products sold under the names Luviquat® FC 905, FC 550 and FC 370 by the company BASF.
(10) Cationic polysaccharides, in particular cationic celluloses and derivatives of cationic celluloses, and cationic galactomannan gums.

Among the cationic polysaccharides, mention may be made more particularly of cellulose ether derivatives comprising quaternary ammonium groups, cationic cellulose copolymers or cellulose derivatives grafted with a water-soluble quaternary ammonium monomer and cationic galactomannan gums.

The cellulose ether derivatives comprising quaternary ammonium groups described in French Patent 1 492 597. These polymers are also defined in the CTFA dictionary as quaternary ammoniums of hydroxyethylcellulose that have reacted with an epoxide substituted with a trimethylammonium group.

The cationic cellulose copolymers or the cellulose derivatives grafted with a water-soluble quaternary ammonium monomer are described especially in U.S. Pat. No. 4,131,576, such as hydroxyalkyl celluloses, for instance hydroxymethyl, hydroxyethyl or hydroxypropyl celluloses grafted especially with a methacryloylethyltrimethylammonium, methacrylamidopropyltrimethylammonium or dimethyldiallylammonium salt.

The cationic galactomannan gums are described more particularly in U.S. Pat. Nos. 3,589,578 and 4,031,307, in particular guar gums containing cationic trialkylammonium groups. Use is made, for example, of guar gums modified with a 2,3-epoxypropyltrimethylammonium salt (e.g. chloride).

Other cationic polymers that may be used in the context of the invention are cationic proteins or cationic protein hydrolysates, polyalkyleneimines, in particular polyethyleneimines, polymers containing vinylpyridine or vinylpyridinium units, condensates of polyamines and of epichlorohydrin, quaternary polyureylenes and chitin derivatives.

The cationic proteins or protein hydrolysates are, in particular, chemically modified polypeptides bearing quaternary ammonium groups at the end of the chain, or grafted thereon. Their molecular weight may vary, for example, from 1500 to 10 000 and in particular from 2000 to 5000 approximately. Among these compounds, mention may be made especially of:
collagen hydrolysates bearing triethylammonium groups, such as the products sold under the name Quat-Pro E by the company Maybrook and referred to in the CTFA dictionary as Triethonium Hydrolyzed Collagen Ethosulphate;
collagen hydrolysates bearing trimethylammonium chloride and trimethylstearylammonium chloride groups, which are sold under the name Quat-Pro S by the company Maybrook and are referred to in the CTFA dictionary as Steartrimonium Hydrolyzed Collagen;
animal protein hydrolysates bearing trimethylbenzylammonium groups, such as the products sold under the name Crotein BTA by the company Croda and referred to in the CTFA dictionary as Benzyltrimonium hydrolyzed animal protein;
protein hydrolysates bearing quaternary ammonium groups on the polypeptide chain, said ammonium groups containing at least one alkyl group having from 1 to 18 carbon atoms.

Among these protein hydrolysates, mention may be made, inter alia, of:
Croquat L, in which the quaternary ammonium groups contain a C₁₂ alkyl group;
Croquat M, in which the quaternary ammonium groups contain C₁₀-C₁₈ alkyl groups;
Croquat S, in which the quaternary ammonium groups contain a C₁₋₈ alkyl group;
Crotein Q, in which the quaternary ammonium groups contain at least one alkyl group having from 1 to 18 carbon atoms.

These various products are sold by the company Croda.

Other quaternized proteins or hydrolysates are, for example, those corresponding to the formula (XX):

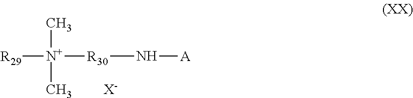

in which X⁻ is an anion of an organic or inorganic acid, A denotes a protein residue derived from collagen protein hydrolysates, R₂₉ denotes a lipophilic group containing up to 30 carbon atoms, and R₃₀ represents an alkylene group having 1 to 6 carbon atoms. Mention may, for example, be made of the products sold by the company Inolex, under the name Lexein QX 3000, called, in the CTFA dictionary, Cocotrimonium Collagen Hydrolysate.

Mention may also be made of quaternized plant proteins such as wheat, corn or soybean proteins: quaternized wheat proteins that may be mentioned include those sold by the company Croda under the names Hydrotriticum WQ or QM, which in the CTFA dictionary are called Cocodimonium Hydrolysed wheat protein, or Hydrotriticum QL, which in the CTFA dictionary is called Laurdimonium hydrolysed wheat protein, or else Hydrotriticum QS, which in the CTFA dictionary is called Steardimonium hydrolysed wheat protein.

Among all the cationic polymers that may be used in the context of the present invention, it is preferred to use cationic cyclopolymers, such as defined above, in particular the dimethyldiallylammonium chloride homopolymers or copolymers sold under the names Merquat 100, Merquat 550 and Merquat S by the company Nalco, and quaternary vinylpyrrolidone and vinylimidazole polymers, cationic polysaccharides and mixtures thereof.

The conditioning agent(s) that can be used according to the invention may be selected from cationic surfactants.

The term “cationic surfactant” means a surfactant that is positively charged when it is contained in the composition according to the invention. This surfactant may bear one or more positive permanent charges or may contain one or more cationizable functions within the composition according to the invention.

The cationic surfactant(s) that may be used as conditioning agents according to the present invention are preferably selected from optionally polyoxyalkylenated primary, secondary or tertiary fatty amines, or salts thereof, quaternary ammonium salts, and mixtures thereof.

The fatty amines generally comprise at least one C₈-C₃₀ hydrocarbon-based chain. Among the fatty amines that may be used according to the invention, examples that may be mentioned include stearylamidopropyldimethylamine and distearylamine.

Examples of quaternary ammonium salts that may especially be mentioned include:
those corresponding to the general formula (XXI) below:

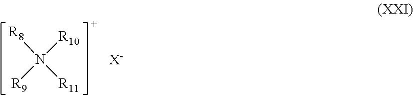

in which the groups R₈ to R₁₁, which may be identical or different, represent a linear or branched aliphatic group containing from 1 to 30 carbon atoms, or an aromatic group such as aryl or alkylaryl, at least one of the groups R₈ to R₁₁ denoting a group containing from 8 to 30 carbon atoms, preferably from 12 to 24 carbon atoms. The aliphatic groups can comprise heteroatoms such as, in particular, oxygen, nitrogen, sulphur and halogens. The aliphatic groups are selected, for example, from C₁₋₃₀ alkyl, C₁₋₃₀ alkoxy, polyoxyalkylene (C₂-C₆), C₁₋₃₀ alkylamide, (C₁₂-C₂₂)alkylamido(C₂-C₆)alkyl, (C₁₂-C₂₂)alkylacetate, and C₁₋₃₀ hydroxyalkyl groups; X⁻ is an anion selected from the group of halides, phosphates, acetates, lactates, (C₁-C₄)alkyl sulphates and (C₁-C₄)alkyl sulphonates or (C₁-C₄)alkylaryl sulphonates.

Among the quaternary ammonium salts of formula (XXI), those that are preferred are, on the one hand, tetraalkylammonium salts, for instance dialkyldimethyl-ammonium or alkyltrimethylammonium salts in which the alkyl group contains approximately from 12 to 22 carbon atoms, in particular behenyltrimethyl-ammonium, distearyldimethylammonium, cetyltrimethylammonium or benzyl-dimethylstearylammonium salts, or, on the other hand, the palmitylamidopropyl-trimethylammonium salt, the stearamidopropyltrimethylammonium salt, the stearamidopropyldimethylcetearylammonium salt, or the stearamidopropyl-dimethyl(myristyl acetate)ammonium salt sold under the name Ceraphyl® 70 by the company Van Dyk. It is particularly preferred to use the chloride salts of these compounds;
quaternary ammonium salts of imidazoline, such as, for example, those of formula (XXII) below:

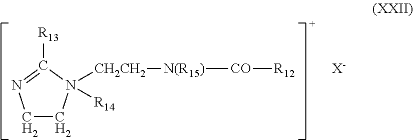

in which R₁₂ represents an alkenyl or alkyl group containing from 8 to 30 carbon atoms, for example tallow fatty acid derivatives, R₁₃ represents a hydrogen atom, a C₁-C₄ alkyl group or an alkenyl or alkyl group containing from 8 to 30 carbon atoms, R₁₄ represents a C₁-C₄ alkyl group, R₁₅ represents a hydrogen atom or a C₁-C₄ alkyl group, X⁻ is an anion selected from the group of halides, phosphates, acetates, lactates, alkyl sulphates, alkyl sulphonates or alkylaryl sulphonates, the alkyl and aryl groups of which preferably comprise, respectively, from 1 to 20 carbon atoms and from 6 to 30 carbon atoms. R₁₂ and R₁₃ preferably denote a mixture of alkyl or alkenyl groups comprising from 12 to 21 carbon atoms, for example tallow fatty acid derivatives, R₁₄ denotes a methyl group, and R₁₅ denotes a hydrogen atom. Such a product is sold, for example, under the name Rewoquat® W 75 by the company Rewo;
quaternary di- or triammonium salts of formula (XXIII):

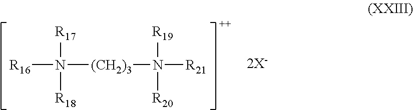

in which R₁₆ denotes an alkyl group containing approximately from 16 to 30 carbon atoms, which is optionally hydroxylated and/or interrupted with one or more oxygen atoms, R₁₇ is selected from hydrogen or an alkyl group containing from 1 to 4 carbon atoms or the following group:

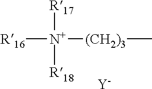

R′₁₆, R′₁₇, R′₁₈, R₁₈, R₁₉, R₂₀ and R₂₁, which may be identical or different, are selected from hydrogen or an alkyl group containing from 1 to 4 carbon atoms, and X⁻ and Y⁻ are anions in particular selected from the group of halides, acetates, phosphates, nitrates and (C₁-C₆)alkyl sulphates, in particular methyl sulphate or ethyl sulphate. Such compounds are, for example, Finquat CT-P from the company Finetex (Quaternium-89), Finquat CT from the company Finetex (Quaternium 75) and Condicate CT from the company Innospec Active Chemicals (Quaternium-75);
quaternary ammonium salts containing at least one ester function, such as those of formula (XXIV) below:

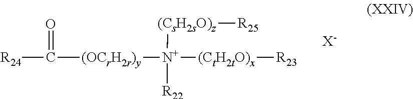

in which:
R₂₂ is selected from C₁-C₆ alkyl groups and C₁-C₆ hydroxyalkyl or dihydroxyalkyl groups;
R₂₃ is selected from:
the group

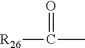

groups R₂₇ which are linear or branched, saturated or unsaturated C₁-C₂₂ hydrocarbon-based groups,
a hydrogen atom,
   R₂₅ is selected from:
the group

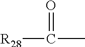

groups R₂₉ which are linear or branched, saturated or unsaturated C₁-C₆ hydrocarbon-based groups,
a hydrogen atom,
   R₂₄, R₂₆ and R₂₈, which may be identical or different, are selected from linear or branched, saturated or unsaturated C₇-C₂₁ hydrocarbon-based groups;
   r, s and t, which may be identical or different, are integers ranging from 2 to 6;
   y is an integer ranging from 1 to 10;
   x and z, which may be identical or different, are integers ranging from 0 to 10;
   X⁻ is a simple or complex, organic or inorganic anion; with the proviso that the sum x+y+z is from 1 to 15, that when x is 0, then R₂₃ denotes R₂₇ and that when z is 0, then R₂₅ denotes R₂₉.

The alkyl groups R₂₂ may be linear or branched, and more particularly linear.

Preferably, R₂₂ denotes a methyl, ethyl, hydroxyethyl or dihydroxypropyl group, and more particularly a methyl or ethyl group.

Advantageously, the sum x+y+z has a value from 1 to 10.

When R₂₃ is a hydrocarbon-based group R₂₇, it may be long and may contain from 12 to 22 carbon atoms, or may be short and may contain from 1 to 3 carbon atoms.

When R₂₅ is a hydrocarbon-based group R₂₉, it preferably contains 1 to 3 carbon atoms.

Advantageously, R₂₄, R₂₆ and R₂₈, which may be identical or different, are selected from linear or branched, saturated or unsaturated C₁₁-C₂₁ hydrocarbon-based groups, and more particularly from linear or branched, saturated or unsaturated C₁₁-C₂₁ alkyl and alkenyl groups.

Preferably, x and z, which are identical or different, have values of 0 or 1.

Advantageously, y is equal to 1.

Preferably, r, s and t, which may be identical or different, are equal to 2 or 3, and more particularly still are equal to 2.

The anion X⁻ is preferably a halide (chloride, bromide or iodide) or an alkyl sulphate, more particularly methyl sulphate. However, use may be made of methanesulphonate, phosphate, nitrate, tosylate, an anion derived from an organic acid, such as acetate or lactate, or any other anion compatible with the ammonium containing an ester function.

The anion X⁻ is even more particularly chloride or methyl sulphate.

Use is made more particularly, in the composition according to the invention, of the ammonium salts of formula (XXIV) in which:

R₂₂ denotes a methyl or ethyl group,
x and y are equal to 1;
z is equal to 0 or 1;
s and t are equal to 2;
R₂₃ is selected from:
the group

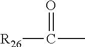

methyl, ethyl or C₁₄-C₂₂ hydrocarbon-based groups,
a hydrogen atom;
   R₂₅ is selected from:
the group

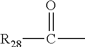

a hydrogen atom;
   R₂₄, R₂₆ and R₂₈, which may be identical or different, are selected from linear or branched, saturated or unsaturated C₁₃-C₁₇ hydrocarbon-based groups and preferably from linear or branched, saturated or unsaturated C₁₃-C₁₇ alkyl and alkenyl groups.

The hydrocarbon-based groups are advantageously linear.

Mention may be made, for example, of the compounds of formula (XXIV) such as the diacyloxyethyldimethylammonium, diacyloxyethylhydroxyethylmethyl-ammonium, monoacyloxyethyldihydroxyethylmethylammonium, triacyloxyethyl-methylammonium and monoacyloxyethylhydroxyethyldimethylammonium salts (chloride or methyl sulphate in particular), and mixtures thereof. The acyl groups preferably contain 14 to 18 carbon atoms and are obtained more particularly from a plant oil such as palm oil or sunflower oil. When the compound contains several acyl groups, these groups may be identical or different.

These products are obtained, for example, by direct esterification of triethanolamine, of triisopropanolamine, of alkyldiethanolamine or of alkyldiisopropanolamine, which are optionally alkoxylated, with C₁₀-C₃₀ fatty acids or with mixtures of C₁₀-C₃₀ fatty acids of plant or animal origin, or by transesterification of their methyl esters. This esterification is followed by a quaternization by means of an alkylating agent such as an alkyl (preferably methyl or ethyl)halide, a dialkyl (preferably methyl or ethyl)sulphate, methyl methanesulphonate, methyl para-toluenesulphonate, glycol chlorohydrin or glycerol chlorohydrin.

Such compounds are sold, for example, under the names Dehyquart® by the company Henkel, Stepanquat® by the company Stepan, Noxamium® by the company Ceca or Rewoquat® WE 18 by the company Rewo-Witco.

The composition according to the invention may contain, for example, a mixture of quaternary ammonium monoester, diester and triester salts with a majority by weight of diester salts.

Mixtures of ammonium salts that may be used include, for example, the mixture containing 15% to 30% by weight of acyloxyethyldihydroxyethylmethylammonium methyl sulphate, 45% to 60% of diacyloxyethylhydroxyethylmethylammonium methyl sulphate and 15% to 30% of triacyloxyethylmethylammonium methyl sulphate, the acyl groups having from 14 to 18 carbon atoms and originating from palm oil, which is optionally partially hydrogenated.

It is also possible to use the ammonium salts containing at least one ester function that are described in U.S. Pat. No. 4,874,454 and U.S. Pat. No. 4,137,180.

The particularly preferred cationic surfactant(s) that can be used according to the invention are selected from compounds of formula (XXI) or of formula (XXIV), methyl(C₉-C₁₉)alkyl(C₁₀-C₂₀)alkylamidoethylimidazolium salts, and stearamidopropyldimethylamine.

Among all of the cationic surfactants that may be present in the composition according to the invention, it is preferred to select cetyltrimethylammonium, behenyltrimethylammonium, di(palmitoyloxyethyl)hydroxyethylmethylammonium, di(stearoyloxyethyl)hydroxyethylmethylammonium, methyl(C₉-C₁₉)alkyl(C₁₀-C₂₀)-alkylamidoethylimidazolium salts, stearamidopropyltrimethylammonium salt, stearamidopropyldimethylamine, stearamidopropyldimethylcetearylammonium salt, and mixtures thereof.

The cationic surfactant(s) may be present in a content ranging from 0.01% to 15% by weight, preferably ranging from 0.1% to 10% by weight and more preferentially ranging from 0.2% to 5% by weight, with respect to the total weight of the composition of the invention.

When the composition in accordance with the invention comprises one or more surfactants, the product(s) (i)/cationic surfactant(s) weight ratio ranges from 0.1 to 50, more preferably still from 1 to 25, better still from 1 to 10.

Among the silicones that can be used as conditioning agents in accordance with the present invention, mention may be made, without limitation, of the following:

### I. Volatile Silicones:

These silicones have a boiling point of between 60° C. and 260° C. Among the silicones of this type that are mentioned are:

(a) cyclic silicones comprising from 3 to 7, and preferably 4 to 5, silicon atoms. These are, for example, the octamethylcyclotetrasiloxane sold under the name

Volatile Silicone 7207® by the company Union Carbide, or Silbione 70045 V2® by the company Rhône-Poulenc, the decamethylcyclopentasiloxane sold under the name Volatile Silicone 7158® by the company Union Carbide or Silbione 70045 V5® by the company Rhône-Poulenc, and mixtures thereof. Mention is also made of cyclopolymers of the dimethylsiloxane/methylalkylsiloxane type, such as Volatile Silicone FZ 3109® sold by the company Union Carbide, which is a dimethyl-siloxane/methyloctylsiloxane cyclocopolymer;

(b) linear volatile silicones containing 2 to 9 silicon atoms and having a viscosity of less than or equal to 5×10⁻⁶ m²/s at 25° C.

These are, for example, the hexamethyldisiloxane sold under the name “Silbione 70041V0.65® by the company Rhône-Poulenc. This type of product is described in the article by Todd & Byers “Volatile silicone fluids for cosmetics”, Cosmetics and Toiletries, Vol. 91, January 76, pages 27-32.

### II. Non-Volatile Silicones:

These silicones are mainly constituted by polyalkylsiloxanes, polyarylsiloxanes, polyalkylarylsiloxanes and organomodified polysiloxanes, and also mixtures thereof. They may be in the form of oils, gums and resins.

Among the polyalkylsiloxanes, mention may be made mainly of linear polydimethylsiloxanes with a viscosity of greater than 5×10⁻⁶ m²/s, and preferably less than 2.6 m²/s, i.e.:
containing trimethylsilyl end groups, such as, for example, and without limitation, the Silbione® oils of series 70047 that are sold by the company Rhône-Poulenc, the oil Wacker Belsil DM 60000 from Wacker, or certain Viscasil® products from the company General Electric;
containing trihydroxysilyl end groups of the 48 V® series from the company Rhône-Poulenc.

In this class of polyalkylsiloxanes, mention may also be made of the polyalkylsiloxanes sold by the company Goldschmidt under the names Abil Wax 9800® and Abil Wax 9801®, which are poly(C₁₋₂₀)alkylsiloxanes.

Among the polyalkylarylsiloxanes, mention may be made of linear and/or branched polydimethylphenylsiloxanes and polydimethyldiphenylsiloxanes, with a viscosity from 10⁻⁵ to 5×10⁻² m²/s, for instance:
the oil Rhodorsil® 763 from Rhône-Poulenc,
the Silbione® oils of the 70641 series from Rhône-Poulenc, such as the oils Silbione 70641 V30® and Silbione 70641 V200®,
the product DC 556® Cosmetic Grade Fluid from Dow Corning,
the silicones of the PK series from Bayer, such as PK20®,
the silicones of the PN and PH series from Bayer, such as the products PN1000® and PH1000®;
certain oils of the SF series from General Electric, such as SF 1250®, SF 1265®, SF 1154® and SF 1023®.

The silicone gums in accordance with the present invention are polydiorganosiloxanes with a high number-average molecular weight of between 200 000 and 1 000 000, used alone or as a mixture in a solvent selected from volatile silicones, polydimethylsiloxane (PDMS) oils, polyphenylmethylsiloxane (PPMS) oils, isoparaffins, methylene chloride, pentane, dodecane, tridecane and tetradecane, or mixtures thereof.

Mention is made, for example, of the compounds having the following structures:
poly[(dimethylsiloxane)/(methylvinylsiloxane)]gums,
poly[(dimethylsiloxane)/(diphenylsiloxane)]gums,
poly[(dihydrodimethylsiloxane)/(divinylsiloxane)]gums,
poly[(dimethylsiloxane)/(phenylmethylsiloxane)]gums,
poly[(dimethylsiloxane)/(diphenylsiloxane)/(methylvinylsiloxane)]gums.

The Mirasil DM 300 000 gum from the company Rhodia may be mentioned. Mention may also be made, for example, in a nonlimiting manner, of the following mixtures:

1) mixtures formed from a hydroxyl-terminated polydimethylsiloxane (dimethiconol according to CTFA nomenclature) and from a cyclic polydimethylsiloxane (cyclomethicone according to CTFA nomenclature), such as the products Q2 1401® or Dow Corning 1501 Fluid sold by the company Dow Corning;
2) mixtures formed from a polydimethylsiloxane gum with a cyclic silicone, such as the product SF 1214 Silicone Fluid® from General Electric, which is an SE 30® gum of MW 500 000 (−Mₙ) dissolved in SF 1202 Silicone Fluid® (decamethylcyclopentasiloxane);
3) mixtures of two PDMSs of different viscosities, especially of a PDMS gum and a PDMS oil, such as the products SF 1236® and CF 1241® from the company General Electric. The product SF 1236® is the mixture of an SE 30® gum defined above, with a viscosity of 20 m²/s, and of an SF 96® oil with a viscosity of 5×10⁶ m²/s (15% SE 30® gum and 85% SF 96® oil).

The product CF 1241® is the mixture of an SE 30® gum (33%) and of a PDMS (67%), with a viscosity of 10⁻³ m²/s.

The organopolysiloxane resins that may be used in accordance with the invention are crosslinked siloxane systems containing the following units: R₂SiO_{2/2}, RSiO_{3/2} and SiO_{4/2} in which R represents a hydrocarbon-based group containing 1 to 6 carbon atoms or a phenyl group. Among these products, those which are particularly preferred are those in which R denotes a lower (C₁-C₄) alkyl group or a phenyl group.

Among these resins, mention may be made of the product sold under the name Dow Corning 593® or those sold under the names Silicone Fluid SS 4230 and Silicone Fluid SS 4267 by the company General Electric, which are dimethyl/trimethylpolysiloxanes.

The organomodified silicones in accordance with the present invention are silicones as defined above, comprising, in their general structure, one or more organofunctional groups directly attached to the siloxane chain or attached via a hydrocarbon-based group.

Mention may be made, for example, of the silicones comprising:

a) perfluoro groups such as trifluoroalkyls, for instance those sold by the company General Electric under the names FF.150 Fluorosilicone Fluid® or by the company
Shin-Etsu under the names X-22-819®, X-22-82®, X-22-821® and X-22-822®;
b) hydroxyacylamino groups, for instance those described in Patent Application EP 0 342 834 and in particular the silicone sold by the company Dow Corning under the name Q2-8413®;
c) thiol groups, as in the silicones X 2-8360® from the company Dow Corning or GP 72A® and GP 71® from Genesee;
d) non-quaternized amine groups, such as GP 4 Silicone Fluid® from Genesee, GP 7100® from Genesee, Q2 8220® from Dow Corning, AFL 40® from Union Carbide or the silicone known as Amodimethicone in the CTFA dictionary;
e) carboxylate groups, for instance the products described in Patent EP 186 507 from Chisso Corporation;
f) hydroxyl groups, for instance the hydroxyalkyl-functional polyorganosiloxanes described in Patent Application FR 8 516 334, corresponding to the formula (XXV) below:

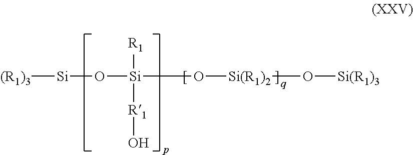

in which:
the R₁ groups, which are identical or different, are selected from methyl and phenyl groups, at least 60 mol % of the R₁ groups being methyl;
the R′₁ group is a divalent C₂-C₁₈ hydrocarbon-based alkylene chain member;
p is between 1 and 30 inclusive;
q is between 1 and 150 inclusive.

Mention may be made very particularly of the product sold by Dow Corning under the name DC 190;

g) alkoxylated groups, as in the silicone copolymer F 755® from SWS Silicones and the products Abil Wax 2428®, Abil Wax 2434® and Abil Wax 2440® from the company Goldschmidt;
h) acyloxyalkyl groups, such as, for example, the polyorganopolysiloxanes described in Patent Application FR 8 817 433, corresponding to the formula (XXVI) below:

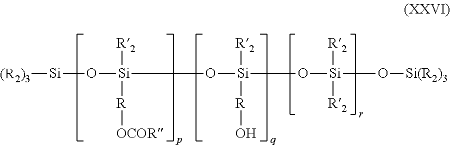

in which:
R₂ denotes methyl, phenyl, OCOR″ or hydroxyl, but only one R₂ per silicon atom may be OH;
R′₂ denotes methyl or phenyl, at least 60 mol % of the entirety of the R₂ and R′₂ groups being methyl;
R″ denotes C₈-C₂₀ alkyl or alkenyl;
R denotes a linear or branched, divalent C₂-C₁₈ hydrocarbon-based alkylene;
r is between 1 and 120 inclusive;
p, q is 0 or is less than 0.5 p, with p+q being between 1 and 30 inclusive: the polyorganosiloxanes of formula (XXVI) can comprise groups:

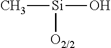

in proportions not exceeding 15% of the sum p+q+r;
i) quaternary ammonium groups, as in the products X2 8108® and X2 8109® and the product Abil K3270® from the company Goldschmidt;
j) amphoteric or betaine groups, such as in the product sold by the company Goldschmidt under the name Abil B 9950®;
k) bisulphite groups, such as in the products sold by the company Goldschmidt under the names Abil S 201® and Abil S 255®.
l) polyethyleneoxy and/or polypropyleneoxy groups optionally comprising C₆-C₂₄ alkyl groups, such as the products called dimethicone copolyol sold by the company Dow Corning under the name DC 1248, or the oils Silwet L 722, L 7500, L 77 and L 711 from the company Union Carbide, and the alkyl (C₁₂) methicone copolyol sold by the company Dow Corning under the name Q2 5200.

According to the invention, it is also possible to use silicones comprising a polysiloxane portion and a portion composed of a non-silicone organic chain, with one of the two portions making up the main chain of the polymer and the other being grafted onto said main chain. These polymers are described, for example, in Patent Applications EP-A-412 704, EP-A-412 707, EP-A-640 105 and WO 95/00578, EP-A-582 152 and WO 93/23009 and U.S. Pat. No. 4,693,935, U.S. Pat. No. 4,728,571 and U.S. Pat. No. 4,972,037. These polymers are preferably anionic or nonionic. Such polymers are, for example, copolymers that may be obtained by free radical polymerization from the monomer mixture formed from:

a) 50% to 90% by weight of tert-butyl acrylate;
b) 0 to 40% by weight of acrylic acid;
c) 5% to 40% by weight of silicone macromer of formula (XXVII):

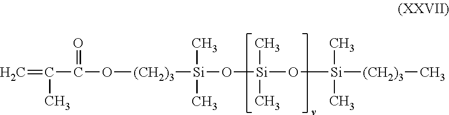

where v is a number ranging from 5 to 700; the percentages by weight are calculated relative to the total weight of the monomers.

Other examples of grafted silicone polymers are, in particular, polydimethylsiloxanes (PDMSs) onto which are grafted, by means of a connecting link of thiopropylene type, mixed polymer units of the poly((meth)acrylic acid) type and of the poly(alkyl (meth)acrylate) type; and polydimethylsiloxanes (PDMSs) onto which are grafted, by means of a connecting link of thiopropylene type, polymer units of the poly(isobutyl (meth)acrylate) type.

According to the invention, all the silicones can also be used in the form of emulsions, nanoemulsions or microemulsions.

The particularly preferred polyorganosiloxanes in accordance with the invention are as follows:
non-volatile silicones selected from the family of polyalkylsiloxanes with trimethylsilyl end groups, such as oils having a viscosity of between 0.2 and 2.5 m²/s at 25° C., for instance the oils of the DC200 series from Dow Corning, in particular the one with a viscosity of 60 000 cSt, or of the Silbione 70047 and 47 series, and more particularly the oil 70 047 V 500 000 sold by the company Rhodia Chimie, and polyalkylsiloxanes with dimethylsilanol end groups, such as dimethiconols, or polyalkylarylsiloxanes, for instance the oil Silbione 70641 V 200 sold by the company Rhodia Chimie;
polysiloxanes containing amino groups, such as amodimethicones or trimethylsilylamodimethicones.

The viscosities of the silicones may especially be determined by the standard ASTM D445-97 (viscometry).

When the conditioning agent of the composition according to the invention is a hydrocarbon, it is a linear or branched C₈-C₃₀ hydrocarbon.

Among the hydrocarbons which are liquid at ambient temperature and meet this definition, mention may be made in particular of isododecane, isohexadecane and its isomers (such as 2,2,4,4,6,6-heptamethylnonane), isoeicosane, isotetracosane, isomers of said compounds, n-nonadecane, n-dodecane, n-undecane, n-tridecane, n-pentadecane, and the mixtures of these hydrocarbons.

Use is preferably made according to the invention of isododecane or an isomer thereof.

When the conditioning agent is a fatty alcohol, this alcohol is a linear or branched, saturated or unsaturated C₈-C₃₀ alcohol. Among the latter, mention may, for example, be made of 2-butyloctanol, lauryl alcohol, 2-octyldodecanol, oleyl alcohol, isocetyl alcohol, isostearyl alcohol, behenyl alcohol and mixtures thereof.

When the conditioning agent is a fatty ester, this ester may be either an ester of a C₈-C₃₀ fatty acid and a C₁-C₃₀ alcohol, and especially an ester of a C₈-C₃₀ fatty acid and a C₈-C₃₀ fatty alcohol, or an ester of a C₁-C₇ acid or diacid and a C₈-C₃₀ fatty alcohol.

Among these esters, mention may, for example, be made of ethyl, isopropyl, 2-ethylhexyl and 2-octyldecyl palmitate, isopropyl, butyl, cetyl and 2-octyldecyl myristate, butyl and hexyl stearate, hexyl and 2-hexyldecyl laurate, isononyl isononanoate, dioctyl malate, myristyl myristate, cetyl palmitate, and mixtures thereof.

The ceramides or ceramide analogues, such as glycoceramides, that can be used as conditioning agent in the compositions according to the invention are known per se and are natural or synthetic molecules that may correspond to the general formula (XXVIII) below:

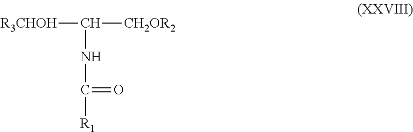

in which:
R₁ denotes a linear or branched, saturated or unsaturated alkyl group, derived from C₁₄-C₃₀ fatty acids, it being possible for this group to be substituted with a hydroxyl group in the alpha position, or a hydroxyl group in the omega position esterified with a saturated or unsaturated C₁₆-C₃₀ fatty acid;
R₂ denotes a hydrogen atom or a (glycosyl)ₙ, (galactosyl)ₘ or sulphogalactosyl group, in which n is an integer ranging from 1 to 4 and m is an integer ranging from 1 to 8;
R₃ denotes a C₁₅-C₂₆ hydrocarbon-based group which is saturated or unsaturated in the alpha position, it being possible for this group to be substituted with one or more C₁-C₁₄ alkyl groups;
it being understood that, in the case of natural ceramides or glycoceramides, R₃ can also denote a C₁₅-C₂₆ alpha-hydroxyalkyl group, the hydroxyl group being optionally esterified with a C₁₆-C₃₀ alpha-hydroxy acid.

The ceramides which are preferred in the context of the present invention are those described by Downing in Arch. Dermatol., Vol. 123, 1381-1384, 1987, or those described in French patent FR 2 673 179.

The ceramide(s) that are more particularly preferred according to the invention are the compounds for which R₁ denotes a saturated or unsaturated alkyl derived from C₁₆-C₂₂ fatty acids; R₂ denotes a hydrogen atom; and R₃ denotes a saturated linear C₁₅ group.

Such compounds are, for example:
N-linoleoyl dihydrosphingosine,
N-oleyldihydrosphingosine,
N-palmitoyldihydrosphingosine,
N-stearoyl dihydrosphingosine,
N-behenoyl dihydrosphingosine,
   or mixtures of these compounds.

More preferably still, ceramides are used for which R₁ denotes a saturated or unsaturated alkyl group derived from fatty acids; R₂ denotes a galactosyl or sulphogalactosyl group; and R₃ denotes a —CH═CH—(CH₂)₁₂—CH₃ group.

Among all of these conditioning agents, preference is given to the use of one or more conditioning agents selected from silicones such as organosiloxanes and cationic polymers.

The cosmetic composition according to the invention preferably contains from 0.01% to 20% by weight, and more preferably from 0.05% to 10% by weight of conditioning agent(s), relative to the total weight of the composition.

The composition according to the invention may additionally comprise one or more conventional additives well known in the art, other than the compounds defined above. Mention may be made, as examples of additives which can be used according to the invention, of silanes, anionic surfactants, amphoteric or zwitterionic surfactants, nonionic surfactants, proteins, protein hydrolysates, vitamins, reducing agents, plasticizers, softeners, antifoaming agents, moisturizing agents, pigments, clays, inorganic fillers, UV screening agents, abrasive agents (pumice, apricot kernel powder), inorganic colloids, peptizing agents, solubilizing agents, fragrances, preservatives, pearlescent agents, propellants, antidandruff agents (for example, zinc pyrithione, octopirox, selenium sulphide, ellagic acid and derivatives), agents for combating hair loss or agents for promoting hair regrowth; these additives being other than the compounds defined above.

A person skilled in the art will take care to select the optional additive(s) and the amount thereof such that they do not harm the properties of the compositions of the present invention.

The additive(s) are generally present in the composition according to the invention in an amount ranging from 0 to 20% by weight relative to the total weight of the composition.

The compositions according to the invention may be provided in all the formulation forms conventionally used for a topical application and in particular in the form of aqueous or aqueous/alcoholic solutions, of oil-in-water (O/W), water-in-oil (W/O) or multiple (triple: W/O/W or O/W/O) emulsions, of aqueous gels or of dispersions of a fatty phase in an aqueous phase using spherules, it being possible for these spherules to be polymeric nanoparticles, such as nanospheres and nanocapsules, or lipid vesicles of ionic and/or nonionic type (liposomes, niosomes or oleosomes).

These compositions are prepared according to the usual methods.

In addition, the compositions used according to the invention can be more or less fluid and can have the appearance of a white or coloured cream, of an ointment, of a milk, of a lotion, of a serum, of a paste or of a foam. They can optionally be applied to the keratin materials in aerosol form. They may also be in solid form, for example in stick form.

Of course, the person skilled in the art will take care to choose the optional compound or compounds to be added to the composition according to the invention so that the advantageous properties intrinsically attached to the composition in accordance with the invention are not, or not substantially, adversely affected by the envisaged addition or additions.

As indicated above, a further subject of the invention is a method for the nontherapeutic cosmetic treatment of keratin materials, in particular the hair, comprising the application, to said materials, of a composition as described above. This application may or may not be followed by a rinsing operation.

When the application of the composition is followed by a rinsing operation, the leave-in time of the composition on the keratin materials ranges from a few seconds to 60 minutes, better still from 5 seconds to 30 minutes, even better still from 10 seconds to 10 minutes.

Whether in rinsed mode or non-rinsed mode, the application of the composition may take place in the presence of heat. The heating device may be a hairdryer, a hood dryer, a curling iron or a flat iron. The heating temperature may be between 40° C. and 220° C.

The application of the composition according to the invention to the hair may take place on dry hair or on wet hair. It may in particular be carried out after a shampooing operation or after a pretreatment at acidic or basic pH.

Another subject of the invention is the use of a composition according to the invention for caring for keratin materials, especially keratin fibres and in particular human keratin fibres such as the hair.

The examples which follow are intended to illustrate the invention without however, exhibiting a limiting nature.

### EXAMPLES

Compositions according to the invention for provision of smoothness, softness, ease of combing and sheen:

| Composition | A |
|---|---|
| Product (A) of the reaction a) of an oxirane or oxetane | 10 g AM |
| compound comprising at least two oxirane or oxetane groups | |
| and b) of an aminosilane corresponding to the formula (Ia) | |
| (polymer A from WO 2009/061360) | |
| Acrylic thickening polymer (ii) | 0.2 g AM |
| pH agent | qs pH = 7 |
| Lactic acid | 0.5 g AM |
| Water | qs 100 g |

AM = active material

The acrylic thickening polymer (ii) is one of the following polymers:
30% acrylic polymer emulsion sold under the name Carbopol Aqua SF-1 by the company Lubrizol (example 1);
crosslinked carboxyvinyl homopolymer synthesized in the ethyl acetate/cyclohexane mixture sold under the name Carbopol Ultrez 10 by the company Lubrizol (example 2);
carboxyvinyl polymer synthesized in methylene chloride sold under the name Synthalen M by the company 3V (example 3);
acrylic acid/stearyl methacrylate copolymer polymerized in an ethyl acetate/cyclohexane mixture sold under the name Pemulen TR-1 by the company Lubrizol (example 4);
crosslinked acrylic acid/alkyl acrylate polymer sold under the name Pemulen TR-2 by the company Lubrizol (example 5).

1. Cosmetic composition comprising:
(i) one or more products (A) of the reaction of:
(a) one or more oxirane or oxetane compounds comprising at least two oxirane or oxetane groups; and
(b) one or more aminosilanes corresponding to the formula (Ia):
N(H)(R¹)R²Si(OR³)_{3-a-b-c}(OR⁴)ₐ(R⁵Si(OR⁶)_{d}(R⁷)ₑ)_{b}R⁸_{c} (Ia)
in which:
R¹ is selected from the group constituted by H or a monovalent hydrocarbon-based radical comprising from 1 to 20 carbon atoms;
R² is selected from the group constituted by a divalent, linear or branched, hydrocarbon-based radical, comprising from 1 to 60 carbons;
R⁴ is a hydrocarbon-based radical containing 3 to 200 carbon atoms;
R⁵ is selected from the group constituted by an oxygen atom or a divalent, linear or branched, hydrocarbon-based radical, comprising from 1 to 60 carbons;
R³, R⁶, R⁷ and R⁸ are each independently selected from the group constituted by monovalent, linear or branched, hydrocarbon-based radicals having from 1 to 200 carbon atoms;
the subscript b is equal to zero or is a positive number and has a value ranging from 0 to 3;
the subscript a is equal to zero or is a positive number below 3, the subscripts b and c are equal to zero or are positive and have a value ranging from 0 to 3 on condition that (a+b+c)≦3;
the subscripts d and e are equal to zero or are positive and have a value ranging from 0 to 3 on condition that (d+e)=3; and
(ii) one or more acrylic thickening polymers.

2. Composition according to claim 1, in which the oxirane or oxetane compound(s) is (are) selected from the group constituted by siloxanes, hydrocarbons and polyethers.

3. Composition according to either one of claims 1 and 2, in which the oxirane or oxetane compound(s) is (are):
either a siloxane corresponding to formula (Ib):
M_{f}M^{E}ₕM^{PE}ᵢM^{H}ⱼDₖD^{E}ₗD^{PE}ₘD^{H}ₙTₒT^{PE}_{q}T^{H}ᵣQₛ with
M=R⁹R¹⁰R¹¹SiO_{1/2};
M^{H}=R¹²R¹³HSiO_{1/2};
M^{PE}=R¹²R¹³(—CH₂CH(R¹⁴)(R¹⁵)ₜO(R¹⁶)ᵤ(C₂H₄O)ᵥ(C₃H₆O)_{w}(C₄H₈O)ₓR¹⁷)SiO_{1/2};
M^{E}=R¹²R¹³(R^{E})SiO_{1/2};
D=R¹⁸R¹⁹SiO_{2/2}; and
D^{H}=R²⁰HSiO_{2/2};
D^{PE}=R²⁰(—CH₂CH(R¹⁴)(R¹⁵)ₜO(R¹⁶)ᵤ(C₂H₄O)ᵥ(C₃H₆O)_{w}(C₄H₈O)ₓR¹⁷)SiO_{2/2},
D^{E}=R²⁰R^{E}SiO_{2/2};
T=R²¹SiO_{3/2};
T^{H}=HSiO_{3/2};
T^{PE}=(—CH₂CH(R¹⁴)(R¹⁵)ₜO(R¹⁶)ᵤ(C₂H₄O)ᵥ(C₃H₆O)_{w}(C₄H₈O)ₓR¹⁷)SiO_{3/2};
T^{E}=R^{E}SiO_{3/2}; and
Q=SiO_{4/2};
where R⁹, R¹⁰, R¹¹, R¹², R¹³, R¹⁸, R¹⁹, R²⁰ and R²¹ are each independently selected from the group constituted by monovalent, hydrocarbon-based radicals having from 1 to 60 carbon atoms;
R¹⁴ is H or an alkyl group having 1 to 6 carbon atoms; R¹⁵ is a divalent alkyl radical having 1 to 6 carbons; R¹⁶ is selected from the group constituted by divalent radicals constituted by —C₂H₄O—, —C₃H₆O—, and —C₄H₈O—; R¹⁷ is H, a monofunctional hydrocarbon-based radical having 1 to 6 carbons, or acetyl;
R^{E} is independently a monovalent hydrocarbon-based radical containing one or more oxirane or oxetane units having from one to sixty carbon atoms;
the subscript f may be equal to zero or be positive on condition that when the subscript f is equal to zero, h must be positive;
the subscript h may be equal to zero or be positive on condition that when h is equal to zero, the subscript f must be positive; and that the sum of the subscripts h, l and p is positive;
the subscript k is equal to zero or is positive and has a value ranging from around 0 to around 1000;
the subscript l is equal to zero or is positive and has a value ranging from around 0 to around 400 on condition that the sum of the subscripts h, l and p is positive;
the subscript o is equal to zero or is positive and has a value ranging from 0 to around 50;
the subscript p is equal to zero or is positive and has a value ranging from 0 to around 30 on condition that the sum of the subscripts h, l and p is positive;
the subscript s is equal to zero or is positive and has a value ranging from 0 to around 20;
the subscript i is equal to zero or is positive and has a value ranging from 0 to around 20;
the subscript m is equal to zero or is positive and has a value ranging from 0 to around 200;
the subscript q is equal to zero or is positive and has a value ranging from 0 to around 30;
the subscript j is equal to zero or is positive and has a value ranging from 0 to around 2;
the subscript n is equal to zero or is positive and has a value ranging from 0 to around 20;
the subscript r is equal to zero or is positive and has a value ranging from 0 to around 30;
the subscript t is equal to zero or one;
the subscript u is equal to zero or one;
the subscript v is equal to zero or is positive and has a value ranging from 0 to around 100 on condition that (v+w+x)>0;
the subscript w is equal to zero or is positive and has a value ranging from 0 to around 100 on condition that (v+w+x)>0;
the subscript x is equal to zero or is positive and has a value ranging from 0 to around 100 on condition that (v+w+x)>0;
or a hydrocarbon corresponding to formula (Ic):
R²²_{y}(R²³)_{z}(R²⁴_{α})(R²⁵)_{β} (Ic)
where R²² and R²⁵ are independently a monovalent hydrocarbon-based radical containing one or more oxirane or oxetane units having from 3 to 12 carbon atoms;
R²³ and R²⁴ are each selected from the group constituted by H or a monovalent, linear or branched, hydrocarbon-based radical having 1 to 200 carbons;
the subscripts y, z, α and β are equal to zero or are positive ranging from zero to four, on condition that (y+β)>2;
or a polyether corresponding to formula (Id):
R²⁶O(R²⁷)_{γ}(C₂H₄O)_{δ}(C₃H₆O)_{∈}(C₄H₈O)_{ξ}R²⁸ (Id)
where R²⁶ and R²⁸ are independently a monovalent hydrocarbon-based radical containing one or more oxirane or oxetane units having from 3 to 12 carbon atoms;
R²⁷ is selected from the group constituted by divalent radicals constituted by —C₂H₄O—, —C₃H₆O—, and —C₄H₈O—;
the subscript γ is equal to zero or 1;
the subscript δ is equal to zero or is positive and has a value ranging from 0 to around 100 on condition that (δ+∈+ξ)>0;
the subscript E is equal to zero or is positive and has a value ranging from 0 to around 100 on condition that (δ+∈+ξ)>0;
the subscript is equal to zero or is positive and has a value ranging from 0 to around 100 on condition that (δ+∈+ξ)>0.

4. Composition according to any one of claims 1 to 3, in which the synthesis of the product (A) may, besides an oxirane or oxetane compound and an aminosilane, also use a compound corresponding to formula (Ie):
R²⁹(R³⁰)_{κ}Si(OR³¹)_{3-η-θ}(R³²)_{η}(OR³³)_{θ} (Ie)
where R²⁹ is a monovalent hydrocarbon-based radical containing one or more oxirane or oxetane units having from 3 to 12 carbon atoms;
R³⁰ is a divalent hydrocarbon-based radical constituted of 1-60 carbons and the subscript κ has a value of zero or 1; R³¹ and R³² are independently selected from the group constituted by monovalent, linear or branched, hydrocarbon-based radicals having from 1 to 60 carbon atoms;
the subscript η is equal to zero or is positive and has a value ranging from 0 to 3;
the subscript θ is greater than 0 and less than or equal to 3, on condition that 3-η-θ is greater than or equal to zero;
R³³ is a hydrocarbon-based radical containing from 3 to 200 carbon atoms.

5. Composition according to any one of claims 1 to 4, in which the reaction of component a) with component b) may be carried out in the presence of a primary or secondary amine which may or may not have a reactive alkoxysilane unit.

6. Composition according to any one of claims 1 to 5, comprising from 0.01% to 20%, in particular from 0.05% to 15%, preferably from 0.5% to 15% by weight of one or more products (A) relative to the total weight of the composition.

7. Composition according to any one of claims 1 to 6, in which the acrylic thickening polymer(s) is (are) selected from:
(a) acrylic associative thickeners;
(b) crosslinked acrylic acid homopolymers;
(c) crosslinked copolymers of (meth)acrylic acid and of (C₁-C₆)alkyl acrylate;
(d) nonionic homopolymers and copolymers containing ethylenically unsaturated monomers of ester and/or amide type;
(e) ammonium acrylate homopolymers or copolymers of ammonium acrylate and of acrylamide;
(f) (meth)acrylamido(C₁-C₄)alkylsulphonic acid homopolymers and copolymers;
(g) crosslinked methacryloyl(C₁-C₄)alkyltri(C₁-C₄)alkylammonium homopolymers and copolymers.

8. Composition according to any one of claims 1 to 7, in which the acrylic thickening polymer(s) is (are) selected from:
(a) acrylic associative thickeners;
(b) crosslinked acrylic acid homopolymers;
(c) crosslinked copolymers of (meth)acrylic acid and of a (C₁-C₆)alkyl acrylate.

9. Composition according to claim 8, in which the acrylic associative thickening polymer(s) is (are) selected from acrylic anionic amphiphilic polymers comprising at least one hydrophilic unit of formula (III), and at least one hydrophobic unit of (C₁₀-C₃₀)alkyl ester of an unsaturated carboxylic acid type of formula (IV) below: 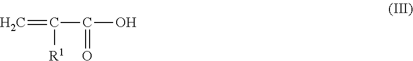
in which formula R¹ denotes H or CH₃ or C₂H₅, i.e. acrylic acid, methacrylic acid or ethacrylic acid units, and the hydrophobic unit of which, of (C₁₀-C₃₀)alkyl ester of an unsaturated carboxylic acid type, corresponds to the monomer of formula (IV) below: 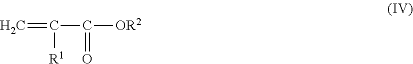
in which formula R¹ denotes H or CH₃ or C₂H₅ (i.e. acrylate, methacrylate or ethacrylate units) and preferably H (acrylate units) or CH₃ (methacrylate units), R² denoting a C₁₀-C₃₀ and preferably C₁₂-C₂₂ alkyl radical.

10. Composition according to any one of claims 1 to 9, in which the acrylic thickening polymer(s) represent(s) from 0.001% to 20% by weight, preferably from 0.01% to 10% by weight and more particularly from 0.1% to 5% by weight relative to the total weight of the composition.

11. Method for the nontherapeutic cosmetic treatment of keratin materials, especially keratin fibres and in particular human keratin fibres such as the hair, comprising the application, to said materials, of a composition as described in any one of claims 1 to 10.

12. Method according to claim 11, such that the composition is not rinsed out after application.

13. Method according to claim 11, such that the composition is rinsed out after application.

14. Method according to claims 11 to 13, such that the application of the composition takes place in the presence of heating between 40° C. and 220° C.

15. Use of a composition as defined according to one of claims 1 to 10, for caring for keratin materials, especially keratin fibres and in particular human keratin fibres such as the hair.
